SEQUENCE LISTING (180 sequences)

SEQ ID NO: 1 - protein, 5 aa, synthetic construct
  REGION 1..5: note = Description of Artificial Sequence: Syntheticpeptide
SYAMH

SEQ ID NO: 2 - protein, 5 aa, synthetic construct
  REGION 1..5: note = Description of Artificial Sequence: Syntheticpeptide
QYAMH

SEQ ID NO: 3 - protein, 5 aa, synthetic construct
  REGION 1..5: note = Description of Artificial Sequence: Syntheticpeptide
SYSMH

SEQ ID NO: 4 - protein, 5 aa, synthetic construct
  REGION 1..5: note = Description of Artificial Sequence: Syntheticpeptide
SYAIS

SEQ ID NO: 5 - protein, 17 aa, synthetic construct
  REGION 1..17: note = Description of Artificial Sequence: Syntheticpeptide
WINAGNGNTKYSQKFQG

SEQ ID NO: 6 - protein, 17 aa, synthetic construct
  REGION 1..17: note = Description of Artificial Sequence: Syntheticpeptide
WINAVSGDTKYSQKFQG

SEQ ID NO: 7 - protein, 17 aa, synthetic construct
  REGION 1..17: note = Description of Artificial Sequence: Syntheticpeptide
WINAGTGDTKYSQKFQG

SEQ ID NO: 8 - protein, 17 aa, synthetic construct
  REGION 1..17: note = Description of Artificial Sequence: Syntheticpeptide
WINEGYGNTKYSQKFQG

SEQ ID NO: 9 - protein, 17 aa, synthetic construct
  REGION 1..17: note = Description of Artificial Sequence: Syntheticpeptide
WINAGYGYTKYSQKFQG

SEQ ID NO: 10 - protein, 17 aa, synthetic construct
  REGION 1..17: note = Description of Artificial Sequence: Syntheticpeptide
WINAGTGNTKYSQKFQG

SEQ ID NO: 11 - protein, 17 aa, synthetic construct
  REGION 1..17: note = Description of Artificial Sequence: Syntheticpeptide
WINAGYGNTKYSQKFQG

SEQ ID NO: 12 - protein, 17 aa, synthetic construct
  REGION 1..17: note = Description of Artificial Sequence: Syntheticpeptide
WINAWNGNTKYSQKFQG

SEQ ID NO: 13 - protein, 17 aa, synthetic construct
  REGION 1..17: note = Description of Artificial Sequence: Syntheticpeptide
WINVGTGTTKYSQKFQG

SEQ ID NO: 14 - protein, 17 aa, synthetic construct
  REGION 1..17: note = Description of Artificial Sequence: Syntheticpeptide
WINAVNGNTKYSQKFQG

SEQ ID NO: 15 - protein, 17 aa, synthetic construct
  REGION 1..17: note = Description of Artificial Sequence: Syntheticpeptide
WINAGNWNTKYSQKFQG

SEQ ID NO: 16 - protein, 17 aa, synthetic construct
  REGION 1..17: note = Description of Artificial Sequence: Syntheticpeptide
WINAWTGNTKYSQKFQG

SEQ ID NO: 17 - protein, 17 aa, synthetic construct
  REGION 1..17: note = Description of Artificial Sequence: Syntheticpeptide
GIIPIFGTANYAQKFQG

SEQ ID NO: 18 - protein, 10 aa, synthetic construct
  REGION 1..10: note = Description of Artificial Sequence: Syntheticpeptide
NWGMSYGMDV

SEQ ID NO: 19 - protein, 9 aa, synthetic construct
  REGION 1..9: note = Description of Artificial Sequence: Syntheticpeptide
GYDSRPLDV

SEQ ID NO: 20 - protein, 9 aa, synthetic construct
  REGION 1..9: note = Description of Artificial Sequence: Syntheticpeptide
GYDSRPLDY

SEQ ID NO: 21 - protein, 11 aa, synthetic construct
  REGION 1..11: note = Description of Artificial Sequence: Syntheticpeptide
RASQSISSYLN

SEQ ID NO: 22 - protein, 11 aa, synthetic construct
  REGION 1..11: note = Description of Artificial Sequence: Syntheticpeptide
RASQSISPYLN

SEQ ID NO: 23 - protein, 11 aa, synthetic construct
  REGION 1..11: note = Description of Artificial Sequence: Syntheticpeptide
RASQSILSYLN

SEQ ID NO: 24 - protein, 11 aa, synthetic construct
  REGION 1..11: note = Description of Artificial Sequence: Syntheticpeptide
RASQSISWYLN

SEQ ID NO: 25 - protein, 11 aa, synthetic construct
  REGION 1..11: note = Description of Artificial Sequence: Syntheticpeptide
RASQSITSYLN

SEQ ID NO: 26 - protein, 11 aa, synthetic construct
  REGION 1..11: note = Description of Artificial Sequence: Syntheticpeptide
GGNNIGSKIVH

SEQ ID NO: 27 - skipped

SEQ ID NO: 28 - protein, 7 aa, synthetic construct
  REGION 1..7: note = Description of Artificial Sequence: Syntheticpeptide
AASSLQS

SEQ ID NO: 29 - protein, 7 aa, synthetic construct
  REGION 1..7: note = Description of Artificial Sequence: Syntheticpeptide
SASSLQS

SEQ ID NO: 30 - protein, 7 aa, synthetic construct
  REGION 1..7: note = Description of Artificial Sequence: Syntheticpeptide
SESSLQS

SEQ ID NO: 31 - protein, 7 aa, synthetic construct
  REGION 1..7: note = Description of Artificial Sequence: Syntheticpeptide
SSSSLQS

SEQ ID NO: 32 - protein, 7 aa, synthetic construct
  REGION 1..7: note = Description of Artificial Sequence: Syntheticpeptide
DDRDRPS

SEQ ID NO: 33 - protein, 10 aa, synthetic construct
  REGION 1..10: note = Description of Artificial Sequence: Syntheticpeptide
QQSYSTPALT

SEQ ID NO: 34 - protein, 10 aa, synthetic construct
  REGION 1..10: note = Description of Artificial Sequence: Syntheticpeptide
QQAYSTPALS

SEQ ID NO: 35 - protein, 11 aa, synthetic construct
  REGION 1..11: note = Description of Artificial Sequence: Syntheticpeptide
QVWDINVHHVI

SEQ ID NO: 36 - protein, 119 aa, synthetic construct
  REGION 1..119: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSTSTAYMELRSLRSDDTAMYYCARNWGMSYGMDVWGQGTMVTVSS

SEQ ID NO: 37 - protein, 119 aa, synthetic construct
  REGION 1..119: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARNWGMSYGMDVWGQGTTVTVSS

SEQ ID NO: 38 - protein, 119 aa, synthetic construct
  REGION 1..119: note = Description of Artificial Sequence: Syntheticpolypeptide
  VAR_SEQ 1: note = Gln or pyroglutamate
XVQLVQSGAEVKKPGASVKVSCKASGYTFTQYAMHWVRQAPGQRLEWMGWINAVSGDTKYSQKFQGRVTITRDTSTSTAYMELRSLRSDDTAMYYCARNWGMSYGMDVWGQGTMVTVSS

SEQ ID NO: 39 - protein, 119 aa, synthetic construct
  REGION 1..119: note = Description of Artificial Sequence: Syntheticpolypeptide
  VAR_SEQ 1: note = Gln or pyroglutamate
XVQLVQSGAEVKKPGASVKVSCKASGYTFTQYAMHWVRQAPGQRLEWMGWINAVSGDTKYSQKFQGRVTITRDTSASTAYMELSSLRSEDTAVYYCARNWGMSYGMDVWGQGTTVTVSS

SEQ ID NO: 40 - protein, 119 aa, synthetic construct
  REGION 1..119: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGTGDTKYSQKFQGRVTITRDTSTSTAYMELRSLRSDDTAMYYCARNWGMSYGMDVWGQGTMVTVSS

SEQ ID NO: 41 - protein, 119 aa, synthetic construct
  REGION 1..119: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGTGDTKYSQKFQGRVTITRDTSASTAYMELSSLRSEDTAVYYCARNWGMSYGMDVWGQGTTVTVSS

SEQ ID NO: 42 - protein, 119 aa, synthetic construct
  REGION 1..119: note = Description of Artificial Sequence: Syntheticpolypeptide
  VAR_SEQ 1: note = Gln or pyroglutamate
XVQLVQSGAEVKKPGASVKVSCKASGYTFNQYAMHWVRQAPGQRLEWMGWINEGYGNTKYSQKFQGRVTITRDTSTSTAYMELRSLRSDDTAMYYCARNWGMSYGMDVWGQGTMVTVSS

SEQ ID NO: 43 - protein, 119 aa, synthetic construct
  REGION 1..119: note = Description of Artificial Sequence: Syntheticpolypeptide
  VAR_SEQ 1: note = Gln or pyroglutamate
XVQLVQSGAEVKKPGASVKVSCKASGYTFTQYAMHWVRQAPGQRLEWMGWINAGYGYTKYSQKFQGRVTITRDTSTSTAYMELRSLRSDDTAMYYCARNWGMSYGMDVWGQGTMVTVSS

SEQ ID NO: 44 - protein, 119 aa, synthetic construct
  REGION 1..119: note = Description of Artificial Sequence: Syntheticpolypeptide
  VAR_SEQ 1: note = Gln or pyroglutamate
XVQLVQSGAEVKKPGASVKVSCKASGYTFTQYAMHWVRQAPGQRLEWMGWINAGYGYTKYSQKFQGRVTITRDTSASTAYMELSSLRSEDTAVYYCARNWGMSYGMDVWGQGTTVTVSS

SEQ ID NO: 45 - protein, 119 aa, synthetic construct
  REGION 1..119: note = Description of Artificial Sequence: Syntheticpolypeptide
  VAR_SEQ 1: note = Gln or pyroglutamate
XVQLVQSGAEVKKPGASVKVSCKASGYTFSQYAMHWVRQAPGQRLEWMGWINAGTGNTKYSQKFQGRVTITRDTSTSTAYMELRSLRSDDTAMYYCARNWGMSYGMDVWGQGTMVTVSS

SEQ ID NO: 46 - protein, 119 aa, synthetic construct
  REGION 1..119: note = Description of Artificial Sequence: Syntheticpolypeptide
  VAR_SEQ 1: note = Gln or pyroglutamate
XVQLVQSGAEVKKPGASVKVSCKASGYTFSQYAMHWVRQAPGQRLEWMGWINAGTGNTKYSQKFQGRVTITRDTSASTAYMELSSLRSEDTAVYYCARNWGMSYGMDVWGQGTTVTVSS

SEQ ID NO: 47 - protein, 119 aa, synthetic construct
  REGION 1..119: note = Description of Artificial Sequence: Syntheticpolypeptide
  VAR_SEQ 1: note = Gln or pyroglutamate
XVQLVQSGAEVKKPGASVKVSCKASGYTFDQYAMHWVRQAPGQRLEWMGWINAGYGNTKYSQKFQGRVTITRDTSTSTAYMELRSLRSDDTAMYYCARNWGMSYGMDVWGQGTMVTVSS

SEQ ID NO: 48 - protein, 119 aa, synthetic construct
  REGION 1..119: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSTSTAYMELRSLRSDDTAVYYCARNWGMSYGMDVWGQGTTVTVSS

SEQ ID NO: 49 - protein, 119 aa, synthetic construct
  REGION 1..119: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAWNGNTKYSQKFQGRVTITRDTSASTAYMELSSLRSEDTAVYYCARNWGMSYGMDVWGQGTTVTVSS

SEQ ID NO: 50 - protein, 119 aa, synthetic construct
  REGION 1..119: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINVGTGTTKYSQKFQGRVTITRDTSASTAYMELSSLRSEDTAVYYCARNWGMSYGMDVWGQGTTVTVSS

SEQ ID NO: 51 - protein, 119 aa, synthetic construct
  REGION 1..119: note = Description of Artificial Sequence: Syntheticpolypeptide
  VAR_SEQ 1: note = Gln or pyroglutamate
XVQLVQSGAEVKKPGASVKVSCKASGYTFSSYAMHWVRQAPGQRLEWMGWINAVNGNTKYSQKFQGRVTITRDTSASTAYMELSSLRSEDTAVYYCARNWGMSYGMDVWGQGTTVTVSS

SEQ ID NO: 52 - protein, 119 aa, synthetic construct
  REGION 1..119: note = Description of Artificial Sequence: Syntheticpolypeptide
  VAR_SEQ 1: note = Gln or pyroglutamate
XVQLVQSGAEVKKPGASVKVSCKASGYTFASYSMHWVRQAPGQRLEWMGWINAGNWNTKYSQKFQGRVTITRDTSASTAYMELSSLRSEDTAVYYCARNWGMSYGMDVWGQGTTVTVSS

SEQ ID NO: 53 - protein, 119 aa, synthetic construct
  REGION 1..119: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAWTGNTKYSQKFQGRVTITRDTSASTAYMELSSLRSEDTAVYYCARNWGMSYGMDVWGQGTTVTVSS

SEQ ID NO: 54 - protein, 119 aa, synthetic construct
  REGION 1..119: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSTSTAYMELRSLRSDDTAMYYCARNWGMSYGLDVWGQGTMVTVSS

SEQ ID NO: 55 - protein, 119 aa, synthetic construct
  REGION 1..119: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSTSTAYMELRSLRSDDTAMYYCARNWGLSYGMDVWGQGTMVTVSS

SEQ ID NO: 56 - protein, 119 aa, synthetic construct
  REGION 1..119: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSTSTAYMELRSLRSDDTAMYYCARNWGLSYGLDVWGQGTMVTVSS

SEQ ID NO: 57 - protein, 119 aa, synthetic construct
  REGION 1..119: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARNWGMSYGLDVWGQGTTVTVSS

SEQ ID NO: 58 - protein, 119 aa, synthetic construct
  REGION 1..119: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARNWGLSYGMDVWGQGTTVTVSS

SEQ ID NO: 59 - protein, 119 aa, synthetic construct
  REGION 1..119: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARNWGLSYGLDVWGQGTTVTVSS

SEQ ID NO: 60 - protein, 118 aa, synthetic construct
  REGION 1..118: note = Description of Artificial Sequence: Syntheticpolypeptide
  VAR_SEQ 1: note = Gln or pyroglutamate
XVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARGYDSRPLDVWGQGTLVTVSS

SEQ ID NO: 61 - protein, 118 aa, synthetic construct
  REGION 1..118: note = Description of Artificial Sequence: Syntheticpolypeptide
  VAR_SEQ 1: note = Gln or pyroglutamate
XVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARGYDSRPLDYWGQGTLVTVSS

SEQ ID NO: 62 - protein, 108 aa, synthetic construct
  REGION 1..108: note = Description of Artificial Sequence: Syntheticpolypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPALTFGGGTKVDIK

SEQ ID NO: 63 - protein, 108 aa, synthetic construct
  REGION 1..108: note = Description of Artificial Sequence: Syntheticpolypeptide
DIQMTQSPSSLSASVGDRVTITCRASQSISSYLNWYQQKPGKAPKLLIYSASSLQSGVPSRFSGSGSGTDFTLTISSLQPEDFATYYCQQSYSTPALTFGGGTKVDIK

SEQ ID NO: 64 - protein, 108 aa, synthetic construct
  REGION 1..108: note = Description of Artificial Sequence: Syntheticpolypeptide
DIQMTQSPSSLSASVGDRVTITCRASQSISSYLNWYQQKPGKAPKLLIYSESSLQSGVPSRFSGSGSGTDFTLTISSLQPEDFATYYCQQSYSTPALTFGGGTKVDIK

SEQ ID NO: 65 - protein, 108 aa, synthetic construct
  REGION 1..108: note = Description of Artificial Sequence: Syntheticpolypeptide
DIQMTQSPSSLSASVGDRVTITCRASQSISSYLNWYQQKPGKAPKLLIYSSSSLQSGVPSRFSGSGSGTDFTLTISSLQPEDFATYYCQQSYSTPALTFGGGTKVDIK

SEQ ID NO: 66 - protein, 108 aa, synthetic construct
  REGION 1..108: note = Description of Artificial Sequence: Syntheticpolypeptide
DIQMTQSPSSLSASVGDRVTITCRASQSISPYLNWYQQKPGKAPKLLIYSASSLQSGVPSRFSGSGSGTDFTLTISSLQPEDFATYYCQQSYSTPALTFGGGTKVDIK

SEQ ID NO: 67 - protein, 108 aa, synthetic construct
  REGION 1..108: note = Description of Artificial Sequence: Syntheticpolypeptide
DIQMTQSPSSLSASVGDRVTITCRASQSISWYLNWYQQKPGKAPKLLIYSASSLQSGVPSRFSGSGSGTDFTLTISSLQPEDFATYYCQQSYSTPALTFGGGTKVDIK

SEQ ID NO: 68 - protein, 108 aa, synthetic construct
  REGION 1..108: note = Description of Artificial Sequence: Syntheticpolypeptide
DIQMTQSPSSLSASVGDRVTITCRASQSILSYLNWYQQKPGKAPKLLIYSASSLQSGVPSRFSGSGSGTDFTLTISSLQPEDFATYYCQQSYSTPALTFGGGTKVDIK

SEQ ID NO: 69 - protein, 108 aa, synthetic construct
  REGION 1..108: note = Description of Artificial Sequence: Syntheticpolypeptide
DIQMTQSPSSLSASVGDRVTITCRASQSITSYLNWYQQKPGKAPKLLIYAASSLQSGVPSRFSGSGSGTDFTLTISSLQPEDFATYYCQQAYSTPALSFGGGTKVDIK

SEQ ID NO: 70 - protein, 108 aa, synthetic construct
  REGION 1..108: note = Description of Artificial Sequence: Syntheticpolypeptide
SYELTQPLSVSVALGQTASITCGGNNIGSKIVHWYQQKSGQAPVLVVSDDRDRPSGIPERFSGSNSGNTATLTINTVEAGDEADYYCQVWDINVHHVIFGGGTKVTVL

SEQ ID NO: 71 - protein, 108 aa, synthetic construct
  REGION 1..108: note = Description of Artificial Sequence: Syntheticpolypeptide
SYELTQPLSVSVALGQTASITCGGNNIGSKIVHWYQQKPGQAPVLVVSDDRDRPSGIPERFSGSNSGNTATLTISRVEAGDEADYYCQVWDINVHHVIFGGGTKLTVL

SEQ ID NO: 72 - protein, 108 aa, synthetic construct
  REGION 1..108: note = Description of Artificial Sequence: Syntheticpolypeptide
SYELTQPLSVSVALGQTASITCGGNNIGSKIVHWYQQKSGQAPVLVIYDDRDRPSGIPERFSGSNSGNTATLTINTVEAGDEADYYCQVWDINVHHVIFGGGTKLTVL

SEQ ID NO: 73 - protein, 108 aa, synthetic construct
  REGION 1..108: note = Description of Artificial Sequence: Syntheticpolypeptide
SYELTQPLSVSVALGQTASITCGGNNIGSKIVHWYQQKPGQAPVLVVSDDRDRPSGIPERFSGSNSGNTATLTINTVEAGDEADYYCQVWDINVHHVIFGGGTKLTVL

SEQ ID NO: 74 - protein, 108 aa, synthetic construct
  REGION 1..108: note = Description of Artificial Sequence: Syntheticpolypeptide
SYELTQPLSVSVALGQTASITCGGNNIGSKIVHWYQQKSGQAPVLVVSDDRDRPSGIPERFSGSNSGNTATLTISRVEAGDEADYYCQVWDINVHHVIFGGGTKLTVL

SEQ ID NO: 75 - protein, 108 aa, synthetic construct
  REGION 1..108: note = Description of Artificial Sequence: Syntheticpolypeptide
SYELTQPLSVSVALGQTASITCGGNNIGSKIVHWYQQKSGQAPVLVVSDDRDRPSGIPERFSGSNSGNTATLTISRAQAGDEADYYCQVWDINVHHVIFGGGTKLTVL

SEQ ID NO: 76 - protein, 449 aa, synthetic construct
  REGION 1..449: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSTSTAYMELRSLRSDDTAMYYCARNWGMSYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 449 aa, synthetic construct
  REGION 1..449: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARNWGMSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 449 aa, synthetic construct
  REGION 1..449: note = Description of Artificial Sequence: Syntheticpolypeptide
  VAR_SEQ 1: note = Gln or pyroglutamate
XVQLVQSGAEVKKPGASVKVSCKASGYTFTQYAMHWVRQAPGQRLEWMGWINAVSGDTKYSQKFQGRVTITRDTSTSTAYMELRSLRSDDTAMYYCARNWGMSYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 449 aa, synthetic construct
  REGION 1..449: note = Description of Artificial Sequence: Syntheticpolypeptide
  VAR_SEQ 1: note = Gln or pyroglutamate
XVQLVQSGAEVKKPGASVKVSCKASGYTFTQYAMHWVRQAPGQRLEWMGWINAVSGDTKYSQKFQGRVTITRDTSASTAYMELSSLRSEDTAVYYCARNWGMSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 449 aa, synthetic construct
  REGION 1..449: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGTGDTKYSQKFQGRVTITRDTSTSTAYMELRSLRSDDTAMYYCARNWGMSYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 449 aa, synthetic construct
  REGION 1..449: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGTGDTKYSQKFQGRVTITRDTSASTAYMELSSLRSEDTAVYYCARNWGMSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 449 aa, synthetic construct
  REGION 1..449: note = Description of Artificial Sequence: Syntheticpolypeptide
  VAR_SEQ 1: note = Gln or pyroglutamate
XVQLVQSGAEVKKPGASVKVSCKASGYTFNQYAMHWVRQAPGQRLEWMGWINEGYGNTKYSQKFQGRVTITRDTSTSTAYMELRSLRSDDTAMYYCARNWGMSYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 449 aa, synthetic construct
  REGION 1..449: note = Description of Artificial Sequence: Syntheticpolypeptide
  VAR_SEQ 1: note = Gln or pyroglutamate
XVQLVQSGAEVKKPGASVKVSCKASGYTFTQYAMHWVRQAPGQRLEWMGWINAGYGYTKYSQKFQGRVTITRDTSTSTAYMELRSLRSDDTAMYYCARNWGMSYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 449 aa, synthetic construct
  REGION 1..449: note = Description of Artificial Sequence: Syntheticpolypeptide
  VAR_SEQ 1: note = Gln or pyroglutamate
XVQLVQSGAEVKKPGASVKVSCKASGYTFTQYAMHWVRQAPGQRLEWMGWINAGYGYTKYSQKFQGRVTITRDTSASTAYMELSSLRSEDTAVYYCARNWGMSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 449 aa, synthetic construct
  REGION 1..449: note = Description of Artificial Sequence: Syntheticpolypeptide
  VAR_SEQ 1: note = Gln or pyroglutamate
XVQLVQSGAEVKKPGASVKVSCKASGYTFSQYAMHWVRQAPGQRLEWMGWINAGTGNTKYSQKFQGRVTITRDTSTSTAYMELRSLRSDDTAMYYCARNWGMSYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 449 aa, synthetic construct
  REGION 1..449: note = Description of Artificial Sequence: Syntheticpolypeptide
  VAR_SEQ 1: note = Gln or pyroglutamate
XVQLVQSGAEVKKPGASVKVSCKASGYTFSQYAMHWVRQAPGQRLEWMGWINAGTGNTKYSQKFQGRVTITRDTSASTAYMELSSLRSEDTAVYYCARNWGMSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 449 aa, synthetic construct
  REGION 1..449: note = Description of Artificial Sequence: Syntheticpolypeptide
  VAR_SEQ 1: note = Gln or pyroglutamate
XVQLVQSGAEVKKPGASVKVSCKASGYTFDQYAMHWVRQAPGQRLEWMGWINAGYGNTKYSQKFQGRVTITRDTSTSTAYMELRSLRSDDTAMYYCARNWGMSYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 449 aa, synthetic construct
  REGION 1..449: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSTSTAYMELRSLRSDDTAVYYCARNWGMSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 449 aa, synthetic construct
  REGION 1..449: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAWNGNTKYSQKFQGRVTITRDTSASTAYMELSSLRSEDTAVYYCARNWGMSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 449 aa, synthetic construct
  REGION 1..449: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINVGTGTTKYSQKFQGRVTITRDTSASTAYMELSSLRSEDTAVYYCARNWGMSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 449 aa, synthetic construct
  REGION 1..449: note = Description of Artificial Sequence: Syntheticpolypeptide
  VAR_SEQ 1: note = Gln or pyroglutamate
XVQLVQSGAEVKKPGASVKVSCKASGYTFSSYAMHWVRQAPGQRLEWMGWINAVNGNTKYSQKFQGRVTITRDTSASTAYMELSSLRSEDTAVYYCARNWGMSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 449 aa, synthetic construct
  REGION 1..449: note = Description of Artificial Sequence: Syntheticpolypeptide
  VAR_SEQ 1: note = Gln or pyroglutamate
XVQLVQSGAEVKKPGASVKVSCKASGYTFASYSMHWVRQAPGQRLEWMGWINAGNWNTKYSQKFQGRVTITRDTSASTAYMELSSLRSEDTAVYYCARNWGMSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 449 aa, synthetic construct
  REGION 1..449: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAWTGNTKYSQKFQGRVTITRDTSASTAYMELSSLRSEDTAVYYCARNWGMSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 449 aa, synthetic construct
  REGION 1..449: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSTSTAYMELRSLRSDDTAMYYCARNWGMSYGLDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 449 aa, synthetic construct
  REGION 1..449: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSTSTAYMELRSLRSDDTAMYYCARNWGLSYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 449 aa, synthetic construct
  REGION 1..449: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSTSTAYMELRSLRSDDTAMYYCARNWGLSYGLDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 449 aa, synthetic construct
  REGION 1..449: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARNWGMSYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 449 aa, synthetic construct
  REGION 1..449: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARNWGLSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 449 aa, synthetic construct
  REGION 1..449: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARNWGLSYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARGYDSRPLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARGYDSRPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 215 aa, synthetic construct
  REGION 1..215: note = Description of Artificial Sequence: Syntheticpolypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPALTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 215 aa, synthetic construct
  REGION 1..215: note = Description of Artificial Sequence: Syntheticpolypeptide
DIQMTQSPSSLSASVGDRVTITCRASQSISSYLNWYQQKPGKAPKLLIYSASSLQSGVPSRFSGSGSGTDFTLTISSLQPEDFATYYCQQSYSTPALTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 215 aa, synthetic construct
  REGION 1..215: note = Description of Artificial Sequence: Syntheticpolypeptide
DIQMTQSPSSLSASVGDRVTITCRASQSISSYLNWYQQKPGKAPKLLIYSESSLQSGVPSRFSGSGSGTDFTLTISSLQPEDFATYYCQQSYSTPALTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 215 aa, synthetic construct
  REGION 1..215: note = Description of Artificial Sequence: Syntheticpolypeptide
DIQMTQSPSSLSASVGDRVTITCRASQSISSYLNWYQQKPGKAPKLLIYSSSSLQSGVPSRFSGSGSGTDFTLTISSLQPEDFATYYCQQSYSTPALTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 215 aa, synthetic construct
  REGION 1..215: note = Description of Artificial Sequence: Syntheticpolypeptide
DIQMTQSPSSLSASVGDRVTITCRASQSISPYLNWYQQKPGKAPKLLIYSASSLQSGVPSRFSGSGSGTDFTLTISSLQPEDFATYYCQQSYSTPALTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 215 aa, synthetic construct
  REGION 1..215: note = Description of Artificial Sequence: Syntheticpolypeptide
DIQMTQSPSSLSASVGDRVTITCRASQSISWYLNWYQQKPGKAPKLLIYSASSLQSGVPSRFSGSGSGTDFTLTISSLQPEDFATYYCQQSYSTPALTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 215 aa, synthetic construct
  REGION 1..215: note = Description of Artificial Sequence: Syntheticpolypeptide
DIQMTQSPSSLSASVGDRVTITCRASQSILSYLNWYQQKPGKAPKLLIYSASSLQSGVPSRFSGSGSGTDFTLTISSLQPEDFATYYCQQSYSTPALTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 215 aa, synthetic construct
  REGION 1..215: note = Description of Artificial Sequence: Syntheticpolypeptide
DIQMTQSPSSLSASVGDRVTITCRASQSITSYLNWYQQKPGKAPKLLIYAASSLQSGVPSRFSGSGSGTDFTLTISSLQPEDFATYYCQQAYSTPALSFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 214 aa, synthetic construct
  REGION 1..214: note = Description of Artificial Sequence: Syntheticpolypeptide
SYELTQPLSVSVALGQTASITCGGNNIGSKIVHWYQQKSGQAPVLVVSDDRDRPSGIPERFSGSNSGNTATLTINTVEAGDEADYYCQVWDINVHHVIFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 111 - protein, 214 aa, synthetic construct
  REGION 1..214: note = Description of Artificial Sequence: Syntheticpolypeptide
SYELTQPLSVSVALGQTASITCGGNNIGSKIVHWYQQKPGQAPVLVVSDDRDRPSGIPERFSGSNSGNTATLTISRVEAGDEADYYCQVWDINVHHVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 112 - protein, 214 aa, synthetic construct
  REGION 1..214: note = Description of Artificial Sequence: Syntheticpolypeptide
SYELTQPLSVSVALGQTASITCGGNNIGSKIVHWYQQKSGQAPVLVIYDDRDRPSGIPERFSGSNSGNTATLTINTVEAGDEADYYCQVWDINVHHVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 113 - protein, 214 aa, synthetic construct
  REGION 1..214: note = Description of Artificial Sequence: Syntheticpolypeptide
SYELTQPLSVSVALGQTASITCGGNNIGSKIVHWYQQKPGQAPVLVVSDDRDRPSGIPERFSGSNSGNTATLTINTVEAGDEADYYCQVWDINVHHVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 114 - protein, 214 aa, synthetic construct
  REGION 1..214: note = Description of Artificial Sequence: Syntheticpolypeptide
SYELTQPLSVSVALGQTASITCGGNNIGSKIVHWYQQKSGQAPVLVVSDDRDRPSGIPERFSGSNSGNTATLTISRVEAGDEADYYCQVWDINVHHVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 115 - protein, 214 aa, synthetic construct
  REGION 1..214: note = Description of Artificial Sequence: Syntheticpolypeptide
SYELTQPLSVSVALGQTASITCGGNNIGSKIVHWYQQKSGQAPVLVVSDDRDRPSGIPERFSGSNSGNTATLTISRAQAGDEADYYCQVWDINVHHVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 116 - protein, 449 aa, synthetic construct
  REGION 1..449: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSTSTAYMELRSLRSDDTAMYYCARNWGMSYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARGYDSRPLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 449 aa, synthetic construct
  REGION 1..449: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSTSTAYMELRSLRSDDTAMYYCARNWGMSYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARGYDSRPLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 449 aa, synthetic construct
  REGION 1..449: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSTSTAYMELRSLRSDDTAMYYCARNWGMSYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 107 aa, synthetic construct [sequence omitted]

SEQ ID NO: 123 - protein, 106 aa, synthetic construct
  REGION 1..106: note = Description of Artificial Sequence: Syntheticpolypeptide
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 124 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 330 aa, synthetic construct
  REGION 1..330: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 498 aa, Homo sapiens
VWEKTVNTEENVYATLGSDVNLTCQTQTVGFFVQMQWSKVTNKIDLIAVYHPQYGFYCAYGRPCESLVTFTETPENGSKWTLHLRNMSCSVSGRYECMLVLYPEGIQTKIYNLLIQTHVTADEWNSNHTIEIEINQTLEIPCFQNSSSKISSEFTYAWSVENSSTDSWVLLSKGIKEDNGTQETLISQNHLISNSTLLKDRVKLGTDYRLHLSPVQIFDDGRKFSCHIRVGPNKILRSSTTVKVFAKPEIPVIVENNSTDVLVERRFTCLLKNVFPKANITWFIDGSFLHDEKEGIYITNEERKGKDGFLELKSVLTRVHSNKPAQSDNLTIWCMALSPVPGNKVWNISSEKITFLLGSEISSTDPPLSVTESTLDTQPSPASSVSPARYPATSSVTLVDVSALRPNTTPQPSNSSMTTRGFNYPWTSSGTDTKKSVSRIPSETYSSSPSGAGSTLHDNVFTSTARAFSEVPTTANGSTKTNHVHITGIVVNKPKDGM

SEQ ID NO: 128 - protein, 482 aa, Homo sapiens
VWEKTVNTEENVYATLGSDVNLTCQTQTVGFFVQMQWSKVTNKIDLIAVYHPQYGFYCAYGRPCESLVTFTETPENGSKWTLHLRNMSCSVSGRYECMLVLYPEGIQTKIYNLLIQTHVTADEWNSNHTIEIEINQTLEIPCFQNSSSKISSEFTYAWSVEDNGTQETLISQNHLISNSTLLKDRVKLGTDYRLHLSPVQIFDDGRKFSCHIRVGPNKILRSSTTVKVFAKPEIPVIVENNSTDVLVERRFTCLLKNVFPKANITWFIDGSFLHDEKEGIYITNEERKGKDGFLELKSVLTRVHSNKPAQSDNLTIWCMALSPVPGNKVWNISSEKITFLLGSEISSTDPPLSVTESTLDTQPSPASSVSPARYPATSSVTLVDVSALRPNTTPQPSNSSMTTRGFNYPWTSSGTDTKKSVSRIPSETYSSSPSGAGSTLHDNVFTSTARAFSEVPTTANGSTKTNHVHITGIVVNKPKDGM

SEQ ID NO: 129 - protein, 482 aa, Homo sapiens
VWEKTVNTEENVYATLGSDVNLTCQTQTVGFFVQMQWSKVTNKIDLIAVYHPQYGFYCAYGRPCESLVTFTETPENGSKWTLHLRNMSSSVSGRYECMLVLYPEGIQTKIYNLLIQTHVTADEWNSNHTIEIEINQTLEIPCFQNSSSKISSEFTYAWSVEDNGTQETLISQNHLISNSTLLKDRVKLGTDYRLHLSPVQIFDDGRKFSCHIRVGPNKILRSSTTVKVFAKPEIPVIVENNSTDVLVERRFTCLLKNVFPKANITWFIDGSFLHDEKEGIYITNEERKGKDGFLELKSVLTRVHSNKPAQSDNLTIWCMALSPVPGNKVWNISSEKITFLLGSEISSTDPPLSVTESTLDTQPSPASSVSPARYPATSSVTLVDVSALRPNTTPQPSNSSMTTRGFNYPWTSSGTDTKKSVSRIPSETYSSSPSGAGSTLHDNVFTSTARAFSEVPTTANGSTKTNHVHITGIVVNKPKDGM

SEQ ID NO: 130 - protein, 119 aa, Homo sapiens
VWEKTVNTEENVYATLGSDVNLTCQTQTVGFFVQMQWSKVTNKIDLIAVYHPQYGFYCAYGRPCESLVTFTETPENGSKWTLHLRNMSCSVSGRYECMLVLYPEGIQTKIYNLLIQTHV

SEQ ID NO: 131 - protein, 119 aa, Homo sapiens
VWEKTVNTEENVYATLGSDVNLTCQTQTVGFFVQMQWSKVTNKIDLIAVYHPQYGFYCAYGRPCESLVTFTETPENGSKWTLHLRNMSSSVSGRYECMLVLYPEGIQTKIYNLLIQTHV

SEQ ID NO: 132 - protein, 499 aa, Macaca fascicularis
VWGKPFNTEENIYATLGSDVNLTCQTQAKGFLVQMQWSKVTDKADLIALYHPQYGFHCAYGSPCESLVTFTQTPENGSKWTLHLRNMSSSVSGRYECMLTLYPEGMQTKIYNLLIQTHVTPDEWKSNHTIEIEINQTLEIPCFQNSSSEISSEFTYAWLVVKNSSTDSWVLLSKGKRYDNGTQQTLISQDHLISSSTLLKDRVKVGIDYRLHLSPVQIFDDGRKFSCHIRVGPDKILRSSTTIKVFAKPEIPMIVENNSTDVLVERTFTCLLKNVFPKANIIWFIDGSFLHDEKEGIYITNEERKGKDGFLELKSVLTRVHSDKPAQSDNLTIWCMALSPVPGNKVWNISSEKITFLLGSEMSTTDLPPSVTESTLDTQPSPASSVSPTRYPATSSVTLADVSALRPNTTPQSSSSSVTTQDFNYPWTSSGTDAKKSFSQIPSETYSSSPSGAGSTLHDNVFTSTTRALSEVPTTANGSTKTNHVHITGIVVSKPKDGM

SEQ ID NO: 133 - protein, 482 aa, Macaca fascicularis
VWGKPFNTEENIYATLGSDVNLTCQTQAKGFLVQMQWSKVTDKADLIALYHPQYGFHCAYGSPCESLVTFTQTPENGSKWTLHLRNMSSSVSGRYECMLTLYPEGMQTKIYNLLIQTHVTPDEWKSNHTIEIEINQTLEIPCFQNSSSEISSEFTYAWLVEDNGTQQTLISQDHLISSSTLLKDRVKVGIDYRLHLSPVQIFDDGRKFSCHIRVGPDKILRSSTTIKVFAKPEIPMIVENNSTDVLVERTFTCLLKNVFPKANIIWFIDGSFLHDEKEGIYITNEERKGKDGFLELKSVLTRVHSDKPAQSDNLTIWCMALSPVPGNKVWNISSEKITFLLGSEMSTTDLPPSVTESTLDTQPSPASSVSPTRYPATSSVTLADVSALRPNTTPQSSSSSVTTQDFNYPWTSSGTDAKKSFSQIPSETYSSSPSGAGSTLHDNVFTSTTRALSEVPTTANGSTKTNHVHITGIVVSKPKDGM

SEQ ID NO: 134 - protein, 119 aa, Macaca fascicularis
VWGKPFNTEENIYATLGSDVNLTCQTQAKGFLVQMQWSKVTDKADLIALYHPQYGFHCAYGSPCESLVTFTQTPENGSKWTLHLRNMSSSVSGRYECMLTLYPEGMQTKIYNLLIQTHV

SEQ ID NO: 135-136 - skipped (2 entries)

SEQ ID NO: 137 - protein, 17 aa, synthetic construct
  REGION 1..17: note = Description of Artificial Sequence: Syntheticpeptide
  VAR_SEQ 1: note = Trp or Gly
  VAR_SEQ 3: note = Asn or Ile
  VAR_SEQ 4: note = Ala, Glu, Val, or Pro
  VAR_SEQ 5: note = Val, Gly, Trp, or Ile
  VAR_SEQ 6: note = Ser, Tyr, Thr, Asn, or Phe
  VAR_SEQ 7: note = Gly or Trp
  VAR_SEQ 8: note = Asp, Tyr, Asn, or Thr
  VAR_SEQ 9: note = Thr or Ala
  VAR_SEQ 10: note = Lys or Asn
  VAR_SEQ 12: note = Ser or Ala
XIXXXXXXXXYXQKFQG

SEQ ID NO: 138 - protein, 17 aa, synthetic construct
  REGION 1..17: note = Description of Artificial Sequence: Syntheticpeptide
  VAR_SEQ 4: note = Ala, Val, or Glu
  VAR_SEQ 5: note = Val, Trp, or Gly
  VAR_SEQ 6: note = Ser, Tyr, Thr, or Asn
  VAR_SEQ 7: note = Gly or Trp
  VAR_SEQ 8: note = Asp, Asn, Tyr, or Thr
WINXXXXXTKYSQKFQG

SEQ ID NO: 139 - protein, 11 aa, synthetic construct
  REGION 1..11: note = Description of Artificial Sequence: Syntheticpeptide
  VAR_SEQ 7: note = Ser, Thr, or Leu
  VAR_SEQ 8: note = Ser, Pro, or Trp
RASQSIXXYLN

SEQ ID NO: 140 - protein, 10 aa, synthetic construct
  REGION 1..10: note = Description of Artificial Sequence: Syntheticpeptide
NWGMSYGLDV

SEQ ID NO: 141 - protein, 7 aa, synthetic construct
  REGION 1..7: note = Description of Artificial Sequence: Syntheticpeptide
  VAR_SEQ 1: note = Ser or Ala
  VAR_SEQ 2: note = Ala, Ser, or Glu
XXSSLQS

SEQ ID NO: 142 - protein, 10 aa, synthetic construct
  REGION 1..10: note = Description of Artificial Sequence: Syntheticpeptide
NWGLSYGMDV

SEQ ID NO: 143 - protein, 10 aa, synthetic construct
  REGION 1..10: note = Description of Artificial Sequence: Syntheticpeptide
  VAR_SEQ 3: note = Ser or Ala
  VAR_SEQ 10: note = Thr or Ser
QQXYSTPALX

SEQ ID NO: 144 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSTSTAYMELRSLRSDDTAMYYCARNWGMSYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 145 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARNWGMSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 146 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGASVKVSCKASGYTFTQYAMHWVRQAPGQRLEWMGWINAVSGDTKYSQKFQGRVTITRDTSTSTAYMELRSLRSDDTAMYYCARNWGMSYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 147 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGASVKVSCKASGYTFTQYAMHWVRQAPGQRLEWMGWINAVSGDTKYSQKFQGRVTITRDTSASTAYMELSSLRSEDTAVYYCARNWGMSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 148 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGTGDTKYSQKFQGRVTITRDTSTSTAYMELRSLRSDDTAMYYCARNWGMSYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 149 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGTGDTKYSQKFQGRVTITRDTSASTAYMELSSLRSEDTAVYYCARNWGMSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 150 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGASVKVSCKASGYTFNQYAMHWVRQAPGQRLEWMGWINEGYGNTKYSQKFQGRVTITRDTSTSTAYMELRSLRSDDTAMYYCARNWGMSYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 151 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGASVKVSCKASGYTFTQYAMHWVRQAPGQRLEWMGWINAGYGYTKYSQKFQGRVTITRDTSTSTAYMELRSLRSDDTAMYYCARNWGMSYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 152 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGASVKVSCKASGYTFTQYAMHWVRQAPGQRLEWMGWINAGYGYTKYSQKFQGRVTITRDTSASTAYMELSSLRSEDTAVYYCARNWGMSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 153 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGASVKVSCKASGYTFSQYAMHWVRQAPGQRLEWMGWINAGTGNTKYSQKFQGRVTITRDTSTSTAYMELRSLRSDDTAMYYCARNWGMSYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 154 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGASVKVSCKASGYTFSQYAMHWVRQAPGQRLEWMGWINAGTGNTKYSQKFQGRVTITRDTSASTAYMELSSLRSEDTAVYYCARNWGMSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 155 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGASVKVSCKASGYTFDQYAMHWVRQAPGQRLEWMGWINAGYGNTKYSQKFQGRVTITRDTSTSTAYMELRSLRSDDTAMYYCARNWGMSYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 156 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSTSTAYMELRSLRSDDTAVYYCARNWGMSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 157 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAWNGNTKYSQKFQGRVTITRDTSASTAYMELSSLRSEDTAVYYCARNWGMSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 158 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINVGTGTTKYSQKFQGRVTITRDTSASTAYMELSSLRSEDTAVYYCARNWGMSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 159 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGASVKVSCKASGYTFSSYAMHWVRQAPGQRLEWMGWINAVNGNTKYSQKFQGRVTITRDTSASTAYMELSSLRSEDTAVYYCARNWGMSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 160 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGASVKVSCKASGYTFASYSMHWVRQAPGQRLEWMGWINAGNWNTKYSQKFQGRVTITRDTSASTAYMELSSLRSEDTAVYYCARNWGMSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 161 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAWTGNTKYSQKFQGRVTITRDTSASTAYMELSSLRSEDTAVYYCARNWGMSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 162 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSTSTAYMELRSLRSDDTAMYYCARNWGMSYGLDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 163 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSTSTAYMELRSLRSDDTAMYYCARNWGLSYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 164 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSTSTAYMELRSLRSDDTAMYYCARNWGLSYGLDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 165 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARNWGMSYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 166 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARNWGLSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 167 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARNWGLSYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 168 - protein, 447 aa, synthetic construct
  REGION 1..447: note = Description of Artificial Sequence: Syntheticpolypeptide
  VAR_SEQ 1: note = Gln or pyroglutamate
XVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARGYDSRPLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 169 - protein, 447 aa, synthetic construct
  REGION 1..447: note = Description of Artificial Sequence: Syntheticpolypeptide
  VAR_SEQ 1: note = Gln or pyroglutamate
XVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARGYDSRPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 170 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSTSTAYMELRSLRSDDTAMYYCARNWGMSYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 171 - protein, 447 aa, synthetic construct
  REGION 1..447: note = Description of Artificial Sequence: Syntheticpolypeptide
  VAR_SEQ 1: note = Gln or pyroglutamate
XVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARGYDSRPLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 172 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSTSTAYMELRSLRSDDTAMYYCARNWGMSYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 173 - protein, 447 aa, synthetic construct
  REGION 1..447: note = Description of Artificial Sequence: Syntheticpolypeptide
  VAR_SEQ 1: note = Gln or pyroglutamate
XVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARGYDSRPLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 174 - protein, 448 aa, synthetic construct
  REGION 1..448: note = Description of Artificial Sequence: Syntheticpolypeptide
  VAR_SEQ 1: note = Gln or pyroglutamate
XVQLVQSGAEVKKPGASVKVSCKASGYTFTSYAMHWVRQAPGQRLEWMGWINAGNGNTKYSQKFQGRVTITRDTSTSTAYMELRSLRSDDTAMYYCARNWGMSYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 175 - protein, 329 aa, synthetic construct
  REGION 1..329: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 176 - protein, 329 aa, synthetic construct
  REGION 1..329: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 177 - protein, 329 aa, synthetic construct
  REGION 1..329: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 178 - protein, 329 aa, synthetic construct
  REGION 1..329: note = Description of Artificial Sequence: Syntheticpolypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 179 - protein, 10 aa, synthetic construct
  REGION 1..10: note = Description of Artificial Sequence: Syntheticpeptide
NWGLSYGLDV

SEQ ID NO: 180 - protein, 10 aa, synthetic construct
  REGION 1..10: note = Description of Artificial Sequence: Syntheticpeptide
  VAR_SEQ 4: note = Met or Leu
  VAR_SEQ 8: note = Met or Leu
NWGXSYGXDV

(19) United States
(12) Patent Application Publication
(10) Pub. No.: US 2024/0209082 A1
(43) Pub. Date: Jun. 27, 2024

(54) ANTI-CD96 ANTIBODIES AND METHODS OF USE THEREOF

(71) Applicant: AGENUS INC., Lexington, MA (US)

(72) Inventors: Dhan Sidhartha CHAND, Woburn, MA (US); Randi Barbara GOMBOS, Wilmington, MA (US); Olga IGNATOVICH, Cambridge (GB); Nicola Anne RAMSAY, Cambridge (GB); K. Mark BUSHELL, SAFFRON WALDEN (GB); Emmanuel Cyrille Pascal BRIEND, Cambridge (GB)

(21) Appl. No.: 18/313,858

(22) Filed: May 8, 2023

Related U.S. Application Data
(62) Division of application No. 17/007,238, filed on Aug. 31, 2020, now Pat. No. 11,680,098.
(60) Provisional application No. 62/931,476, filed on Nov. 6, 2019.
(60) Provisional application No. 62/894,334, filed on Aug. 30, 2019.

(51) Int. Cl. C07K 16/28; A61K 31/713; A61K 39/00; A61K 45/06; A61K 47/68; C12N 5/10; C12N 15/63

(52) U.S. Cl. CPC C07K 16/2803; A61K 31/713; A61K 39/001129; A61K 47/6803; A61K 47/6849; C12N 5/10; C12N 15/63; A61K 2039/507; A61K 45/06; C07K 2317/21; C07K 2317/31; C07K 2317/565

(57) ABSTRACT

The instant disclosure provides antibodies that specifically bind to CD96 (e.g., human CD96) and antagonize CD96 function. Also provided are pharmaceutical compositions comprising these antibodies, nucleic acids encoding these antibodies, expression vectors and host cells for making these antibodies, and methods of treating a subject using these antibodies.

### RELATED APPLICATIONS

This application is a divisional of U.S. application Ser. No. 17/007,238, filed Aug. 31, 2020, issued as U.S. Pat. No. 11,680,098, on Jun. 20, 2023, which claims the benefit of U.S. Provisional Application No. 62/894,334, filed Aug. 30, 2019, and 62/931,476, filed Nov. 6, 2019, each of which is incorporated by reference herein in its entirety.

### SEQUENCE LISTING

The instant application contains a sequence listing which has been submitted electronically in XML ST.26 format and is hereby incorporated by reference in its entirety (said XML, ST.26 copy, created on Sep. 8, 2023, is named 404306-AGBW-144USD1 (199348) Sequence Listing.xml and is 307,160 bytes in size).

### 1. FIELD

The instant disclosure relates to antibodies that specifically bind to CD96 (e.g., human CD96) and methods of using the same.

### 2. BACKGROUND

CD96 (Cluster of Differentiation 96), also known as TACTILE (T cell-activation, increased late expression), is a type I transmembrane protein in the immunoglobulin (Ig) superfamily. It has a single Ig domain, a type I transmembrane domain, a single intracellular immunoreceptor tyrosine-based inhibitory motif (ITIM), and a single YXXM phosphorylation motif, and is expressed on the surface of T cells and natural killer (NK) cells.

CD96 is believed to play a role in the regulation of immune cells (e.g., NK cells and T cells) and tumor metastasis. In particular, it has been shown that blockade of CD96 function suppressed primary tumor growth in several mouse tumor models in a CD8+ T cell-dependent manner.

Given the role of human CD96 in modulating immune responses, therapeutic agents designed to block CD96 ligand interactions hold great promise for the treatment of diseases that involve immune suppression.

### 3. SUMMARY

The instant disclosure provides antibodies that specifically bind to CD96 (e.g., human CD96) and modulate CD96 function, e.g., CD96-mediated immune suppression. Also provided are pharmaceutical compositions comprising these antibodies, nucleic acids encoding these antibodies, expression vectors and host cells for making these antibodies, and methods of treating a subject using these antibodies. The antibodies disclosed herein are particularly useful for increasing immune cell activation, and hence, are useful for treating cancer in a subject or treating or preventing an infectious disease in a subject.

Accordingly, in one aspect, the instant disclosure provides an isolated antibody that specifically binds to human CD96, the antibody comprising a heavy chain variable region (VH) comprising complementarity determining regions (CDRs) CDRH1, CDRH2, and CDRH3, and a light chain variable region (VL) comprising CDRs CDRL1, CDRL2, and CDRL3, wherein:
(a) CDRH1 comprises the amino acid sequence of X₁YX₂X₃X₄ (SEQ ID NO: 135), wherein
   X₁ is Q or S;
   X₂ is A or S;
   X₃ is M or I; and
   X₄ is H or S;
(b) CDRH2 comprises the amino acid sequence of X₁IX₂X₃X₄X₅X₆X₇X₈X₉YX₁₀QKFQG (SEQ ID NO: 137), wherein
   X₁ is W or G;
   X₂ is N or I;
   X₃ is A, E, V, or P;
   X₄ is V, G, W, or I;
   X₅ is S, Y, T, N, or F;
   X₆ is G or W;
   X₇ is D, Y, N, or T;
   X₈ is T or A;
   X₉ is K or N; and
   X₁₀ is S or A;
(c) CDRH3 comprises the amino acid sequence of NWGX₁SYGX₂DV (SEQ ID NO: 180), GYDSRPLDV (SEQ ID NO: 19), or GYDSRPLDY (SEQ ID NO: 20), wherein
   X₁ is M or L; and
   X₂ is M or L;
(d) CDRL1 comprises the amino acid sequence of RASQSIX₁X₂YLN (SEQ ID NO: 139) or GGNNIGSKIVH (SEQ ID NO: 26), wherein
   X₁ is S, T, or L; and
   X₂ is S, P, or W;
(e) CDRL2 comprises the amino acid sequence of X₁X₂SSLQS (SEQ ID NO: 141) or DDRDRPS (SEQ ID NO: 32), wherein
   X₁ is S or A; and
   X₂ is A, S, or E; and/or
(f) CDRL3 comprises the amino acid sequence of QQX₁YSTPALX₂ (SEQ ID NO: 143) or QVWDINVHHVI (SEQ ID NO: 35), wherein
   X₁ is S or A; and
   X₂ is T or S, optionally wherein the amino acid immediately N-terminal to CDRH1 is N, T, S, D, or A.

In certain embodiments:
(a) CDRH1 comprises the amino acid sequence of X₁YX₂MH (SEQ ID NO: 136), wherein
   X₁ is Q or S; and
   X₂ is A or S;
(b) CDRH2 comprises the amino acid sequence of WINX₁X₂X₃X₄XsTKYSQKFQG (SEQ ID NO: 138), wherein
   X₁ is A, V, or E;
   X₂ is V, W, or G;
   X₃ is S, Y, T, or N;
   X₄ is G or W; and
   X₅ is D, N, Y, or T;
(c) CDRH3 comprises the amino acid sequence of NWGX₁SYGX₂DV (SEQ ID NO: 180), wherein
   X₁ is M or L; and
   X₂ is M or L;
(d) CDRL1 comprises the amino acid sequence of RASQSIX₁X₂YLN (SEQ ID NO: 139), wherein
   X₁ is S, T, or L; and
   X₂ is S, P, or W;
(e) CDRL2 comprises the amino acid sequence of X₁X₂SSLQS (SEQ ID NO: 141), wherein
   X₁ is S or A; and
   X₂ is A, S, or E; and/or
(f) CDRL3 comprises the amino acid sequence of QQSYSTPALT (SEQ ID NO: 33) or QQAYSTPALS (SEQ ID NO: 34).

In certain embodiments:
(a) CDRH1 comprises the amino acid sequence of SEQ ID NO: 4;
(b) CDRH2 comprises the amino acid sequence of SEQ ID NO: 17;
(c) CDRH3 comprises the amino acid sequence of SEQ ID NO: 19 or 20;
(d) CDRL1 comprises the amino acid sequence of SEQ ID NO: 26;
(e) CDRL2 comprises the amino acid sequence of SEQ ID NO: 32; and/or
(f) CDRL3 comprises the amino acid sequence of SEQ ID NO: 35.

In certain embodiments, CDRH1, CDRH2, and CDRH3 comprise the amino acid sequences of SEQ ID NOs: 1, 5, and 18; 2, 6, and 18; 2, 8, and 18; 2, 9, and 18; 2, 10, and 18; 1, 7, and 18; 2, 11, and 18; 1, 12, and 18; 1, 13, and 18; 1, 14, and 18; 3, 15, and 18; 1, 16, and 18; 1, 5, and 140; 1, 5, and 142; 1, 5, and 179; 4, 17, and 19; or 4, 17, and 20, respectively.

In certain embodiments, CDRL1, CDRL2, and CDRL3 comprise the amino acid sequences of SEQ ID NOs: 21, 28, and 33; 21, 29, and 33; 21, 30, and 33; 21, 31, and 33; 22, 29, and 33; 24, 29, and 33; 23, 29, and 33; 25, 28, and 34; or 26, 32, and 35, respectively.

In certain embodiments, CDRH1, CDRH2, CDRH3, CDRL1, CDRL2, and CDRL3 comprise the amino acid sequences of SEQ ID NOs: 1, 5, 18, 21, 28, and 33; 1, 5, 18, 21, 29, and 33; 1, 5, 18, 22, 29, and 33; 1, 5, 18, 23, 29, and 33; 1, 5, 18, 24, 29, and 33; 1, 5, 18, 25, 28, and 34; 1, 5, 140, 21, 28, and 33; 1, 5, 142, 21, 28, and 33; 1, 5, 179, 21, 28, and 33; 1, 7, 18, 21, 29, and 33; 1, 12, 18, 21, 28, and 33; 1, 13, 18, 21, 28, and 33; 1, 14, 18, 21, 28, and 33; 1, 16, 18, 21, 28, and 33; 2, 6, 18, 21, 29, and 33; 2, 8, 18, 21, 29, and 33; 2, 9, 18, 21, 30, and 33; 2, 10, 18, 21, 29, and 33; 2, 11, 18, 21, 31, and 33; 3, 15, 18, 21, 28, and 33;4, 17, 19, 26, 32, and 35; or 4, 17, 20, 26, 32, and 35, respectively.

In certain embodiments, the antibody comprises a VH comprising an amino acid sequence that is at least 75%, 80%, 85%, 90%, 95%, 99%, or 100% identical to the amino acid sequence of SEQ ID NO: 36, 37, 38, 39, 40, 41, 42, 43, 44, 45, 46, 47, 48, 49, 50, 51, 52, 53, 54, 55, 56, 57, 58, 59, 60, or 61. In certain embodiments, the amino acid sequence of the VH consists of the amino acid sequence of SEQ ID NO: 36, 37, 38, 39, 40, 41, 42, 43, 44, 45, 46, 47, 48, 49, 50, 51, 52, 53, 54, 55, 56, 57, 58, 59, 60, or 61. In certain embodiments, the X in any one of SEQ ID NOs: 36, 37, 38, 39, 40, 41, 42, 43, 44, 45, 46, 47, 48, 49, 50, 51, 52, 53, 54, 55, 56, 57, 58, 59, 60, or 61 is glutamine. In certain embodiments, the X in any one of SEQ ID NOs: 36, 37, 38, 39, 40, 41, 42, 43, 44, 45, 46, 47, 48, 49, 50, 51, 52, 53, 54, 55, 56, 57, 58, 59, 60, or 61 is pyroglutamate.

In certain embodiments, the antibody comprises a VL comprising an amino acid sequence that is at least 75%, 80%, 85%, 90%, 95%, 99%, or 100% identical to the amino acid sequence of SEQ ID NO: 62, 63, 64, 65, 66, 67, 68, 69, 70, 71, 72, 73, 74, or 75. In certain embodiments, the amino acid sequence of the VL consists of the amino acid sequence of SEQ ID NO: 62, 63, 64, 65, 66, 67, 68, 69, 70, 71, 72, 73, 74, or 75.

In another aspect, the instant disclosure provides an isolated antibody that specifically binds to human CD96, the antibody comprising: a VH comprising the amino acid sequence of SEQ ID NO: 36, 37, 38, 39, 40, 41, 42, 43, 44, 45, 46, 47, 48, 49, 50, 51, 52, 53, 54, 55, 56, 57, 58, 59, 60, or 61; and/or a VL comprising the amino acid sequence of SEQ ID NO: 62, 63, 64, 65, 66, 67, 68, 69, 70, 71, 72, 73, 74, or 75. In certain embodiments, the VH and VL comprise the amino acid sequences of SEQ ID NOs: 36 and 62; 37 and 62; 37 and 63; 37 and 66; 37 and 67; 37 and 68; 37 and 69; 38 and 63; 39 and 63; 40 and 63; 41 and 63; 42 and 63; 43 and 64; 44 and 64; 45 and 63; 46 and 63; 47 and 65; 48 and 62; 49 and 62; 50 and 62; 51 and 62; 52 and 62; 53 and 62; 54 and 62; 55 and 62; 56 and 62; 57 and 62; 58 and 62; 59 and 62; 60 and 70; 60 and 71; 60 and 72; 60 and 73; 60 and 74; 60 and 75; or 61 and 70, respectively. In certain embodiments, the amino acid sequences of the VH and VL consist of the amino acid sequences of SEQ ID NOs: 36 and 62; 37 and 62; 37 and 63; 37 and 66; 37 and 67; 37 and 68; 37 and 69; 38 and 63; 39 and 63; 40 and 63; 41 and 63; 42 and 63; 43 and 64; 44 and 64; 45 and 63; 46 and 63; 47 and 65; 48 and 62; 49 and 62; 50 and 62; 51 and 62; 52 and 62; 53 and 62; 54 and 62; 55 and 62; 56 and 62; 57 and 62; 58 and 62; 59 and 62; 60 and 70; 60 and 71; 60 and 72; 60 and 73; 60 and 74; 60 and 75; or 61 and 70, respectively. In certain embodiments, the X in any one of SEQ ID NOs: 36, 37, 38, 39, 40, 41, 42, 43, 44, 45, 46, 47, 48, 49, 50, 51, 52, 53, 54, 55, 56, 57, 58, 59, 60, or 61 is glutamine. In certain embodiments, the X in any one of SEQ ID NOs: 36, 37, 38, 39, 40, 41, 42, 43, 44, 45, 46, 47, 48, 49, 50, 51, 52, 53, 54, 55, 56, 57, 58, 59, 60, or 61 is pyroglutamate.

In certain embodiments, the antibody specifically binds to the amino acid sequence of SEQ ID NO: 130 or 131. In certain embodiments, the antibody binds to the amino acid sequence of SEQ ID NO: 134.

In certain embodiments, the antibody is internalized upon binding to cells expressing human CD96.

In another aspect, the instant disclosure provides an isolated antibody that specifically binds the amino acid sequence of SEQ ID NO: 130 or 131. In certain embodiments, the antibody binds to the amino acid sequence of SEQ ID NO: 134.

In another aspect, the instant disclosure provides an isolated antibody that specifically binds to human CD96, wherein the antibody is internalized upon binding to cells expressing human CD96.

In certain embodiments, the antibody comprises a heavy chain constant region selected from the group consisting of human IgG1, IgG2, IgG3, IgG4, IgA1, and IgA2. In certain embodiments, the antibody comprises an IgG1 heavy chain constant region. In certain embodiments, the amino acid sequence of the IgG1 heavy chain constant region comprises an N297A mutation, numbered according to the EU numbering system. In certain embodiments, the antibody comprises a heavy chain constant region comprising the amino acid sequence of SEQ ID NO: 124 or 176. In certain embodiments, the amino acid sequence of the IgG1 heavy chain constant region comprises S239D, A330L, and I332E mutations, numbered according to the EU numbering system. In certain embodiments, the antibody comprises a heavy chain constant region comprising the amino acid sequence of SEQ ID NO: 125 or 177. In certain embodiments, the amino acid sequence of the IgG1 heavy chain constant region comprises S267E and L328F mutations, numbered according to the EU numbering system. In certain embodiments, the antibody comprises a heavy chain constant region comprising the amino acid sequence of SEQ ID NO: 126 or 178. In certain embodiments, the antibody comprises a heavy chain constant region that is a variant of a wild type heavy chain constant region, wherein the variant heavy chain constant region binds to an FcγR with higher affinity than the wild type heavy chain constant region binds to the FcγR. In certain embodiments, the FcγR is FcγRIIB.

In certain embodiments, the antibody comprises a heavy chain comprising the amino acid sequence of SEQ ID NO: 76, 77, 78, 79, 80, 81, 82, 83, 84, 85, 86, 87, 88, 89, 90, 91, 92, 93, 94, 95, 96, 97, 98, 99, 100, 101, 144, 145, 146, 147, 148, 149, 150, 151, 152, 153, 154, 155, 156, 157, 158, 159, 160, 161, 162, 163, 164, 165, 166, 167, 168, or 169. In certain embodiments, the amino acid sequence of the heavy chain consists of the amino acid sequence of SEQ ID NO: 76, 77, 78, 79, 80, 81, 82, 83, 84, 85, 86, 87, 88, 89, 90, 91, 92, 93, 94, 95, 96, 97, 98, 99, 100, 101, 144, 145, 146, 147, 148, 149, 150, 151, 152, 153, 154, 155, 156, 157, 158, 159, 160, 161, 162, 163, 164, 165, 166, 167, 168, or 169. In certain embodiments, the X in any one of SEQ ID NOs: 76, 77, 78, 79, 80, 81, 82, 83, 84, 85, 86, 87, 88, 89, 90, 91, 92, 93, 94, 95, 96, 97, 98, 99, 100, 101, 144, 145, 146, 147, 148, 149, 150, 151, 152, 153, 154, 155, 156, 157, 158, 159, 160, 161, 162, 163, 164, 165, 166, 167, 168, and 169 is glutamine. In certain embodiments, the X in any one of SEQ ID NOs: 76, 77, 78, 79, 80, 81, 82, 83, 84, 85, 86, 87, 88, 89, 90, 91, 92, 93, 94, 95, 96, 97, 98, 99, 100, 101, 144, 145, 146, 147, 148, 149, 150, 151, 152, 153, 154, 155, 156, 157, 158, 159, 160, 161, 162, 163, 164, 165, 166, 167, 168, and 169 is pyroglutamate.

In certain embodiments, the antibody comprises a light chain constant region comprising the amino acid sequence of SEQ ID NO: 122 or 123. In certain embodiments, the antibody comprises a light chain comprising the amino acid sequence of SEQ ID NO: 102, 103, 104, 105, 106, 107, 108, 109, 110, 111, 112, 113, 114, or 115. In certain embodiments, the amino acid sequence of the light chain consists of the amino acid sequence of SEQ ID NO: 102, 103, 104, 105, 106, 107, 108, 109, 110, 111, 112, 113, 114, or 115.

In another aspect, the instant disclosure provides an isolated antibody that specifically binds to human CD96, the antibody comprising: a heavy chain comprising the amino acid sequence of SEQ ID NO: 76, 77, 78, 79, 80, 81, 82, 83, 84, 85, 86, 87, 88, 89, 90, 91, 92, 93, 94, 95, 96, 97, 98, 99, 100, 101, 144, 145, 146, 147, 148, 149, 150, 151, 152, 153, 154, 155, 156, 157, 158, 159, 160, 161, 162, 163, 164, 165, 166, 167, 168, or 169; and/or a light chain comprising the amino acid sequence of SEQ ID NO: 102, 103, 104, 105, 106, 107, 108, 109, 110, 111, 112, 113, 114, or 115. In certain embodiments, the amino acid sequence of the heavy chain consists of the amino acid sequence of SEQ ID NO: 76, 77, 78, 79, 80, 81, 82, 83, 84, 85, 86, 87, 88, 89, 90, 91, 92, 93, 94, 95, 96, 97, 98, 99, 100, 101, 144, 145, 146, 147, 148, 149, 150, 151, 152, 153, 154, 155, 156, 157, 158, 159, 160, 161, 162, 163, 164, 165, 166, 167, 168, or 169; and/or the amino acid sequence of the light chain consists of the amino acid sequence of SEQ ID NO: 102, 103, 104, 105, 106, 107, 108, 109, 110, 111, 112, 113, 114, or 115. In certain embodiments, the heavy chain and light chain comprise the amino acid sequences of SEQ ID NOs: 76 and 102; 79 and 103; 78 and 103; 82 and 103; 84 and 104; 83 and 104; 86 and 103; 85 and 103; 81 and 103; 80 and 103; 87 and 105; 77 and 102; 88 and 102; 77 and 106; 77 and 107; 77 and 108; 77 and 103; 89 and 102; 90 and 102; 91 and 102; 92 and 102; 93 and 102; 77 and 109; 94 and 102; 95 and 102; 96 and 102; 97 and 102; 98 and 102; 99 and 102; 100 and 110; 100 and 111; 100 and 112; 100 and 113; 100 and 114; 100 and 115; 101 and 110; 144 and 102; 147 and 103; 146 and 103; 150 and 103; 152 and 104; 151 and 104; 154 and 103; 153 and 103; 149 and 103; 148 and 103; 155 and 105; 145 and 102; 156 and 102; 145 and 106; 145 and 107; 145 and 108; 145 and 103; 157 and 102; 158 and 102; 159 and 102; 160 and 102; 161 and 102; 145 and 109; 162 and 102; 163 and 102; 164 and 102; 165 and 102; 166 and 102; 167 and 102; 168 and 110; 168 and 111; 168 and 112; 168 and 113; 168 and 114; 168 and 115; or 169 and 110, respectively. In certain embodiments, the amino acid sequences of the heavy chain and the light chain consist of the amino acid sequences of SEQ ID NOs: 76 and 102; 79 and 103; 78 and 103; 82 and 103; 84 and 104; 83 and 104; 86 and 103; 85 and 103; 81 and 103; 80 and 103; 87 and 105; 77 and 102; 88 and 102; 77 and 106; 77 and 107; 77 and 108; 77 and 103; 89 and 102; 90 and 102; 91 and 102; 92 and 102; 93 and 102; 77 and 109; 94 and 102; 95 and 102; 96 and 102; 97 and 102; 98 and 102; 99 and 102; 100 and 110; 100 and 111; 100 and 112; 100 and 113; 100 and 114; 100 and 115; 101 and 110; 144 and 102; 147 and 103; 146 and 103; 150 and 103; 152 and 104; 151 and 104; 154 and 103; 153 and 103; 149 and 103; 148 and 103; 155 and 105; 145 and 102; 156 and 102; 145 and 106; 145 and 107; 145 and 108; 145 and 103; 157 and 102; 158 and 102; 159 and 102; 160 and 102; 161 and 102; 145 and 109; 162 and 102; 163 and 102; 164 and 102; 165 and 102; 166 and 102; 167 and 102; 168 and 110; 168 and 111; 168 and 112; 168 and 113; 168 and 114; 168 and 115; or 169 and 110, respectively. In certain embodiments, the X in any one of SEQ ID NOs: 76-101 or 144-169 is glutamine. In certain embodiments, the X in any one of SEQ ID NOs: 76-101 or 144-169 is pyroglutamate.

In another aspect, the instant disclosure provides an isolated antibody that specifically binds to human CD96, wherein the antibody binds to the same epitope of human CD96 as an antibody disclosed herein.

In another aspect, the instant disclosure provides an isolated antibody that specifically binds to human CD96, wherein the antibody competes for binding to human CD96 with an antibody disclosed herein.

In certain embodiments, the antibody is a human antibody. In certain embodiments, the antibody is a multispecific antibody. In certain embodiments, the antibody is conjugated to a cytotoxic agent, cytostatic agent, toxin, radionuclide, or detectable label. In certain embodiments, the antibody is conjugated to a second antibody.

In another aspect, the instant disclosure provides an isolated polynucleotide encoding a VH and/or a VL of an antibody disclosed herein. In another aspect, the instant disclosure provides a vector comprising the polynucleotide. In another aspect, the instant disclosure provides a recombinant host cell comprising the polynucleotide or the vector. In another aspect, the instant disclosure provides a method of producing an antibody that specifically binds to human CD96, the method comprising culturing the host cell under suitable conditions so that the polynucleotide is expressed and the antibody is produced.

In another aspect, the instant disclosure provides a pharmaceutical composition comprising an antibody, a polynucleotide, a vector, or a host cell disclosed herein; and a pharmaceutically acceptable carrier or excipient.

In another aspect, the instant disclosure provides a method of increasing an immune response in a subject, the method comprising administering to the subject an effective amount of an antibody, a polynucleotide, a vector, a host cell, or a pharmaceutical composition disclosed herein.

In another aspect, the instant disclosure provides a method of treating cancer in a subject, the method comprising administering to the subject an effective amount of an antibody, a polynucleotide, a vector, a host cell, or a pharmaceutical composition disclosed herein.

In another aspect, the instant disclosure provides a method of treating an infectious disease in a subject, the method comprising administering to the subject an antibody, a polynucleotide, a vector a host cell, or a pharmaceutical composition disclosed herein

In certain embodiments of the foregoing methods, the antibody, polynucleotide, vector host cell, or pharmaceutical composition is administered, systemically, intravenously, subcutaneously, intratumorally, or is delivered to a tumor draining lymph node.

In certain embodiments of the foregoing methods, the methods further comprise administering an additional therapeutic agent to the subject. In certain embodiments, the additional therapeutic agent is a chemotherapeutic agent. In certain embodiments, the additional therapeutic agent is a checkpoint targeting agent. In certain embodiments, the checkpoint targeting agent is selected from the group consisting of an antagonist anti-PD-1 antibody, an antagonist anti-PD-LI antibody, an antagonist anti-PD-L2 antibody, an antagonist anti-CTLA-4 antibody, an antagonist anti-TIM-3 antibody, an antagonist anti-LAG-3 antibody, an antagonist anti-VISTA antibody, an antagonist anti-TIGIT antibody, an antagonist anti-CEACAMI antibody, an antagonist anti-CD96 antibody, an agonist anti-GITR antibody, and an agonist anti-OX₄₀ antibody. In certain embodiments, the additional therapeutic agent is an anti-PD-1 antibody, optionally wherein the anti-PD-1 antibody is pembrolizumab or nivolumab. In certain embodiments, the additional therapeutic agent is an inhibitor of indoleamine-2,3-dioxygenase (IDO). In certain embodiments, the inhibitor is selected from the group consisting of epacadostat, F001287, indoximod, and NLG919. In certain embodiments, the additional therapeutic agent is a vaccine. In certain embodiments, the vaccine comprises a heat shock protein peptide complex (HSPPC) comprising a heat shock protein complexed with an antigenic peptide. In certain embodiments, the heat shock protein is hsc70 and is complexed with a tumor-associated antigenic peptide. In certain embodiments, the heat shock protein is gp96 protein and is complexed with a tumor-associated antigenic peptide, wherein the HSPPC is derived from a tumor obtained from a subject.

### 4. BRIEF DESCRIPTION OF THE DRAWINGS

FIGS. 1A and 1B are graphs showing the binding of the anti-CD96 antibodies BA072 or BA101. or an IgG1 isotype control antibody, to Jurkat cells engineered to express high levels of cell surface human isoform 2 of CD96. The levels of Jurkat cell binding of BA072 (FIG. 1A) or BA101 (FIG. 1B), as assessed by median fluorescence intensity (MFI), in each case in comparison with Jurkat cell binding of an IgG1 isotype control antibody, are plotted against the concentrations of the respective antibody incubated with the cells.

FIGS. 2A and 2B are graphs showing the binding of the anti-CD96 antibodies BA072 or BA101, or an IgG1 isotype control antibody, to CHO cells engineered to express high levels of cell surface isoform 1 of human CD96. The levels of binding of BA072 (FIG. 2A) or BA101 (FIG. 2B), as assessed by median fluorescence intensity (MFI), in each case in comparison with CHO cell binding of an IgG1 isotype control antibody, are plotted against the concentrations of the respective antibody incubated with the cells.

FIGS. 3A and 3B are graphs showing the binding of the anti-CD96 antibodies BA072 or BA101, or an IgG1 isotype control antibody, to CHO cells engineered to express high levels of cell surface isoform 2 of human CD96. The levels of binding of BA072 (FIG. 3A) or BA101 (FIG. 3B), as assessed by median fluorescence intensity (MFI), in each case in comparison with CHO cell binding of an IgG1 isotype control antibody, are plotted against the concentrations of the respective antibody incubated with the cells.

FIGS. 4A and 4B are graphs showing the binding of the anti-CD96 antibodies BA072 or BA101, or an IgG1 isotype control antibody, to CHO cells engineered to express high levels of cell surface isoform 2 of cynomolgus monkey CD96. The levels of binding of BA072 (FIG. 4A) or BA101 (FIG. 4B), as assessed by median fluorescence intensity (MFI), in each case in comparison with CHO cell binding of an IgG1 isotype control antibody, are plotted against the concentrations of the respective antibody incubated with the cells.

FIGS. 5A and 5B are graphs showing the binding of the anti-CD96 antibodies BA072 or BA101, or an IgG1 isotype control antibody, to activated primary human T cells expressing cell surface CD96. The levels of binding of BA072 (FIG. 5A) or BA101 (FIG. 5B), as assessed by median fluorescence intensity (MFI), in each case in comparison with activated primary human T cell binding of an IgG1 isotype control antibody, are plotted against the concentrations of the respective antibody incubated with the cells.

FIGS. 6A, 6B, and 6C are a series of graphs showing the binding of the anti-CD96 antibodies BA072, BA083, or BA084, or an IgG1 isotype control antibody, to activated primary human T cells expressing cell surface CD96. The levels of binding of BA072 (FIG. 6A), BA083 (FIG. 6B), or BA084 (FIG. 6C), as assessed by median fluorescence intensity (MFI), in each case in comparison with activated primary human T cell binding of an IgG1 isotype control antibody, are plotted against the concentrations of the respective antibody incubated with the cells.

FIGS. 7A-7F are a series of graphs showing the binding of the anti-CD96 antibodies BA101, BA102, BA103, BA104, BA105, or BA106, or an IgG1 isotype control antibody, to activated primary human T cells expressing cell surface CD96. The levels of binding of BA101 (FIG. 7A), BA102 (FIG. 7B), BA103 (FIG. 7C), BA104 (FIG. 7D), BA105 (FIG. 7E), or BA106 (FIG. 7F), as assessed by median fluorescence intensity (MFI), in each case in comparison with activated primary human T cell binding of an IgG1 isotype control antibody, are plotted against the concentrations of the respective antibody incubated with the cells.

FIGS. 8A-8M are a series of graphs showing the binding of affinity-matured anti-CD96 antibodies, BA074, BA073, BA079, BA078, BA081, BA080, BA077, BA076, BA082, or BA075, parental antibodies BA072 or BA101, germlined antibody BA083, or an IgG1 isotype control antibody, to NY-ESO-1 transfected CD8′ T cells expressing cell surface CD96. The levels of binding of BA072 (FIG. 8A), BA083 (FIG. 8B), BA074 (FIG. 8C), BA073 (FIG. 8D), BA079 (FIG. 8E), BA078 (FIG. 8F), BA081 (FIG. 8G), BA080 (FIG. 8H), BA077 (FIG. 8I), BA076 (FIG. 8J), BA082 (FIG. 8K), BA075 (FIG. 8L), or BA101 (FIG. 8M), as assessed by median fluorescence intensity (MFI), in each case in comparison with NY-ESO-1 transfected CD8⁺ T cell binding of an IgG1 isotype control antibody, are plotted against the concentrations of the respective antibody incubated with the cells.

FIGS. 9A and 9B are graphs showing the binding of the anti-CD96 antibodies BA072 or BA101, or an IgG1 isotype control antibody, to activated cynomolgus monkey primary T cells expressing cell surface cynomolgus monkey CD96. The levels of binding of BA072 (FIG. 9A) or BA101 (FIG. 9B), as assessed by median fluorescence intensity (MFI), in each case in comparison with activated primary cynomolgus T cell binding of an IgG1 isotype control antibody, are plotted against the concentrations of the respective antibody incubated with the cells.

FIGS. 10A and 10B are graphs showing the blockade of PVR-Fc binding to CHO cells, engineered to express high levels of cell surface isoform 2 of human CD96, by the anti-CD96 antibodies BA072 (FIG. 10A) or BA101 (FIG. 10B). The levels of binding of PVR-Fc, as assessed by median fluorescence intensity (MFI), in each case in comparison with blockade by an IgG1 isotype control antibody, are plotted as % maximal response against the concentrations of the respective antibody incubated with the cells.

FIGS. 11A and 11B are graphs showing the blockade PVR-His binding to CHO cells, engineered to express high levels of cell surface isoform 2 of human CD96, by the anti-CD96 antibodies BA072 (FIG. 11A) or BA101 (FIG. 11B). The levels of binding of PVR-His, as assessed by median fluorescence intensity (MFI), in each case in comparison with blockade by an IgG1 isotype control antibody, are plotted as % maximal response against the concentrations of the respective antibody incubated with the cells.

FIGS. 12A-12C are a series of graphs showing the blockade of PVR-Fc binding to CHO cells, engineered to express high levels of cell surface isoform 2 of human CD96, by the anti-CD96 antibodies BA072 (FIG. 12A), BA083 (FIG. 12B), or BA084 (FIG. 12C). The levels of binding of PVR-Fc, as assessed by median fluorescence intensity (MFI), in each case in comparison with blockade by an IgG1 isotype control antibody, are plotted as % maximal response against the concentrations of the respective antibody incubated with the cells.

FIGS. 13A-13L are a series of graphs showing the blockade of human PVR-Fc binding to CHO cells, engineered to express high levels of cell surface isoform 2 of human CD96, by the anti-CD96 antibodies BA072 (FIG. 13A), BA083 (FIG. 13B), BA085 (FIG. 13C), BA086 (FIG. 13D), BA087 (FIG. 13E), BA089 (FIG. 13F), BA090 (FIG. 13G), BA088 (FIG. 13H), BA091 (FIG. 13I), BA092 (FIG. 13J), BA093 (FIG. 13K), or BA094 (FIG. 13L). The levels of binding of PVR-Fc, as assessed by median fluorescence intensity (MFI), in each case in comparison with blockade by an IgG1 isotype control antibody, are plotted as % maximal response against the concentrations of the respective antibody incubated with the cells.

FIGS. 14A-14L are a series of graphs showing the blockade of human PVR-Fc binding to CHO cells, engineered to express high levels of cell surface isoform 1 of human CD96, by the anti-CD96 antibodies BA073 (FIG. 14A), BA074 (FIG. 14B), BA078 (FIG. 14C), BA079 (FIG. 14D), BA080 (FIG. 14E), BA081 (FIG. 14F), BA076 (FIG. 14G), BA077 (FIG. 14H), BA082 (FIG. 14I), BA075 (FIG. 14J), BA083 (FIG. 14K), or BA072 (FIG. 14L). The levels of binding of PVR-Fc, as assessed by median fluorescence intensity (MFI), in each case in comparison with blockade by an IgG1 isotype control antibody, are plotted as % maximal response against the concentrations of the respective antibody incubated with the cells.

FIGS. 15A-15L are a series of graphs showing the blockade of human PVR-Fc binding to CHO cells, engineered to express high levels of cell surface isoform 2 of human CD96, by the anti-CD96 antibodies BA073 (FIG. 15A), BA074 (FIG. 15B), BA078 (FIG. 15C). BA079 (FIG. 15D), BA080 (FIG. 15E), BA081 (FIG. 15F), BA076 (FIG. 15G), BA077 (FIG. 15H), BA082 (FIG. 151), BA075 (FIG. 15J), BA083 (FIG. 15K), or BA072 (FIG. 15L). The levels of binding of PVR-Fc, as assessed by median fluorescence intensity (MFI), in each case in comparison with blockade by an IgG1 isotype control antibody, are plotted as % maximal response against the concentrations of the respective antibody incubated with the cells.

FIGS. 16A-16F are a series of graphs showing the blockade of PVR-Fc binding to CHO cells, engineered to express high levels of cell surface human isoform 2 of CD96, by the anti-CD96 antibodies BA101 (FIG. 16A), BA102 (FIG. 16B), BA103 (FIG. 16C), BA104 (FIG. 16D), BA105 (FIG. 16E), or BA106 (FIG. 16F). The levels of binding of PVR-Fc, as assessed by median fluorescence intensity (MFI), in each case in comparison with blockade by an IgG1 isotype control antibody, are plotted as % maximal response against the concentrations of the respective antibody incubated with the cells.

FIGS. 17A and 17B are graphs showing the blockade of PVR-Fc binding to CHO cells, engineered to express high levels of cell surface human isoform 2 of CD96, by the anti-CD96 antibodies BA101 (FIG. 17A) or BA107 (FIG. 17B). The levels of binding of PVR-Fc, as assessed by median fluorescence intensity (MFI), in each case in comparison with blockade by an IgG1 isotype control antibody, are plotted as % maximal response against the concentrations of the respective antibody incubated with the cells.

FIGS. 18A-18C are a series of graphs showing the blockade of PVR-Fc binding to CHO cells, engineered to express high levels of cell surface isoform 2 of cynomolgus monkey CD96, by the anti-CD96 antibodies BA072 (FIG. 18A), BA083 (FIG. 18B), or BA084 (FIG. 18C). The levels of binding of PVR-Fc, as assessed by median fluorescence intensity (MFI), in each case in comparison with blockade by an IgG1 isotype control antibody, are plotted as % maximal response against the concentrations of the respective antibody incubated with the cells.

FIGS. 19A-19L are a series of graphs showing the blockade of human PVR-Fc binding to CHO cells, engineered to express high levels of cell surface isoform 2 of cynomolgus monkey CD96, by the anti-CD96 antibodies BA072 (FIG. 19A), BA083 (FIG. 19B), BA085 (FIG. 19C), BA086 (FIG. 19D), BA088 (FIG. 19E), BA087 (FIG. 19F), BA089 (FIG. 19G), BA090 (FIG. 19H), BA091 (FIG. 19I), BA092 (FIG. 19J), BA093 (FIG. 19K), or BA094 (FIG. 19L). The levels of binding of PVR-Fc, as assessed by median fluorescence intensity (MFI), in each case in comparison with blockade by an IgG1 isotype control antibody, are plotted as % maximal response against the concentrations of the respective antibody incubated with the cells.

FIGS. 20A-20L are a series of graphs showing the blockade of human PVR-Fc binding to CHO cells, engineered to express high levels of cell surface isoform 1 of cynomolgus CD96, by the anti-CD96 antibodies BA073 (FIG. 20A), BA074 (FIG. 20B), BA078 (FIG. 20C), BA079 (FIG. 20D), BA080 (FIG. 20E), BA081 (FIG. 20F), BA076 (FIG. 20G), BA077 (FIG. 20H), BA082 (FIG. 20I), BA075 (FIG. 20J), BA083 (FIG. 20K), or BA072 (FIG. 20L). The levels of binding of PVR-Fc, as assessed by median fluorescence intensity (MFI), in each case in comparison with blockade by an IgG1 isotype control antibody, are plotted as % maximal response against the concentrations of the respective antibody incubated with the cells.

FIGS. 21A-21L are a series of graphs showing the blockade of human PVR-Fc binding to CHO cells, engineered to express high levels of cell surface isoform 2 of cynomolgus CD96, by the anti-CD96 antibodies BA073 (FIG. 21A), BA074 (FIG. 21B), BA078 (FIG. 21C), BA079 (FIG. 21D), BA080 (FIG. 21E), BA081 (FIG. 21F), BA076 (FIG. 21G), BA077 (FIG. 21H), BA082 (FIG. 21I), BA075 (FIG. 21J), BA083 (FIG. 21K), or BA072 (FIG. 21L). The levels of binding of PVR-Fc, as assessed by median fluorescence intensity (MFI), in each case in comparison with blockade by an IgG1 isotype control antibody, are plotted as % maximal response against the concentrations of the respective antibody incubated with the cells.

FIGS. 22A and 22B are graphs showing the conjugate formation of CHO cells, engineered to express high levels of isoform 2 of human CD96 or PVR, in the presence of the anti-CD96 antibodies BA072 (FIG. 22A) or BA101 (FIG. 22B), or an IgG1 isotype control antibody. The percent of conjugates formed, in each case in comparison to IgG1 isotype control, are plotted against the concentrations of the respective antibody incubated with the cells. FIG. 22C are scatter plots showing conjugate formation in quadrant Q2 in the presence of isotype control, and not in the presence of blocking antibody.

FIG. 23 is a graph showing the conjugate formation of CHO cells, engineered to express high levels of isoform 2 of human CD96 or PVR, in the presence of the anti-CD96 antibodies BA072, BA083, BA084, or an IgG1 isotype control antibody. The percent of conjugates formed, in each case in comparison to IgG1 isotype control, are plotted against the concentrations of the respective antibody incubated with the cells.

FIG. 24 is a graph showing the conjugate formation of CHO cells, engineered to express high levels of isoform 2 of human CD96 or PVR, in the presence of anti-CD96 antibodies BA101, BA102, BA103, BA104, BA105, or BA106. The percent of conjugates formed, in each case in comparison to IgG1 isotype control, are plotted against the concentrations of the respective antibody incubated with the cells.

FIGS. 25A-25H are a series of graphs showing that the anti-CD96 antibodies BA072 and BA101 promote IL-2 secretion by SEA-stimulated PBMCs in a dose-dependent manner, when administered with and without an anti-PD-1 antibody, in two different donors. FIGS. 25A-D represent a first experiment with a first donor, and FIGS. 25E-H represent a second experiment with a second donor.

FIGS. 26A-26F are a series of graphs showing that the affinity-matured BA073, BA078, BA080, and BA076 antibodies and the germlined antibody BA083 promote IL-2 secretion by SEA-stimulated PBMCs both with and without an anti-PD-1 antibody. FIGS. 26A and 26B represent one experiment without (FIG. 26A) and with (FIG. 26B) an anti-PD-1 antibody. FIGS. 26C and 26D represent a second experiment, with a different donor, without (FIG. 26C) and with (FIG. 26D) an anti-PD-1 antibody. FIGS. 26E and 26F represent a third experiment, with a different donor, without (FIG. 26E) and with (FIG. 26F) an anti-PD-1 antibody.

FIGS. 27A-27F are a series of graphs showing the ability of affinity-matured BA074, BA079, BA077, BA081, BA082, and BA075 antibodies and the parental BA072 antibody to promote IL-2 secretion by SEA-stimulated PBMCs. FIGS. 27A and 27B represent one experiment without (FIG. 27A) and with (FIG. 27B) an anti-PD-1 antibody. FIGS. 27C and 27D represent a second experiment, with a different donor, without (FIG. 27C) and with (FIG. 27D) an anti-PD-1 antibody. FIGS. 27E and 27F represent a third experiment, with a different donor, without (FIG. 27E) and with (FIG. 27F) an anti-PD-1 antibody.

FIGS. 28A and 28B are graphs showing the increase in NFAT-Luciferase (FIG. 28A) and NFKB-Luciferase (FIG. 28B) signaling on CD96-expressing Jurkat reporter cells in the presence of BA072 and PVR and anti-CD3 expressing CHO cells. The delta relative light units (RLU) between BA072 and isotype control is plotted against antibody concentration. FIGS. 28C-1 and 28C-2 are a series of histograms showing cell surface expression of CD96, CD226, PVR, and CD3.

FIGS. 29A and 29B are a series of graphs showing the increase in NFAT-Luciferase signaling on CD96-expressing Jurkat reporter cells, with (FIG. 29A) and without (FIG. 29B) CD226 surface expression, in the presence of BA072 and PVR and anti-CD3 expressing CHO cells. The delta relative light units (RLU) between BA072 and isotype control is plotted against antibody concentration.

FIGS. 30A-30C are a series of graph showing promotion of antibody-dependent cell-mediated cytotoxicity (ADCC) of CD96-expressing cells in the presence of primary NK cells as measured by induction of caspase 3/7 activation by BA072 IgG1 (FIG. 30A), Fc-enhanced BA072 (BA109) (FIG. 30B), or the Fc-silent variant of BA072 (BA108) (FIG. 30C), in each case in comparison to an isotype control. The % induced caspase 3/7 activation is plotted against time (h).

FIGS. 31A-31C are a series of graphs showing FcγRIIIA-mediated NFAT signaling from FcγRIIIA-expressing Jurkat reporter cells in the presence of anti-CD96 BA072 Fc variants, Fc-enhanced BA072 variant (BA109) (FIG. 31B), Fc-silent variant of BA072 (BA108) (FIG. 31C), or BA072 IgG1 (FIG. 31A), bound to CD96-expressing target cells (4:1 E:T ratio). The relative light units (RLU) is plotted against antibody concentration.

FIGS. 32A and 32B are graphs showing the extent of IL-2 secretion elicited by BA072 (FIG. 32A) and BA108 (an Fc silent variant of BA072: FIG. 32B) in T cell: APC co-culture assays using PBMCs from two human donors.

FIGS. 33A-33D are a series of graphs showing percent internalization of CD96 using CD96-expressing Jurkat cells in the presence of BA072 (FIG. 33A), BA101 (FIG. 33B), the reference antibody Reference A (FIG. 33C), or PVR-Fc (FIG. 33D).

FIGS. 34A-34D are a series of graphs showing the percent internalization of CD96 using CD96-expressing Jurkat cells in the presence of parental antibodies BA072 (FIG. 34A) or BA101 (FIG. 34D), or germline variants BA083 (FIG. 34B) or BA084 (FIG. 34C).

FIGS. 35A and 35B are graphs showing internalization of CD96 by CD96-expressing primary T cells in the presence of BA072 in donor 1 (FIG. 35A) and donor 2 (FIG. 35B).

FIGS. 36A and 36B are sensorgrams showing binding of BA072 Fab, BA101 Fab, and Reference A Fab to Fc-tagged full-length human CD96 (FIG. 36A) or Fc-tagged domain 1 of human CD96 (FIG. 36B).

FIGS. 37A-37D are a series of sensorgrams showing binding of BA072 Fab, BA101 Fab, and Reference A Fab to Fc-tagged full-length human CD96 (FIGS. 37A and 37B) or Fc-tagged domain 1 human CD96 (FIGS. 37C and 37D). FIGS. 37A and 37C represent experiments where initial association was with BA072. FIGS. 37B and 37D represent experiments where initial association was with BA101.

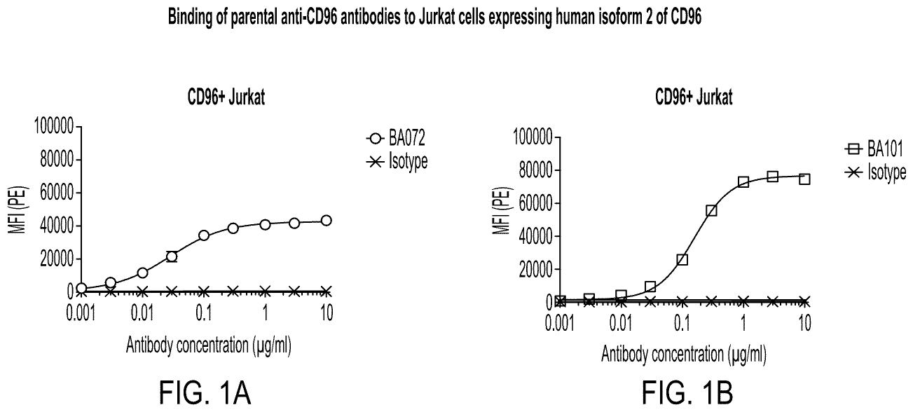

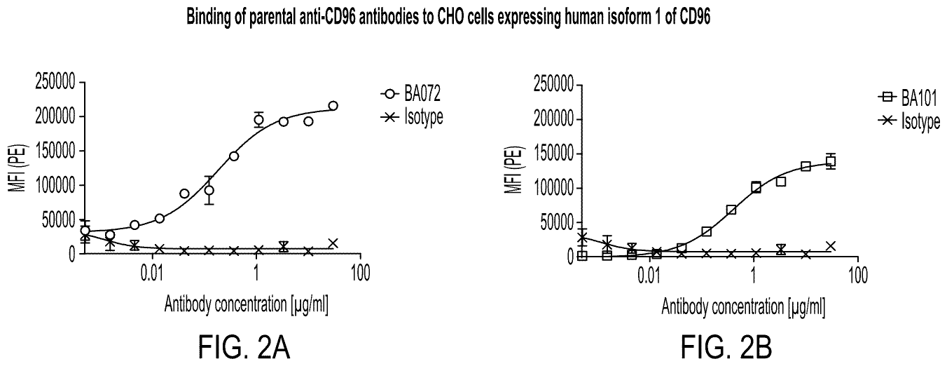

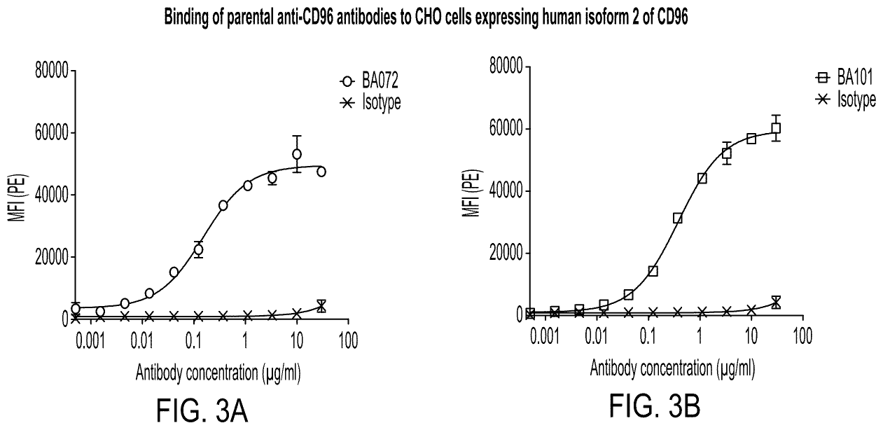

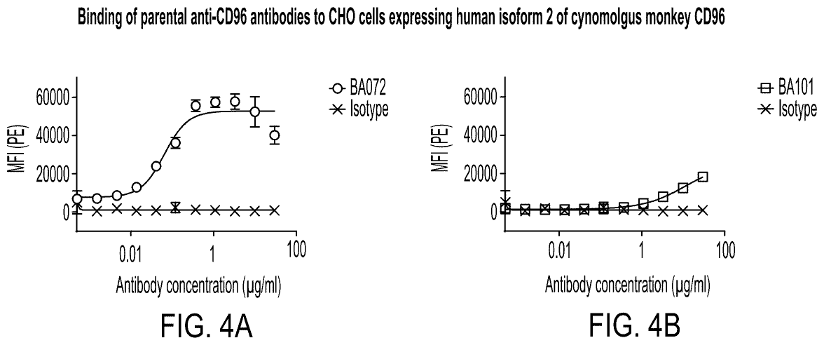

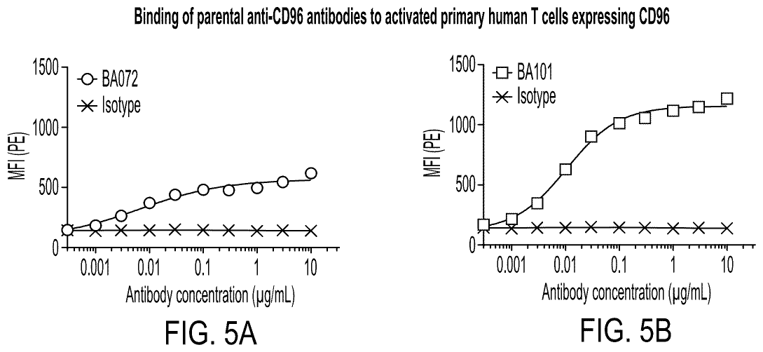

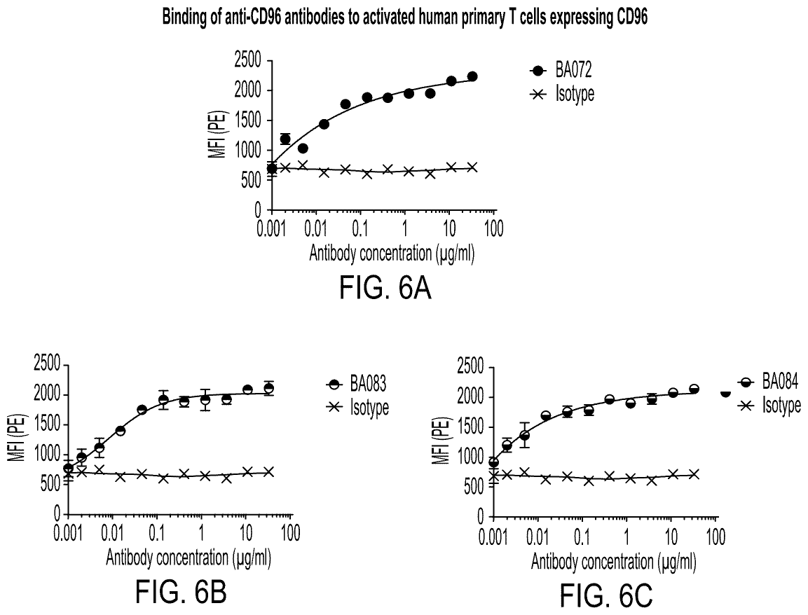

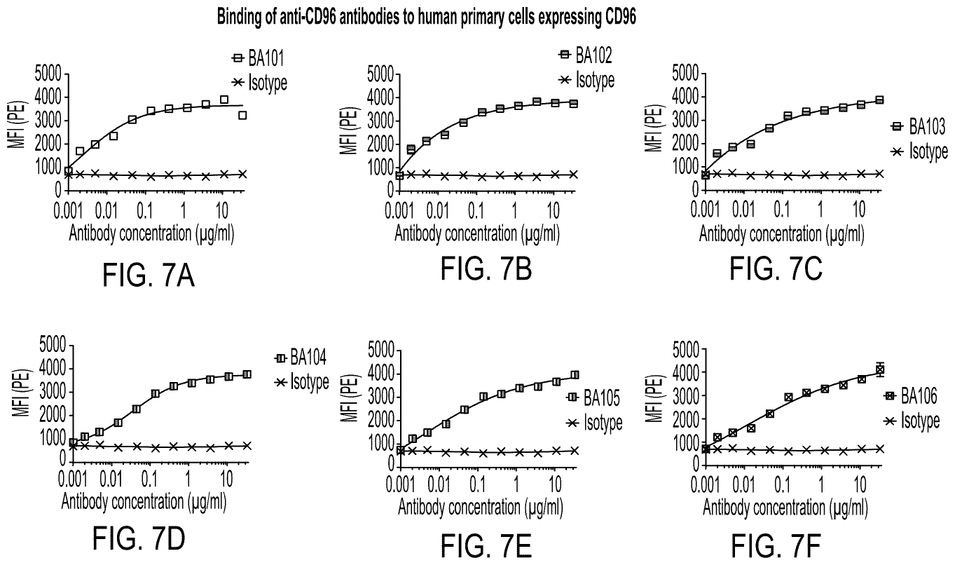

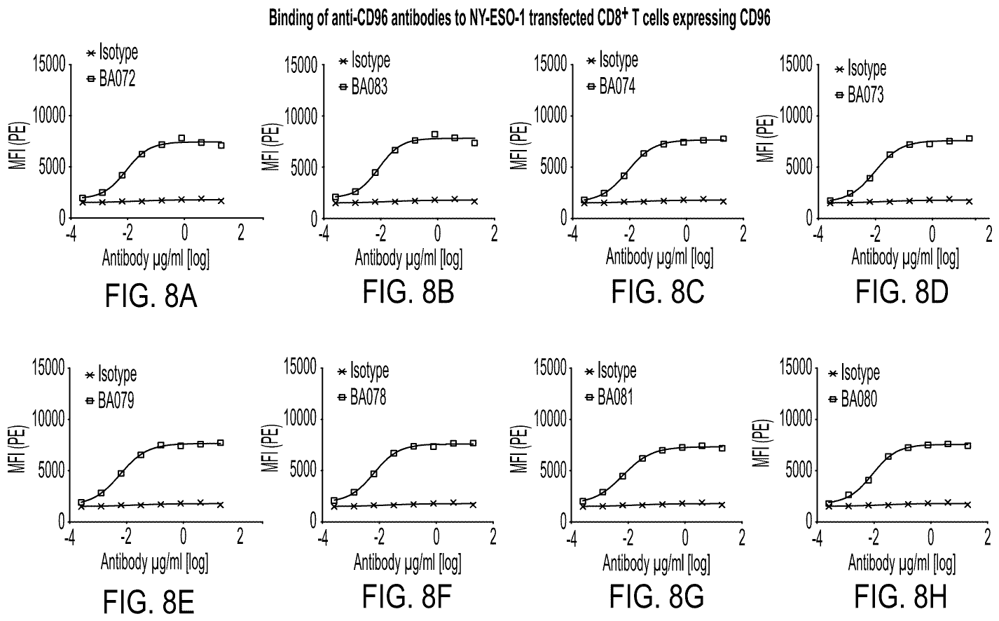

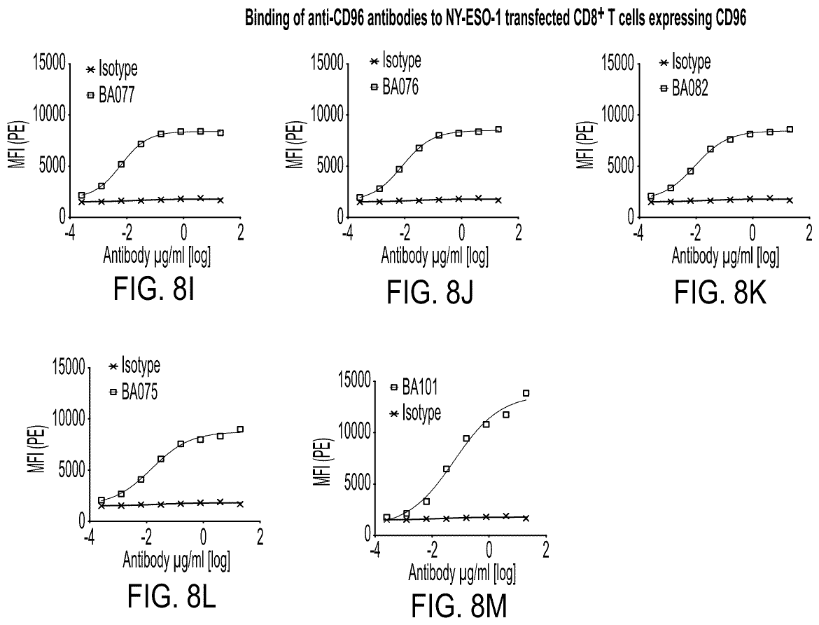

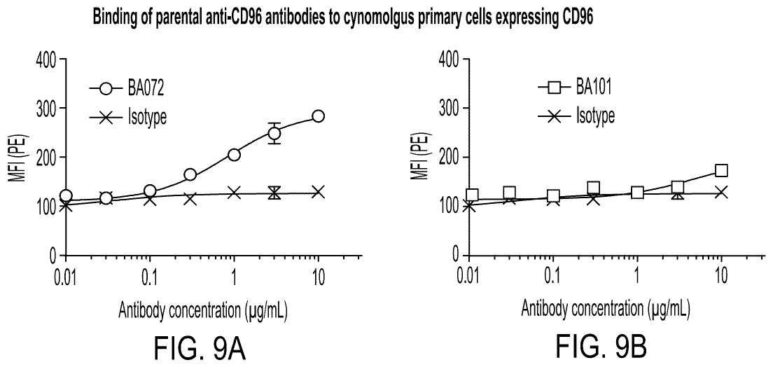

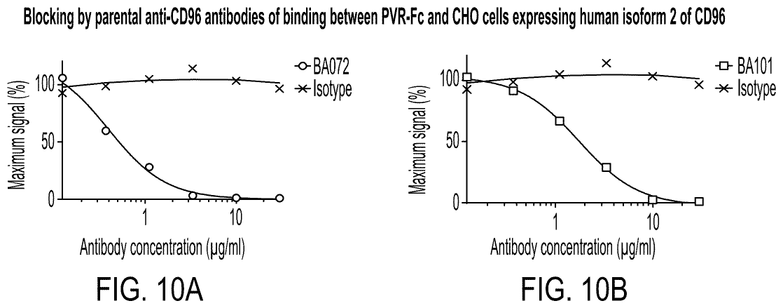

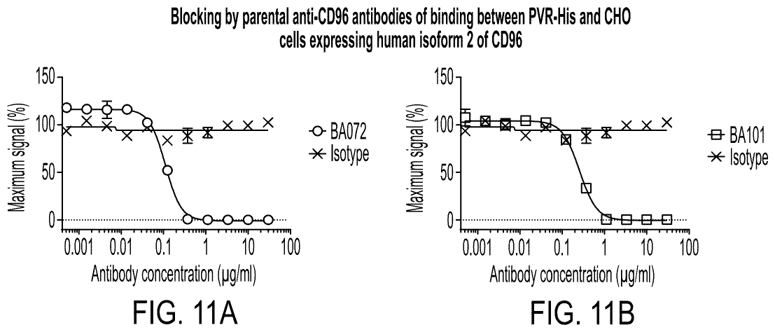

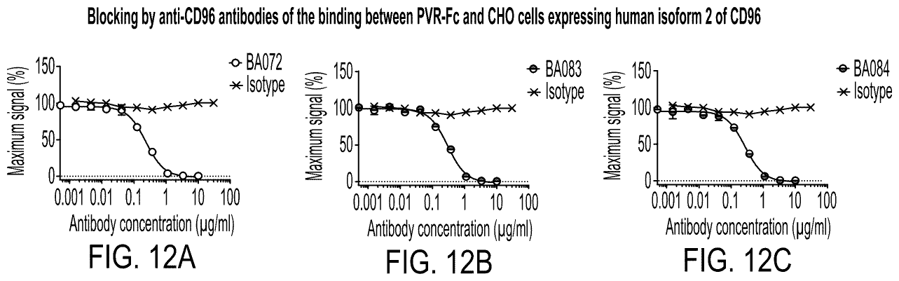

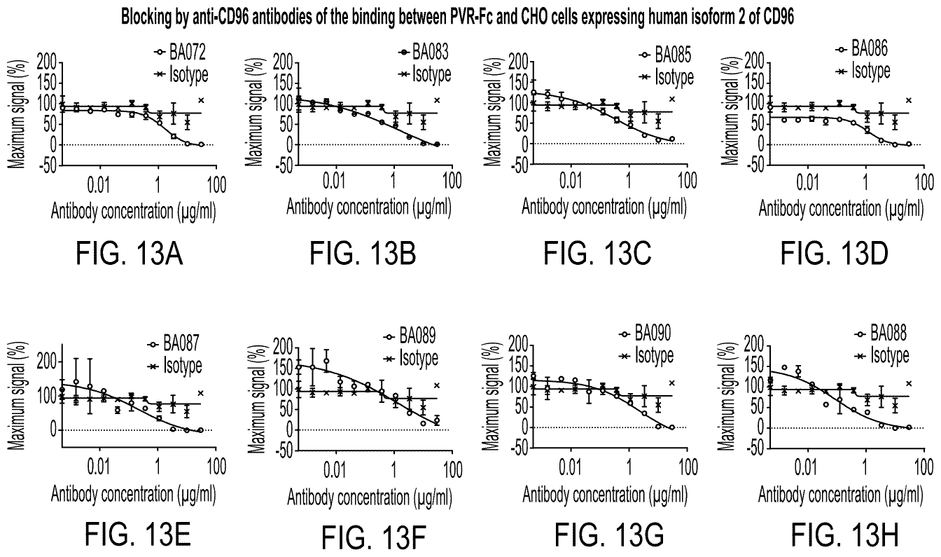

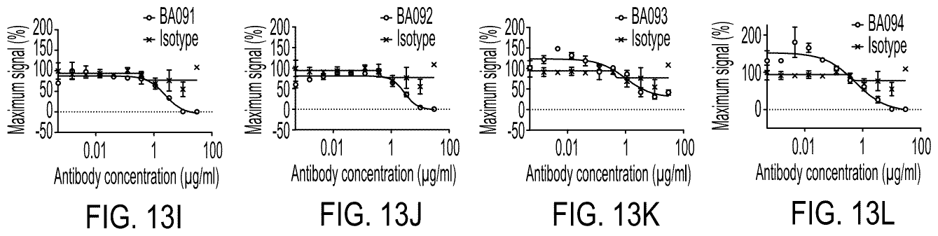

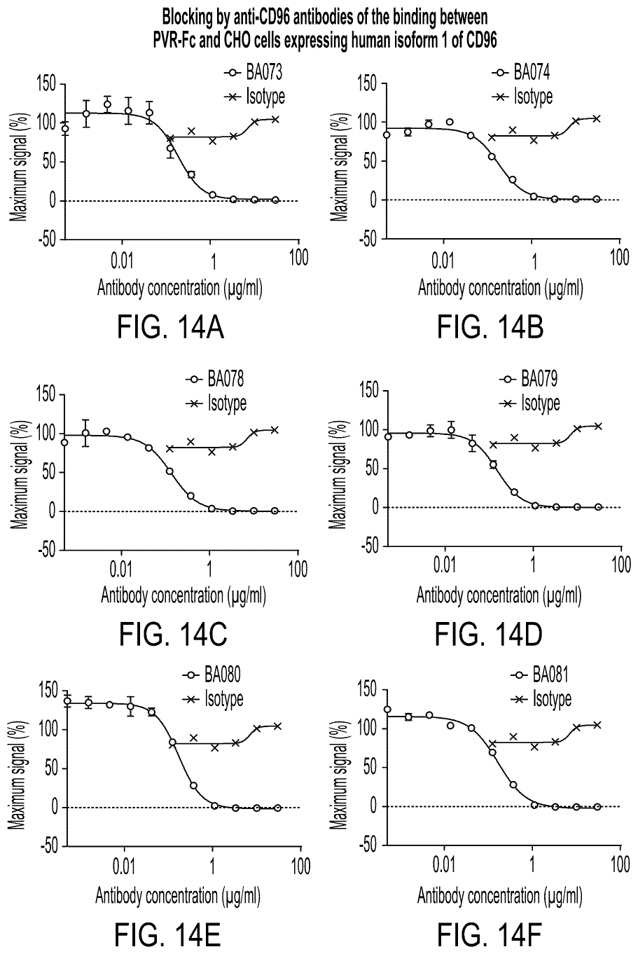

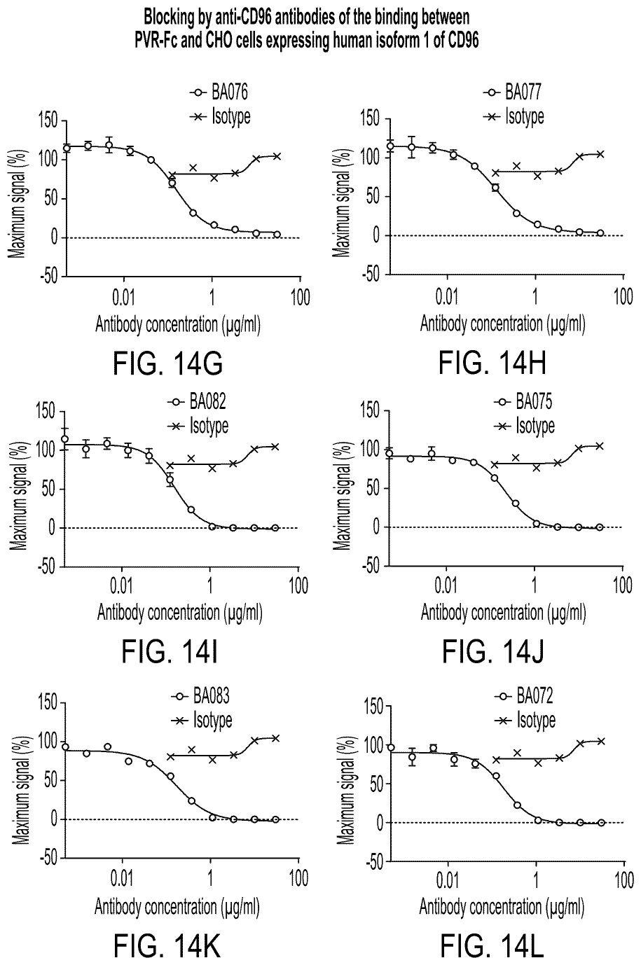

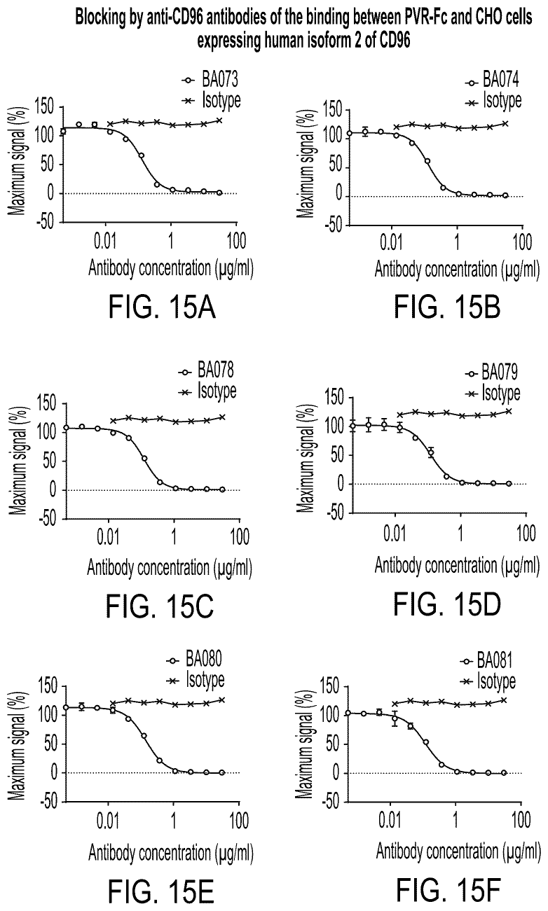

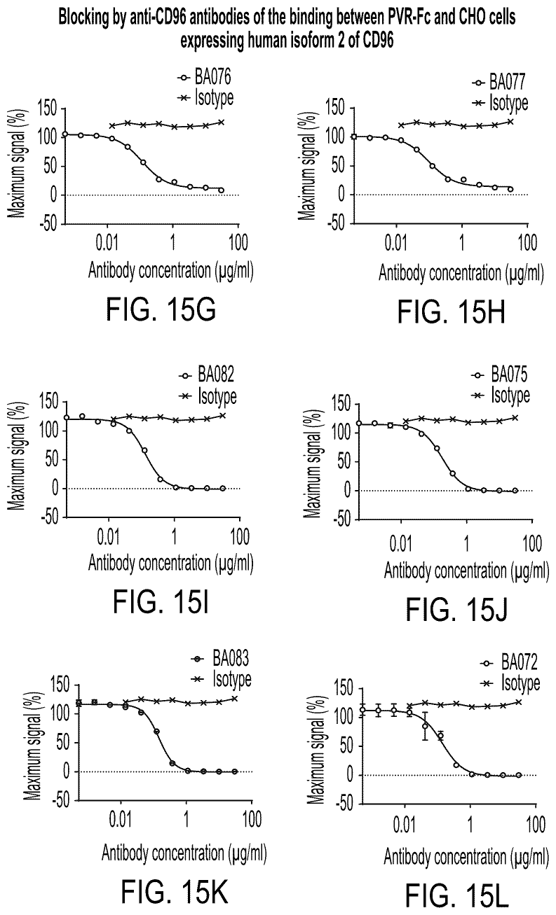

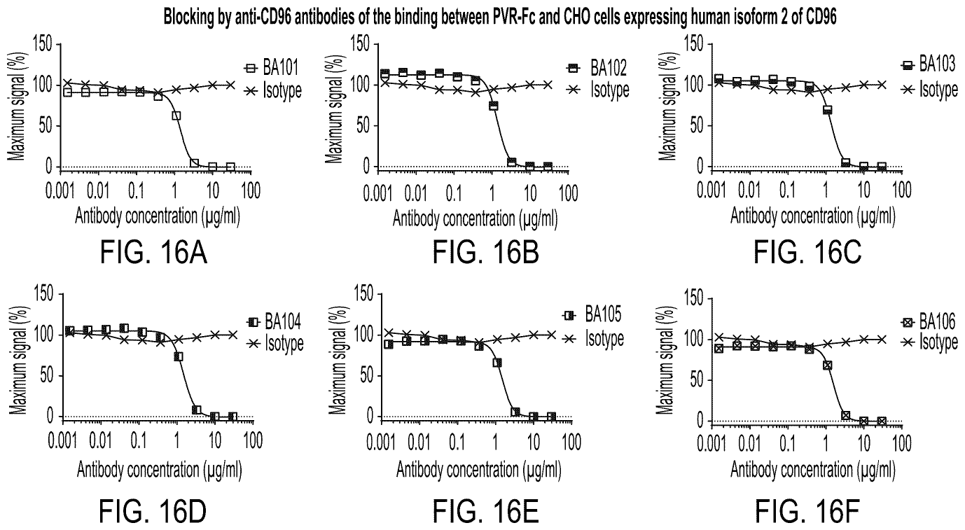

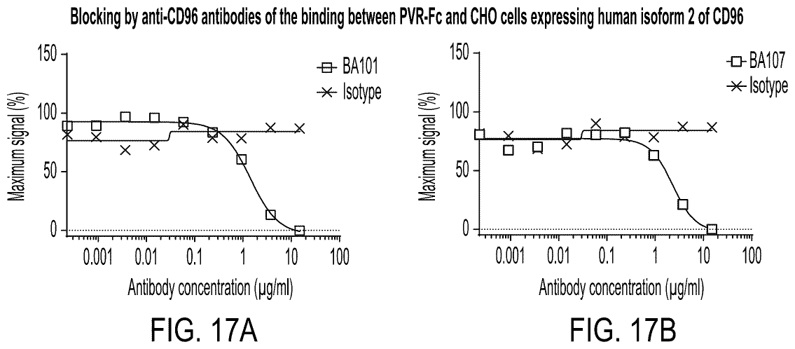

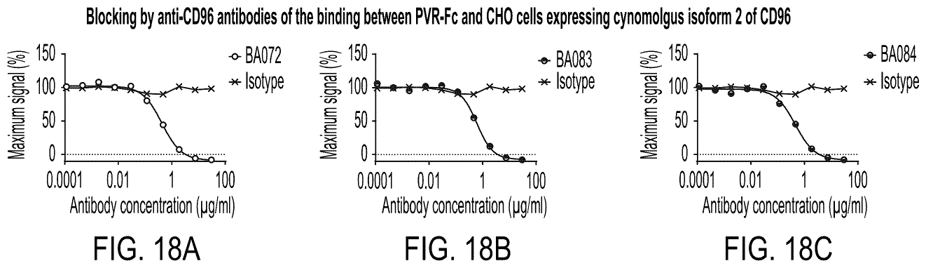

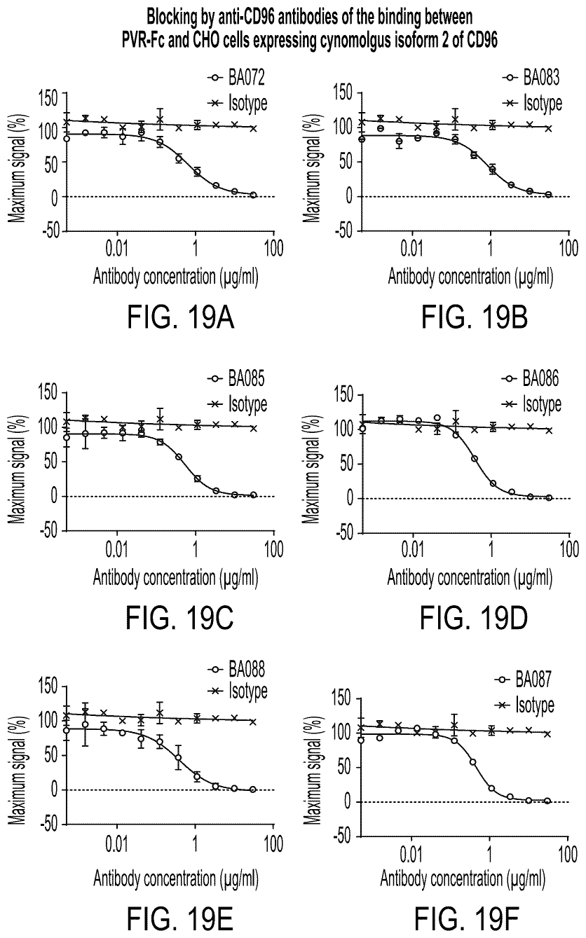

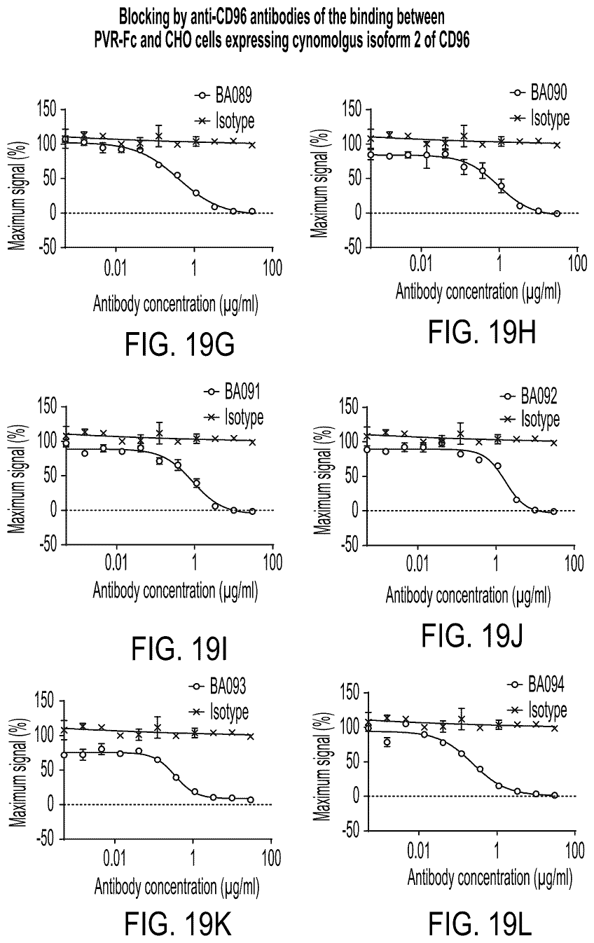

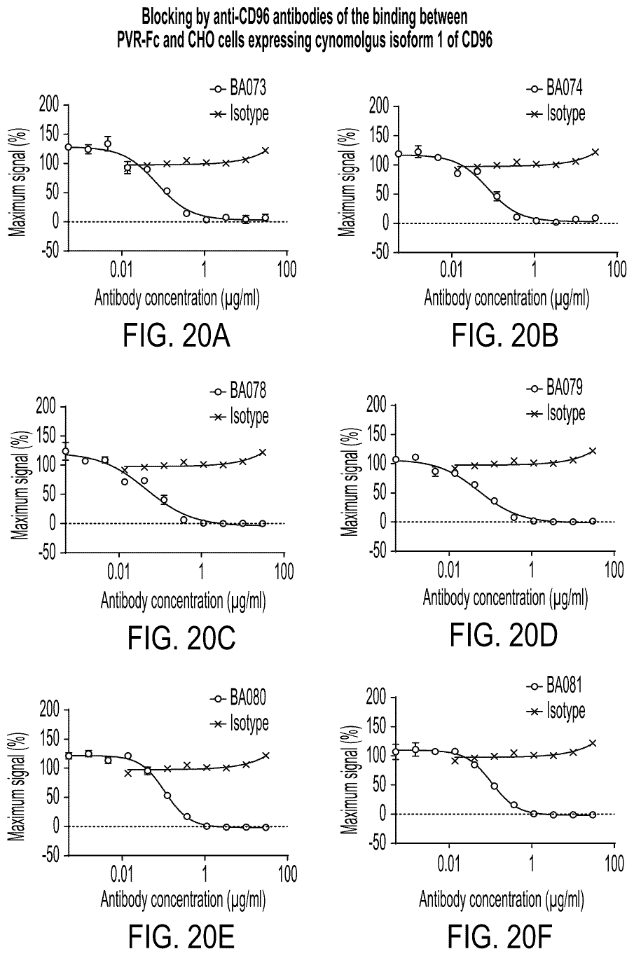

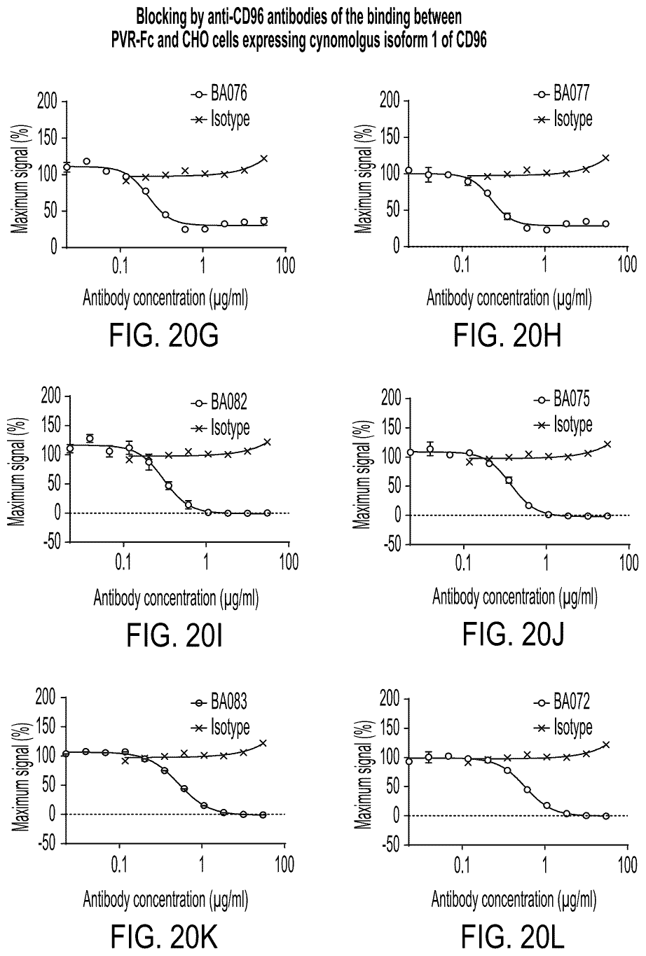

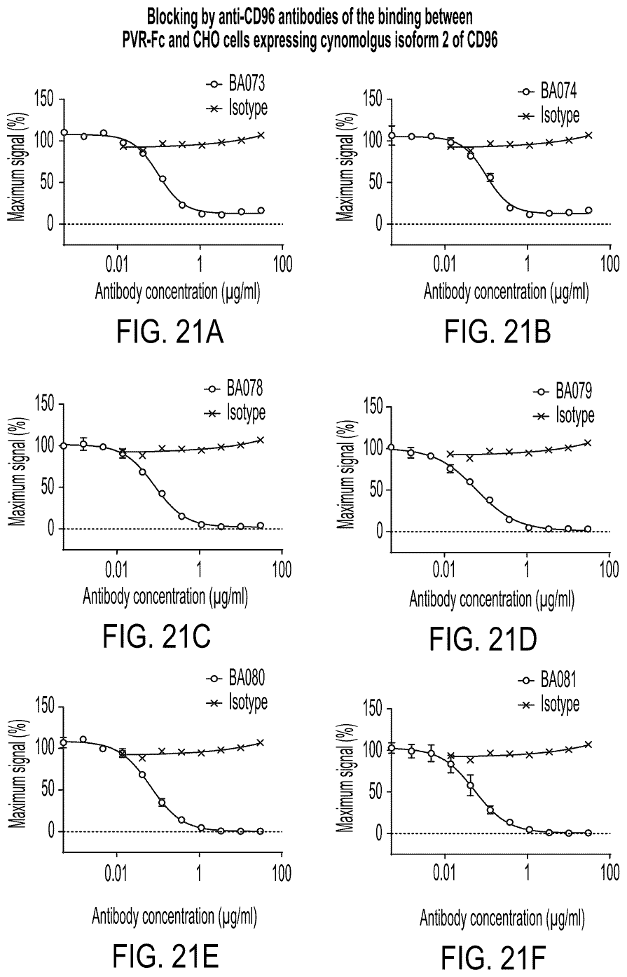

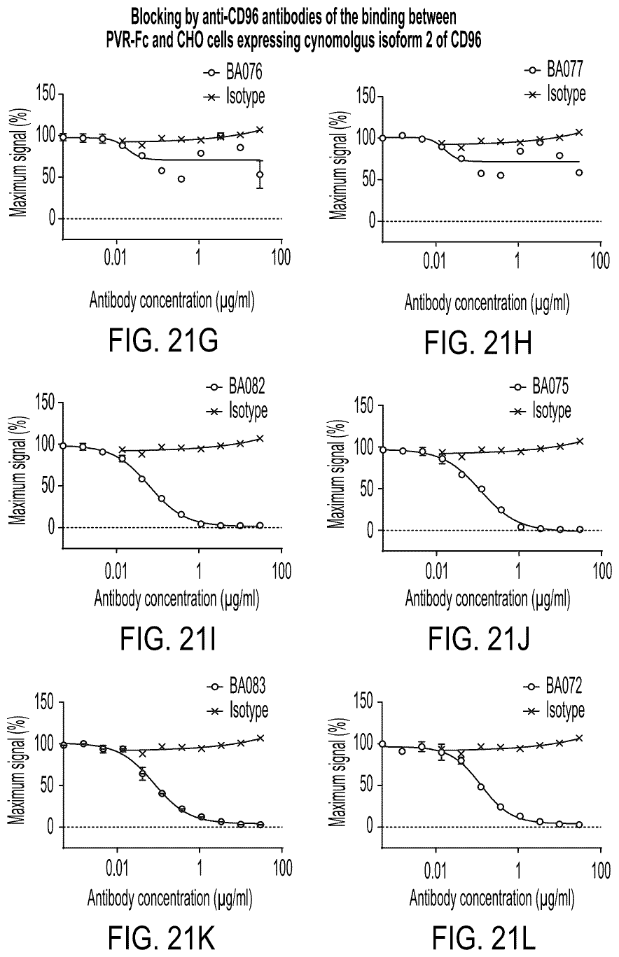

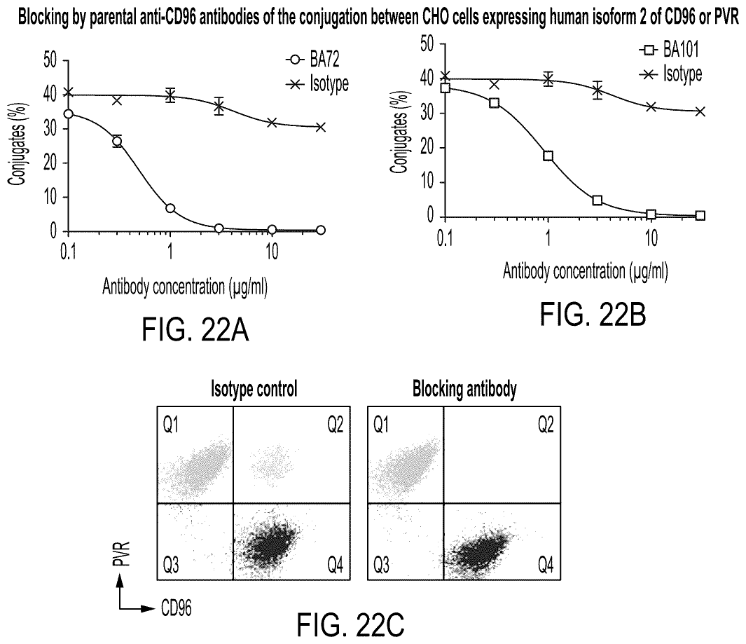

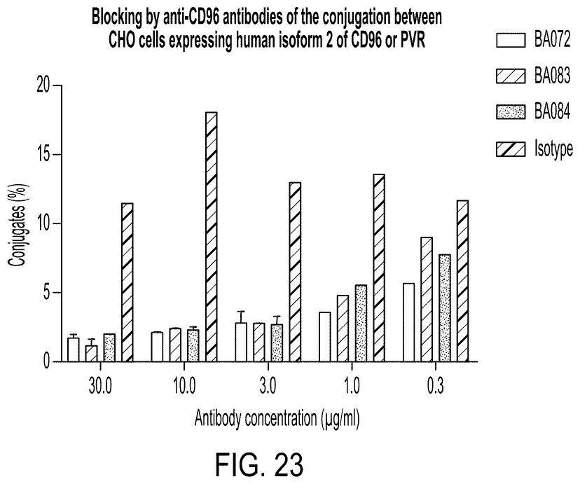

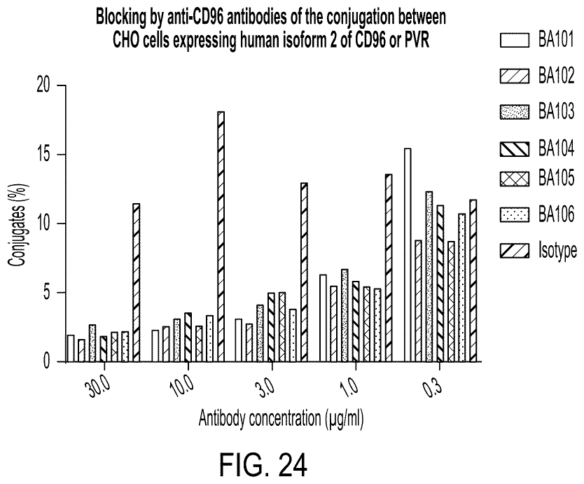

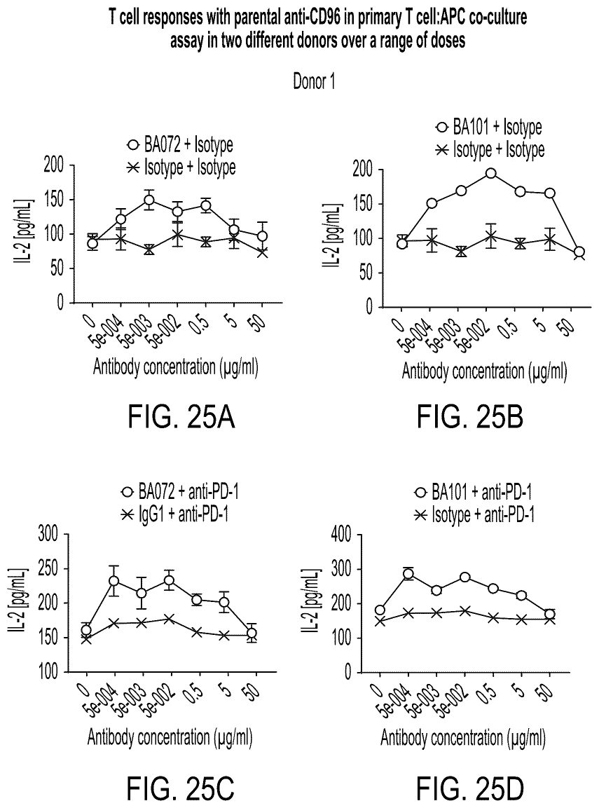

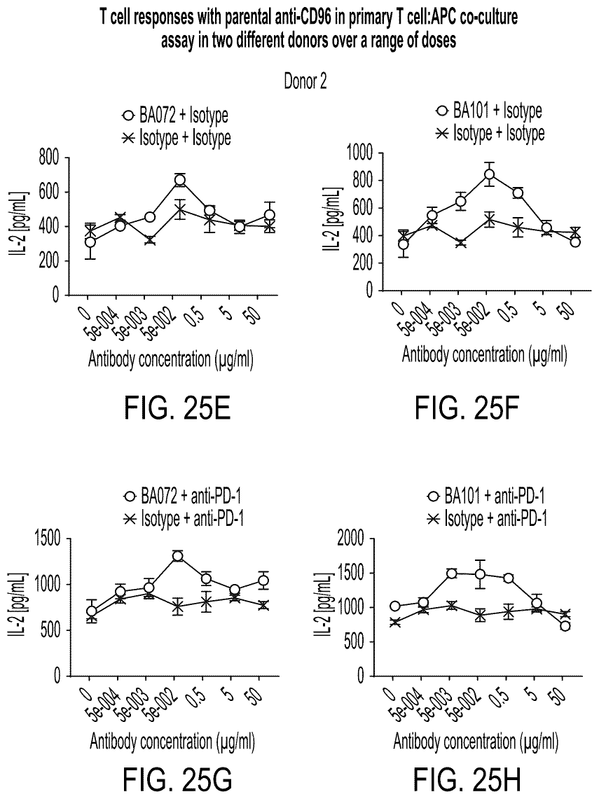

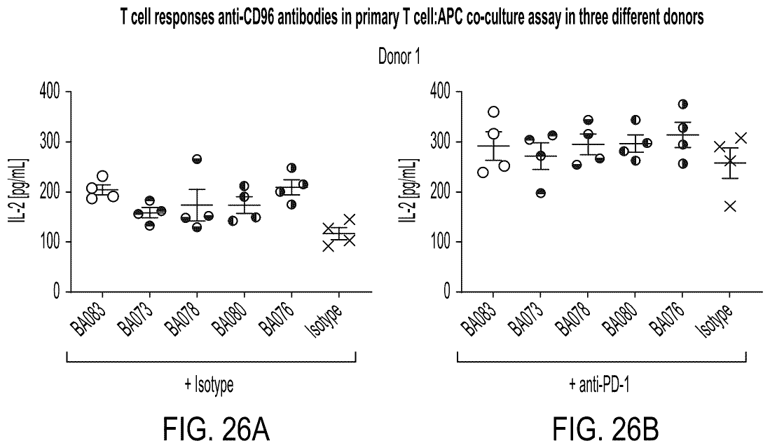

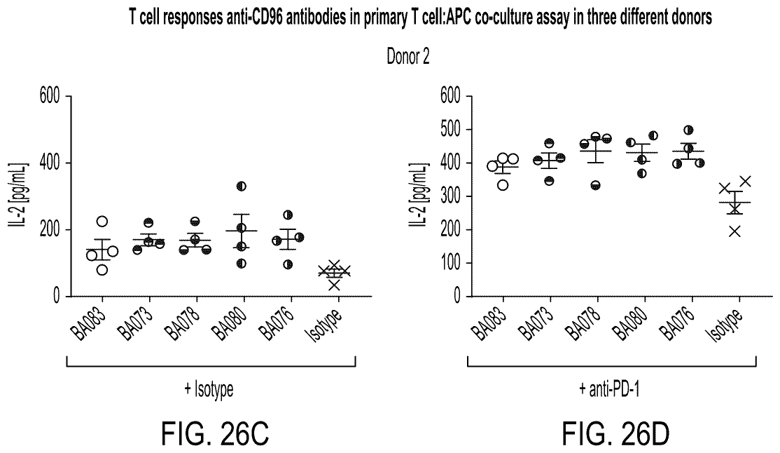

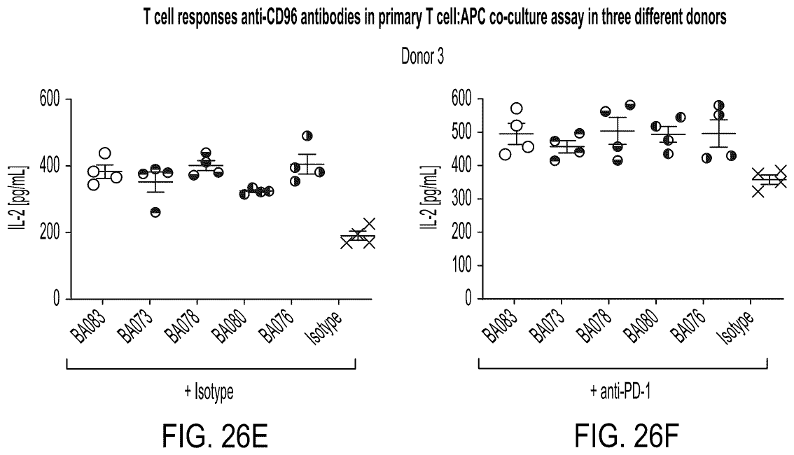

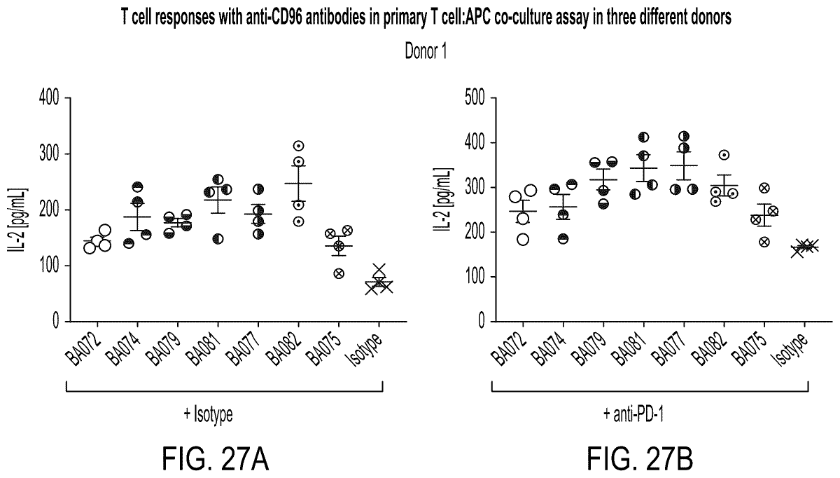

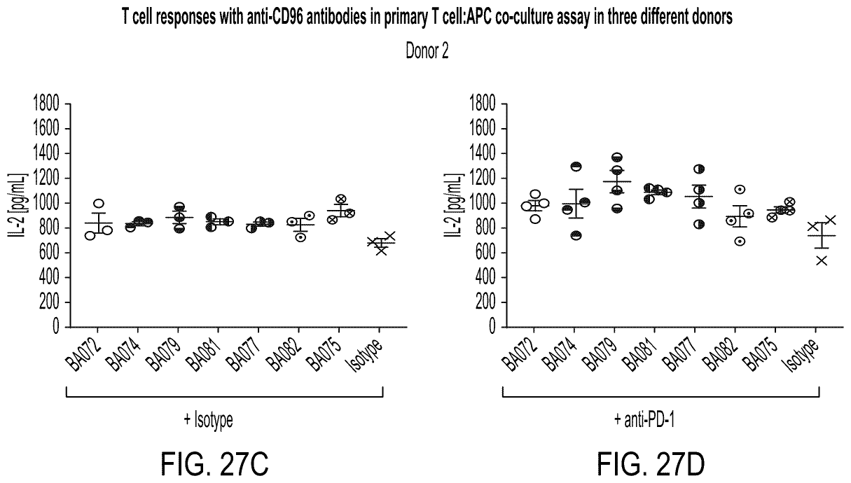

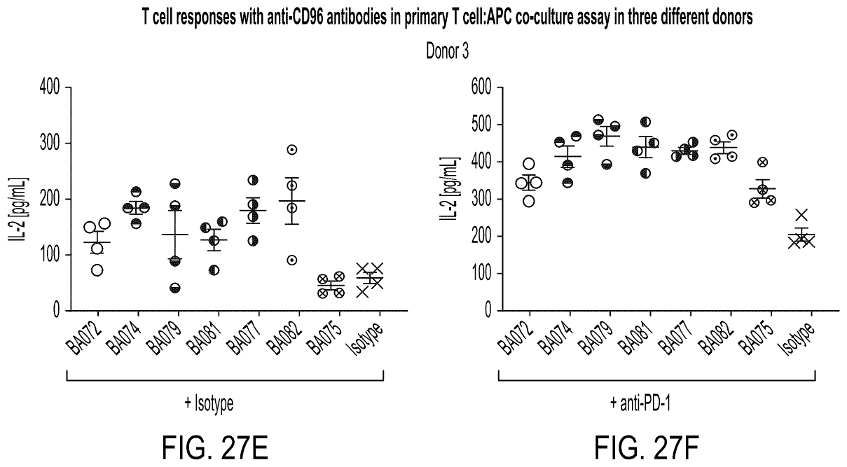

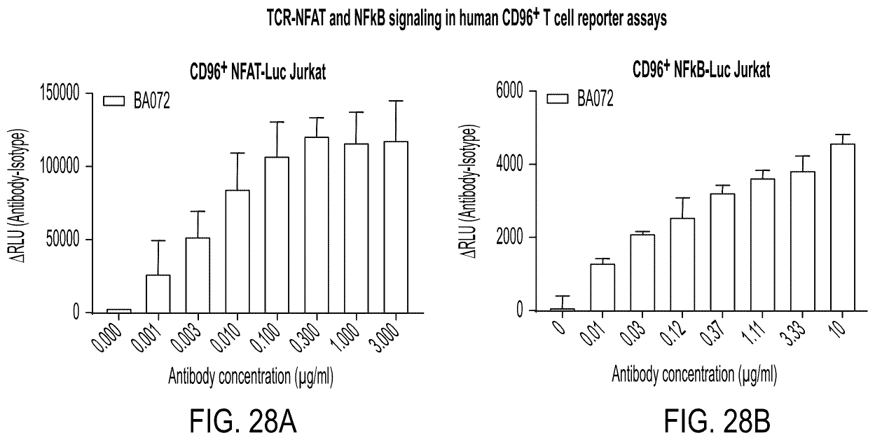

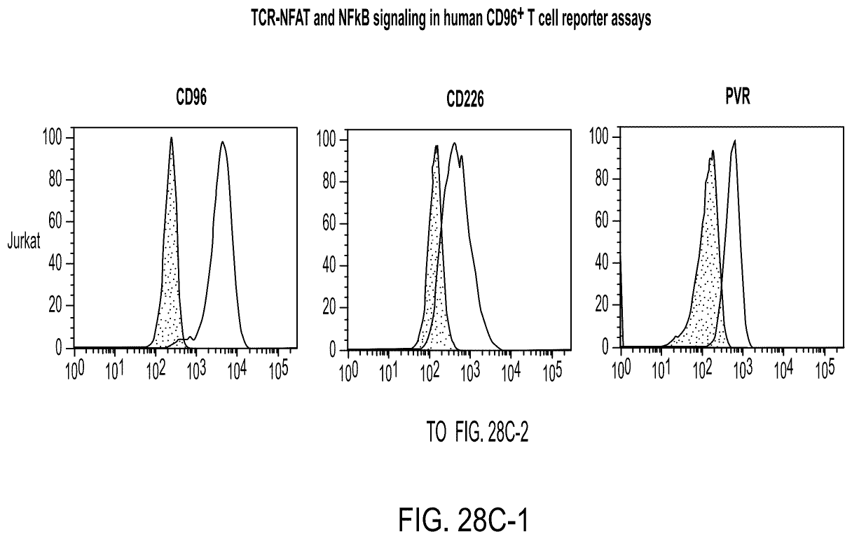

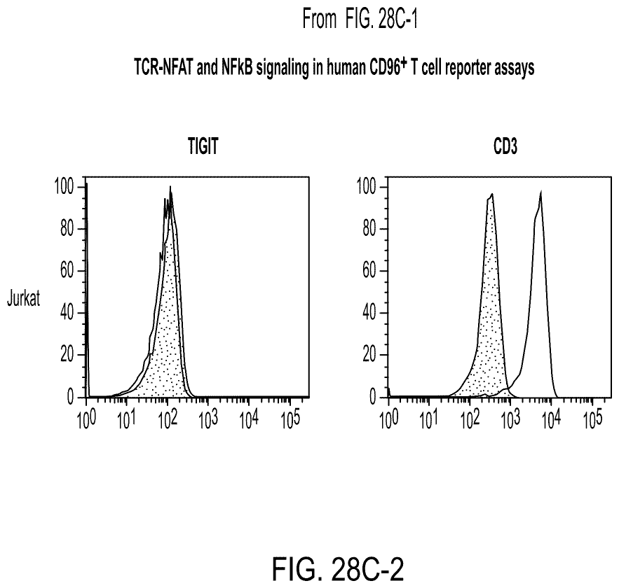

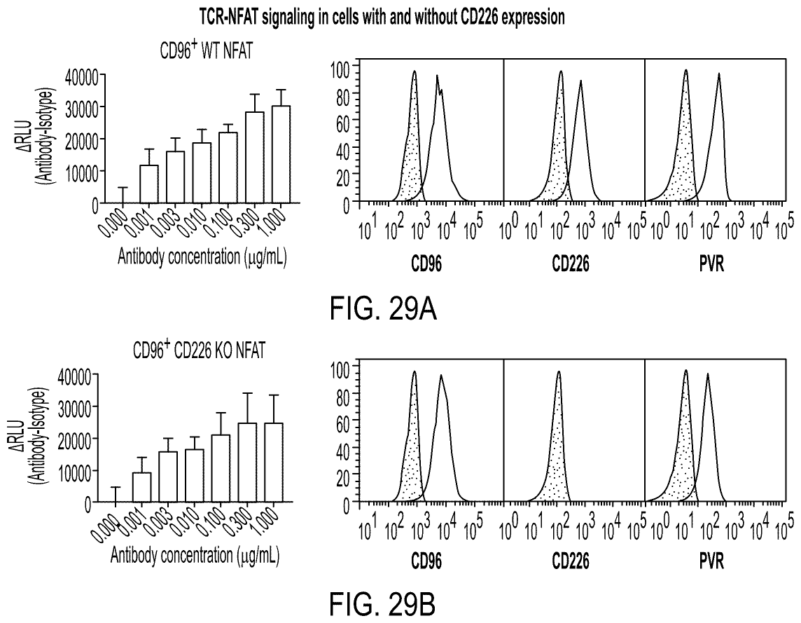

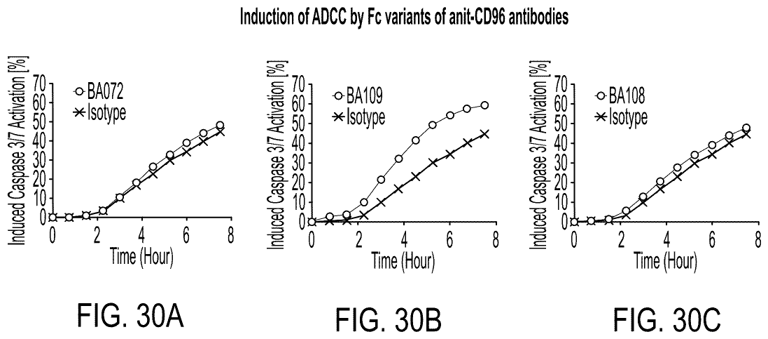

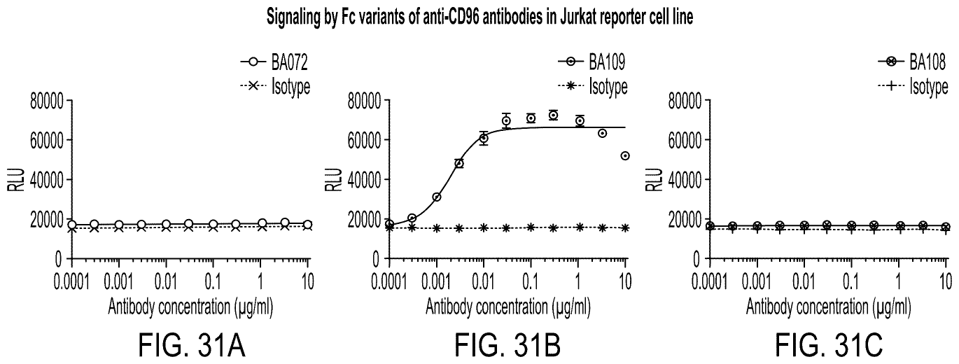

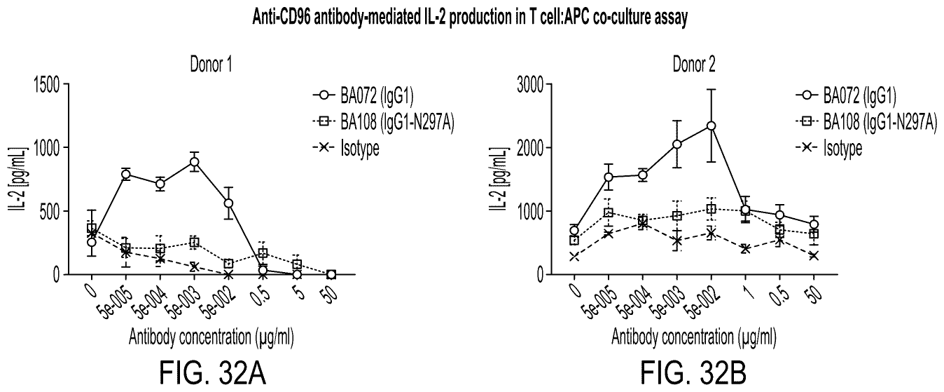

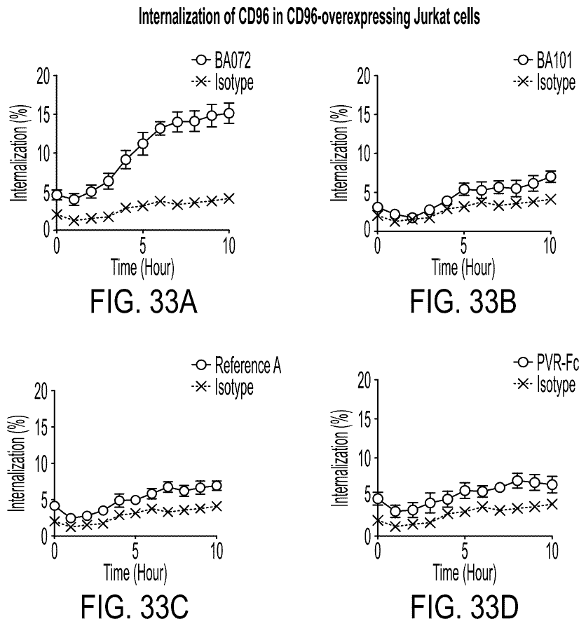

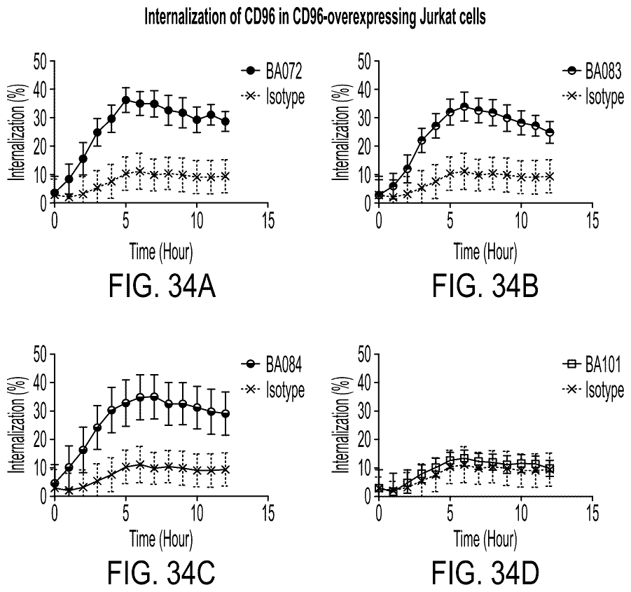

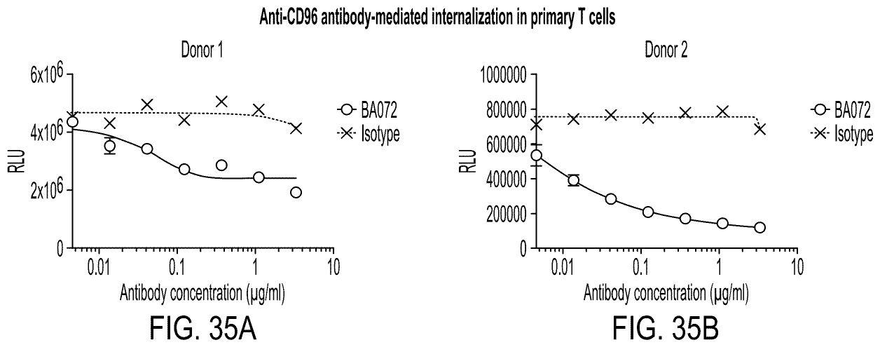

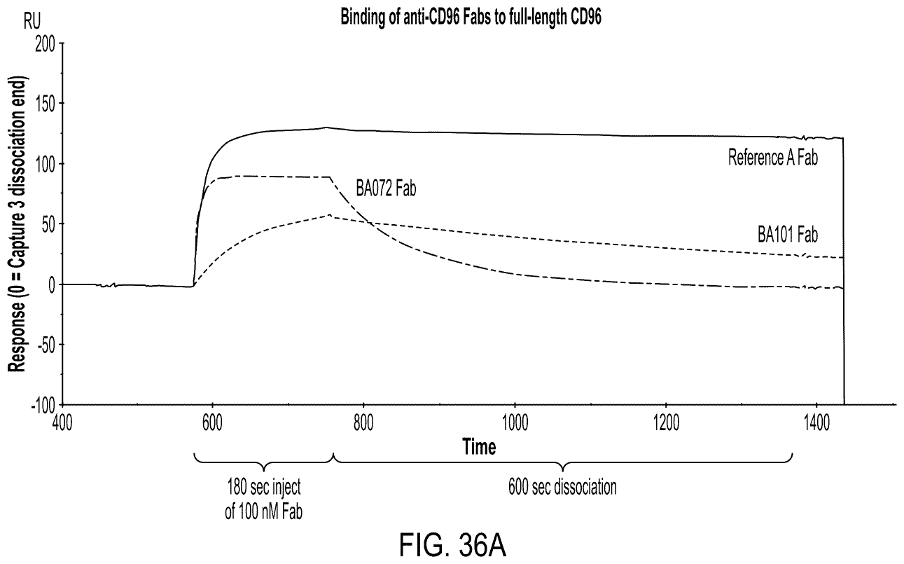

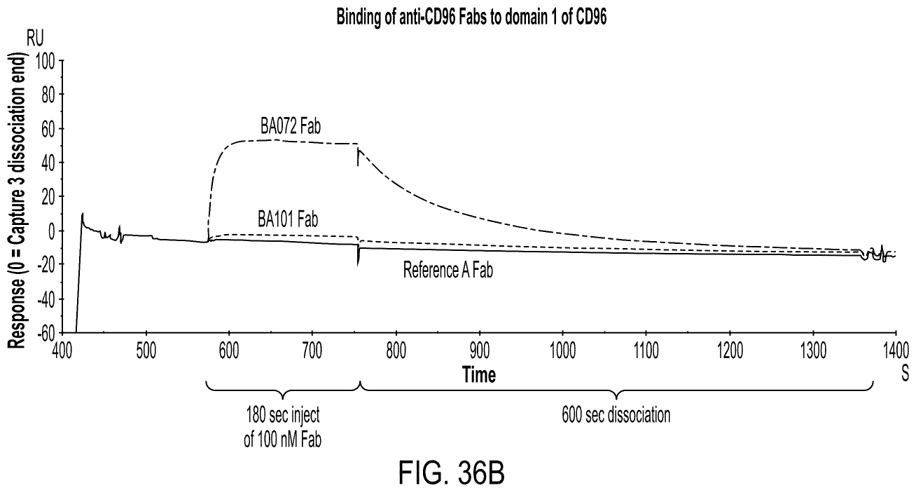

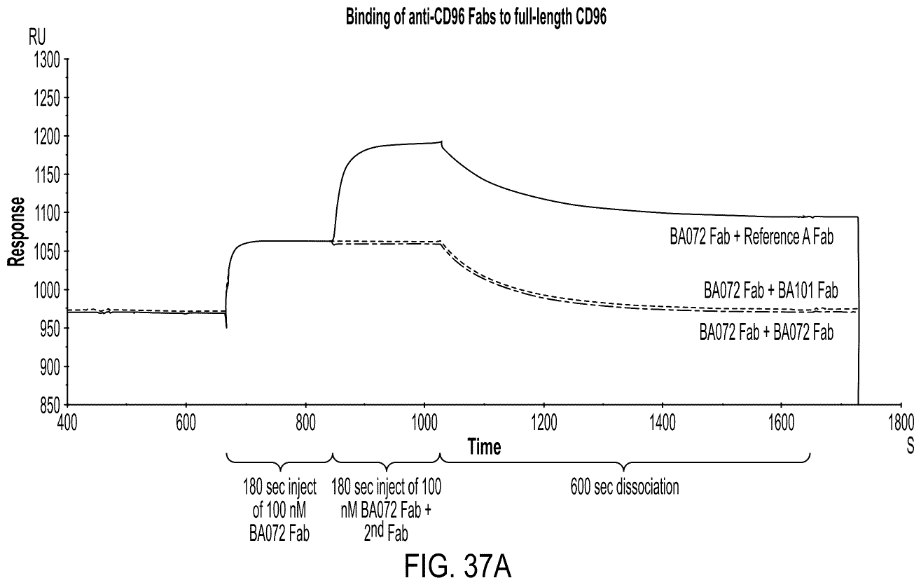

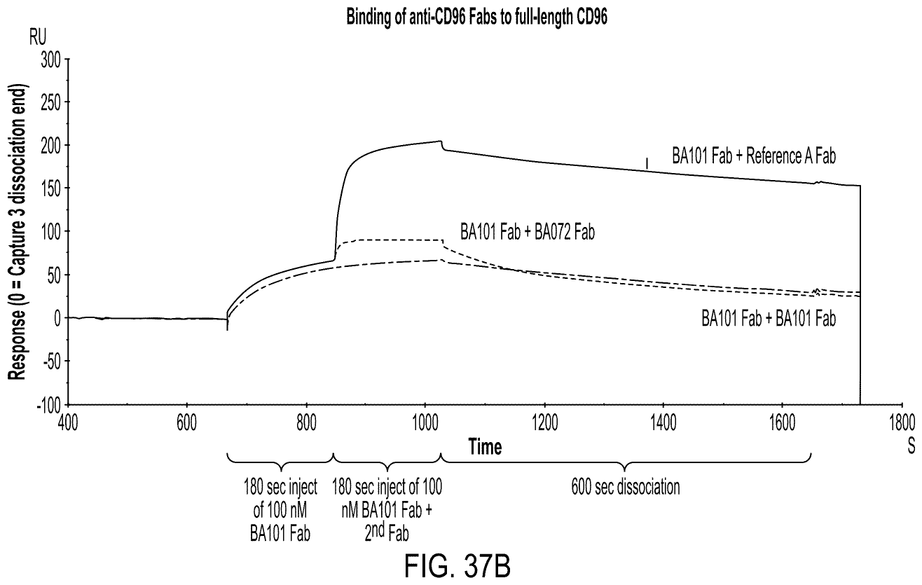

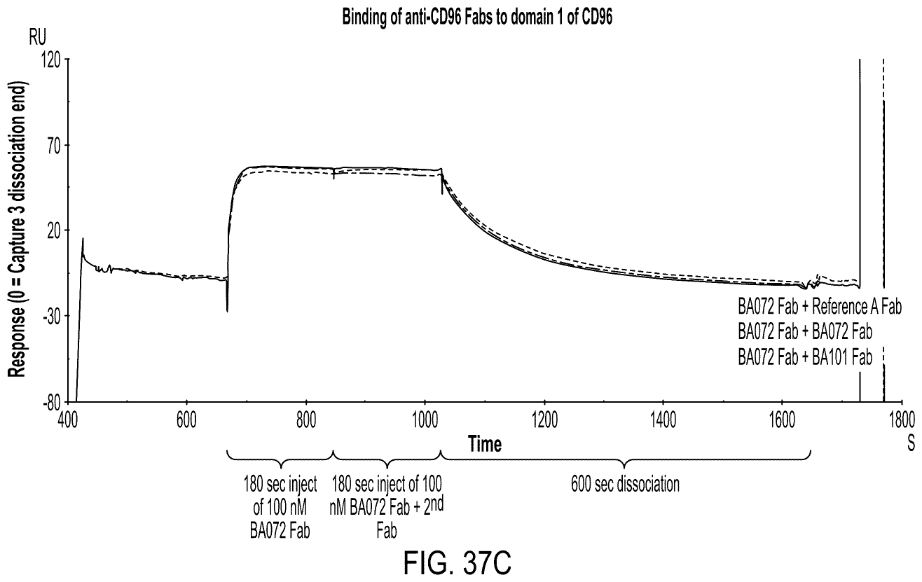

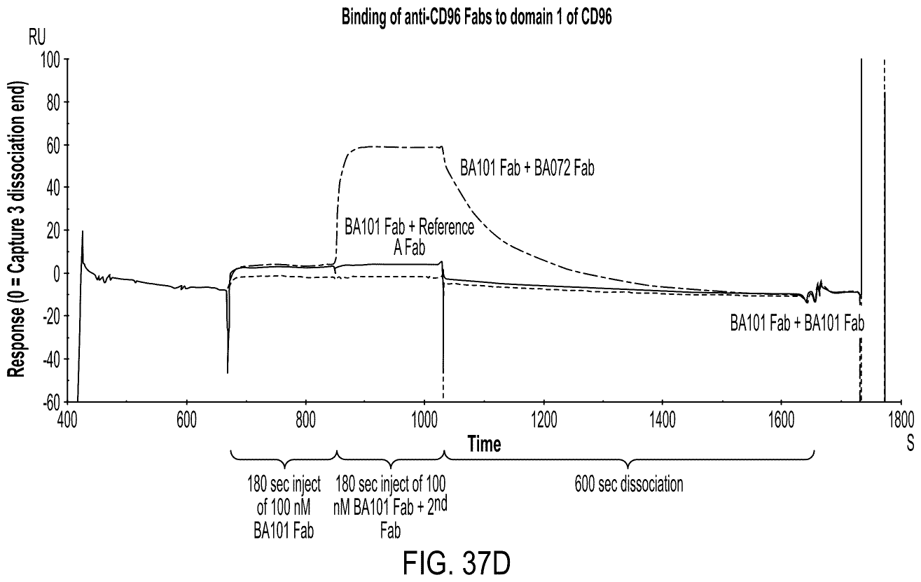

### 5. DETAILED DESCRIPTION

The instant disclosure provides antibodies that specifically bind to CD96 (e.g., human CD96 or cynomolgus CD96) and antagonize CD96 function, e.g., CD96-mediated immune suppression. Also provided are pharmaceutical compositions comprising these antibodies, nucleic acids encoding these antibodies, expression vectors and host cells for making these antibodies, and methods of treating a subject using these antibodies. The antibodies disclosed herein are particularly useful for increasing immune cell activation, and hence, are useful for treating cancer in a subject or treating or preventing an infectious disease in a subject. All instances of “isolated antibodies” described herein are additionally contemplated as antibodies that may be, but need not be, isolated. All instances of “isolated polynucleotides” described herein are additionally contemplated as polynucleotides that may be, but need not be, isolated. All instances of “antibodies” described herein are additionally contemplated as antibodies that may be, but need not be, isolated. All instances of “polynucleotides” described herein are additionally contemplated as polynucleotides that may be, but need not be, isolated.

### 5.1 Definitions

As used herein, the terms “about” and “approximately,” when used to modify a numeric value or numeric range, indicate that deviations of 5% to 10% above (e.g., up to 5% to 10% above) and 5% to 10% below (e.g., up to 5% to 10% below) the value or range remain within the intended meaning of the recited value or range.

As used herein, the term “CD96” refers to Cluster of Differentiation 96, also known as TACTILE (T cell-activation, increased late expression), that in humans is encoded by the CD96 gene. As used herein, the term “human CD96” refers to a CD96 protein encoded by a wild-type human CD96 gene (e.g., GenBank™ accession number NM_005816.5), a fragment, or a variant thereof. Exemplary extracellular portions of human CD96 are provided herein as SEQ ID NOs: 127, 128, 129, 130, and 131. Exemplary extracellular portions of cynomolgus CD96 are provided herein as SEQ ID NOs: 132, 133, and 134.

As used herein, the terms “CD155”, “polio virus receptor”, and “PVR” are used interchangeably and refer to a CD155 protein encoded by a CD155 gene (e.g., GenBank™ accession number NM_006505.5), a fragment, or a variant thereof.

As used herein, the terms “antibody” and “antibodies” include full-length antibodies, antigen-binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies, affibodies, Fab fragments, F(ab′)₂ fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA or IgY), any class (e.g., IgG1, IgG2, IgG3, IgG4, IgA1 or IgA2), or any subclass (e.g., IgG2a or IgG2b) of immunoglobulin molecule. In certain embodiments, antibodies described herein are IgG antibodies, or a class (e.g., human IgG1 or IgG4) or subclass thereof. In a specific embodiment, the antibody is a humanized monoclonal antibody. In another specific embodiment, the antibody is a human monoclonal antibody.

As used herein, the terms “VH region” and “VL region” refer, respectively, to single antibody heavy and light chain variable regions, comprising FR (Framework Regions) 1, 2, 3 and 4 and CDR (Complementarity Determining Regions) 1, 2 and 3 (see Kabat et al., (1991) Sequences of Proteins of Immunological Interest (NIH Publication No. 91-3242, Bethesda), which is herein incorporated by reference in its entirety).

As used herein, the term “CDR” or “complementarity determining region” means the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), by Chothia et al., J. Mol. Biol. 196:901-917 (1987), and by MacCallum et al., J. Mol. Biol. 262:732-745 (1996), all of which are herein incorporated by reference in their entireties, where the definitions include overlapping or subsets of amino acid residues when compared against each other. In certain embodiments, the term “CDR” is a CDR as defined by MacCallum et al., J. Mol. Biol. 262:732-745 (1996) and Martin A. “Protein Sequence and Structure Analysis of Antibody Variable Domains,” in Antibody Engineering, Kontermann and Dübel. eds., Chapter 31. pp. 422-439. Springer-Verlag, Berlin (2001). In certain embodiments, the term “CDR” is a CDR as defined by Kabat et al., J. Biol. Chem. 252, 6609-6616 (1977) and Kabat et al., Sequences of protein of immunological interest. (1991). In certain embodiments, heavy chain CDRs and light chain CDRs of an antibody are defined using different conventions. In certain embodiments, heavy chain CDRs and/or light chain CDRs are defined by performing structural analysis of an antibody and identifying residues in the variable region(s) predicted to make contact with an epitope region of a target molecule (e.g., human and/or cynomolgus CD96). CDRH1 CDRH2 and CDRH3 denote the heavy chain CDRs, and CDRL1, CDRL2 and CDRL3 denote the light chain CDRs.

As used herein, the term “framework (FR) amino acid residues” refers to those amino acids in the framework region of an immunoglobulin chain. The term “framework region” or “FR region” as used herein, includes the amino acid residues that are part of the variable region, but are not part of the CDRs (e.g., using the Kabat or MacCallum definition of CDRs).

As used herein, the terms “variable region” and “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VL” and “VL domain” are used interchangeably to refer to the light chain variable region of an antibody.

The terms “VH” and “VH domain” are used interchangeably to refer to the heavy chain variable region of an antibody.

As used herein, the terms “constant region” and “constant domain” are interchangeable and are common in the art. The constant region is an antibody portion, e.g., a carboxyl terminal portion of a light and/or heavy chain, which is not directly involved in binding of an antibody to antigen but which can exhibit various effector functions, such as interaction with an Fc receptor (e.g., Fc gamma receptor).

As used herein, the term “heavy chain” when used in reference to an antibody can refer to any distinct type, e.g., alpha (α), delta (δ), epsilon (ε), gamma (γ), and mu (μ), based on the amino acid sequence of the constant domain, which give rise to IgA, IgD, IgE, IgG, and IgM classes of antibodies, respectively, including subclasses of IgG, e.g., IgG1, IgG2, IgG3, and IgG4.

As used herein, the term “light chain” when used in reference to an antibody can refer to any distinct type, e.g., kappa (κ) or lambda (λ), based on the amino acid sequence of the constant domains. Light chain amino acid sequences are well known in the art. In specific embodiments, the light chain is a human light chain.

As used herein, the term “EU numbering system” refers to the EU numbering convention for the constant regions of an antibody, as described in Edelman, G. M. et al., Proc. Natl. Acad. USA, 63, 78-85 (1969) and Kabat et al, Sequences of Proteins of Immunological Interest, U.S. Dept. Health and Human Services, 5th edition, 1991, each of which is herein incorporated by reference in its entirety.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g., an antibody to an antigen, and k_{off} refers to the dissociation rate constant of, e.g., an antibody to an antigen. The kₒₙ and k_{off} can be determined by techniques known to one of ordinary skill in the art, such as BIAcore® or KinExA. As used herein, a “lower affinity” refers to a larger K_{D}.

As used herein, the terms “specifically binds,” “specifically recognizes,” “immunospecifically binds.” and “immunospecifically recognizes” are analogous terms in the context of antibodies and refer to molecules that bind to an antigen (e.g., epitope or immune complex) as such binding is understood by one skilled in the art. For example, a molecule that specifically binds to an antigen can bind to other peptides or polypeptides, generally with lower affinity as determined by, e.g., immunoassays, BIAcore®, KinExA 3000 instrument (Sapidyne Instruments, Boise, ID), or other assays known in the art. In a specific embodiment, molecules that specifically bind to an antigen bind to the antigen with a K_{A} that is at least 2 logs (e.g., factors of 10), 2.5 logs, 3 logs, 4 logs or greater than the K_{A} when the molecules bind non-specifically to another antigen.

In another specific embodiment, molecules that specifically bind to an antigen do not cross react with other proteins under similar binding conditions. In another specific embodiment, molecules that specifically bind to CD96 do not cross react with other non-CD96 proteins. In a specific embodiment, provided herein is an antibody that binds to CD96 (e.g., human CD96) with higher affinity than to another unrelated antigen. In certain embodiments, provided herein is an antibody that binds to CD96 (e.g., human CD96) with a 20%, 25%, 30%, 35%, 40%, 45%, 50%, 55%, 60%, 65%, 70%, 75%, 80%, 85%, 90%, 95% or higher affinity than to another, unrelated antigen as measured by, e.g., a radioimmunoassay, surface plasmon resonance, or kinetic exclusion assay. In a specific embodiment, the extent of binding of an anti-CD96 antibody described herein to an unrelated, non-CD96 protein is less than 10%, 15%, or 20% of the binding of the antibody to CD96 protein as measured by, e.g., a radioimmunoassay.

As used herein, an “epitope” is a term in the art and refers to a region of an antigen to which an antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain embodiments, the epitope to which an antibody binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays (e.g., constraining peptides using CLIPS (Chemical Linkage of Peptides onto Scaffolds) to map discontinuous or conformational epitopes), and/or mutagenesis mapping (e.g., site-directed mutagenesis mapping). For X-ray crystallography, crystallization may be accomplished using any of the known methods in the art (e.g., Giegé R et al., (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251: 6300-6303, each of which is herein incorporated by reference in its entirety). Antibody: antigen crystals may be studied using well known X-ray diffraction techniques and may be refined using computer software such as X-PLOR (Yale University, 1992, distributed by Molecular Simulations, Inc.; see, e.g., Meth Enzymol (1985) volumes 114 & 115, eds Wyckoff H W et al.; U.S. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al., (2000) Acta Crystallogr D Biol Crystallogr 56(Pt 10): 1316-1323), each of which is herein incorporated by reference in its entirety. Mutagenesis mapping studies may be accomplished using any method known to one of skill in the art. See, e.g., Champe M et al., (1995) J Biol Chem 270: 1388-1394 and Cunningham B C & Wells J A (1989) Science 244: 1081-1085, each of which is herein incorporated by reference in its entirety, for a description of mutagenesis techniques, including alanine scanning mutagenesis techniques. CLIPS (Chemical Linkage of Peptides onto Scaffolds) is a technology to present one or more peptides in a structurally constrained configuration to behave as functional mimics of complex protein domains. See, e.g., U.S. Publication Nos. US 2008/0139407 A1 and US 2007/099240 A1, and U.S. Pat. No. 7,972,993, each of which is herein incorporated by reference in its entirety. In a specific embodiment, the epitope of an antibody is determined using alanine scanning mutagenesis studies. In a specific embodiment, the epitope of an antibody is determined using hydrogen/deuterium exchange coupled with mass spectrometry. In a specific embodiment, the epitope of an antibody is determined using CLIPS Epitope Mapping Technology from Pepscan Therapeutics. In a specific embodiment, the epitope of an antibody is determined by protein mutagenesis, e.g., by generating switch mutants of an antigen with portions of its ortholog from another species and then testing the switch mutants for loss of antibody binding (e.g., by a FACS-based cell binding assay, as described herein).

As used herein, the terms “T cell receptor” and “TCR” are used interchangeably and refer to full-length heterodimeric αβ or γΔ TCRs, antigen-binding fragments of full-length TCRs, and molecules comprising TCR CDRs or variable regions. Examples of TCRs include, but are not limited to, full-length TCRs, antigen-binding fragments of full-length TCRs, soluble TCRs lacking transmembrane and cytoplasmic regions, single-chain TCRs containing variable regions of TCRs attached by a flexible linker, TCR chains linked by an engineered disulfide bond, monospecific TCRs, multi-specific TCRs (including bispecific TCRs), TCR fusions, human TCRs, humanized TCRs, chimeric TCRs, recombinantly produced TCRs, and synthetic TCRs. The term encompasses wild-type TCRs and genetically engineered TCRs (e.g., a chimeric TCR comprising a chimeric TCR chain which includes a first portion from a TCR of a first species and a second portion from a TCR of a second species).

As used herein, the terms “major histocompatibility complex” and “MHC” are used interchangeably and refer to an MHC class I molecule and/or an MHC class II molecule.

As used herein, the term “peptide-MHC complex” refers to an MHC molecule (MHC class I or MHC class II) with a peptide bound in the art-recognized peptide binding pocket of the MHC.

As used herein, the term “treat,” “treating,” and “treatment” refer to therapeutic or preventative measures described herein. The methods of “treatment” employ administration of an antibody to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “effective amount” in the context of the administration of a therapy to a subject refers to the amount of a therapy that achieves a desired prophylactic or therapeutic effect.

As used herein, the term “internalization” or ‘internalized” refers to the uptake of an antibody into an intracellular compartment of a cell upon binding of the antibody to an antigen expressed at the surface of the cell.

As used herein, the term “subject” includes any human or non-human animal. In one embodiment, the subject is a human or non-human mammal. In one embodiment, the subject is a human.

The determination of “percent identity” between two sequences (e.g., amino acid sequences or nucleic acid sequences) can be accomplished using a mathematical algorithm. A specific, non-limiting example of a mathematical algorithm utilized for the comparison of two sequences is the algorithm of Karlin S & Altschul S F (1990) PNAS 87: 2264-2268, modified as in Karlin S & Altschul S F (1993) PNAS 90: 5873-5877, each of which is herein incorporated by reference in its entirety. Such an algorithm is incorporated into the NBLAST and XBLAST programs of Altschul S F et al., (1990) J Mol Biol 215: 403, which is herein incorporated by reference in its entirety. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25: 3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

### 5.2 Anti-CD96 Antibodies

In one aspect, the instant disclosure provides antibodies that specifically bind to CD96 (e.g., human CD96 or cynomolgus CD96) and antagonize CD96 function. The amino acid sequences of exemplary antibodies are set forth in Table 1, herein.

TABLE 1
Amino acid sequences of exemplary anti-CD96 antibodies.

| | | SEQ ID |
|---|---|---|
| Description | Amino Acid Sequence | NO: |
| CDRH1 consensus | X₁YX₂X₃X₄, wherein | 135 |
| sequence 1 | X₁ is Q or S; | |
| | X₂ is A or S; | |
| | X₃ is M or I; and | |
| | X₄ is S or H. | |
| CDRH1 consensus | X₁YX₂MH, wherein | 136 |
| sequence 2 | X₁ is Q or S; and | |
| | X₂ is A or S. | |
| CDRH2 consensus | X₁IX₂X₃X₄X₅X₆X₇X₈X₉YX₁₀QKFQG, wherein | 137 |
| sequence 1 | X₁ is W or G; | |
| | X₂ is N or I; | |
| | X₃ is A, E, V, or P; | |
| | X₄ is V, G, W, or I; | |
| | X₅ is S, Y, T, N, or F; | |
| | X₆ is G or W; | |
| | X₇ is D, Y, N, or T; | |
| | X₈ is T or A; | |
| | X₉ is K or N; and | |
| | X₁₀ is S or A. | |
| CDRH2 consensus | WINX₁X₂X₃X₄X₅TKYSQKFQG, wherein | 138 |
| sequence 2 | X₁ is A, V, or E; | |
| | X₂ is V, W, or G; | |
| | X₃ is S, Y, T, or N; | |
| | X₄ is G or W; and | |
| | X₅ is D, N, Y, or T. | |
| CDRH3 consensus | NWGX₁SYGX₂DV, wherein | 180 |
| sequence | X₁ is M or L; and | |
| | X₂ is M or L. | |
| CDRL1 consensus | RASQSIX₁X₂YLN, wherein | 139 |
| sequence | X₁ is S, T, or L; and | |
| | X₂ is S, P, or W. | |
| CDRL2 consensus | X₁X₂SSLQS, wherein | 141 |
| sequence | X₁ is S or A; and | |
| | X₂ is A, S, or E. | |
| CDRL3 consensus | QQX₁YSTPALX₂, wherein | 143 |
| sequence | X₁ is S or A; and | |
| | X₂ is T or S. | |
| CDRH1 - BA072, | SYAMH | 1 |
| BA083, BA081, BA080, | | |
| BA084, BA085, BA086, | | |
| BA087, BA088, BA089, | | |
| BA090, BA091, BA093, | | |
| BA094, BA095, BA096, | | |
| BA097, BA098, BA099, | | |
| BA100 | | |
| CDRH1 - BA074, | QYAMH | 2 |
| BA073, BA075, BA077, | | |
| BA076, BA079, BA078, | | |
| BA082 | | |
| CDRH1 - BA092 | SYSMH | 3 |
| CDRH1 - BA101, | SYAIS | 4 |
| BA102, BA103, BA104, | | |
| BA105, BA106, BA107 | | |
| CDRH2 - BA072, | WINAGNGNTKYSQKFQG | 5 |
| BA083, BA084, BA085, | | |
| BA086, BA087, BA088, | | |
| BA094, BA095, BA096, | | |
| BA097, BA098, BA099, | | |
| BA100 | | |
| CDRH2 - BA074, | WINAVSGDTKYSQKFQG | 6 |
| BA073 | | |
| CDRH2 - BA081, | WINAGTGDTKYSQKFQG | 7 |
| BA080 | | |
| CDRH2 - BA075 | WINEGYGNTKYSQKFQG | 8 |
| CDRH2 - BA077, | WINAGYGYTKYSQKFQG | 9 |
| BA076 | | |
| CDRH2 - BA079, | WINAGTGNTKYSQKFQG | 10 |
| BA078 | | |
| CDRH2 - BA082 | WINAGYGNTKYSQKFQG | 11 |
| CDRH2 - BA089 | WINAWNGNTKYSQKFQG | 12 |
| CDRH2 - BA090 | WINVGTGTTKYSQKFQG | 13 |
| CDRH2 - BA091 | WINAVNGNTKYSQKFQG | 14 |
| CDRH2 - BA092 | WINAGNWNTKYSQKFQG | 15 |
| CDRH2 - BA093 | WINAWTGNTKYSQKFQG | 16 |
| CDRH2 - BA101, | GIIPIFGTANYAQKFQG | 17 |
| BA102, BA103, BA104, | | |
| BA105, BA106, BA107 | | |
| CDRH3 - BA072, | NWGMSYGMDV | 18 |
| BA083, BA074, BA073, | | |
| BA081, BA080, BA075, | | |
| BA77, BA076, BA079, | | |
| BA78, BA082, BA084, | | |
| BA085, BA086, BA087, | | |
| BA088, BA089, BA090, | | |
| BA091, BA092, BA093, | | |
| BA094 | | |
| CDRH3 - BA095, | NWGMSYGLDV | 140 |
| BA098 | | |
| CDRH3 - BA096, | NWGLSYGMDV | 142 |
| BA099 | | |
| CDRH3 - BA097, | NWGLSYGLDV | 179 |
| BA100 | | |
| CDRH3 - BA101, | GYDSRPLDV | 19 |
| BA102, BA103, BA104, | | |
| BA105, BA106 | | |
| CDRH3 - BA107 | GYDSRPLDY | 20 |
| CDRL1 - BA072, | RASQSISSYLN | 21 |
| BA083, BA074, BA073, | | |
| BA081, BA080, BA075, | | |
| BA77, BA076, BA079, | | |
| BA78, BA082, BA084, | | |
| BA088, BA089, BA090, | | |
| BA091, BA092, BA093, | | |
| BA095, BA096, BA097, | | |
| BA098, BA099, BA100 | | |
| CDRL1 - BA085 | RASQSISPYLN | 22 |
| CDRL1 - BA087 | RASQSILSYLN | 23 |
| CDRL1 - BA086 | RASQSISWYLN | 24 |
| CDRL1 - BA094 | RASQSITSYLN | 25 |
| CDRL1 - BA101, | GGNNIGSKIVH | 26 |
| BA102, BA103, BA104, | | |
| BA105, BA106, BA107 | | |
| CDRL2 - BA072, | AASSLQS | 28 |
| BA083, BA084, BA089, | | |
| BA090, BA091, BA092, | | |
| BA093, BA094, BA095, | | |
| BA096, BA097, BA098, | | |
| BA099, BA100 | | |
| CDRL2 - BA074, | SASSLQS | 29 |
| BA073, BA081, BA080, | | |
| BA075, BA079, BA078, | | |
| BA085, BA086, BA087, | | |
| BA088 | | |
| CDRL2 - BA077, BA076 | SESSLQS | 30 |
| CDRL2 - BA082 | SSSSLQS | 31 |
| CDRL2 - BA101, | DDRDRPS | 32 |
| BA102, BA103, BA104, | | |
| BA105, BA106, BA107 | | |
| CDRL3 - BA072, | QQSYSTPALT | 33 |
| BA083, BA074, BA073, | | |
| BA081, BA080, BA075, | | |
| BA77, BA076, BA079, | | |
| BA78, BA082, BA084, | | |
| BA085, BA086, BA087, | | |
| BA088, BA089, BA090, | | |
| BA091, BA092, BA093, | | |
| BA095, BA096, BA097, | | |
| BA098, BA099, BA100 | | |
| CDRL3 - BA094 | QQAYSTPALS | 34 |
| CDRL3 - BA101, | QVWDINVHHVI | 35 |
| BA102, BA103, BA104, | | |
| BA105, BA106, BA107 | | |
| VH - BA072 | XVQLVQSGAEVKKPGASVKVSCKASGYTFTSYA | 36 |
| | MHWVRQAPGQRLEWMGWINAGNGNTKYSQKFQ | |
| | GRVTITRDTSTSTAYMELRSLRSDDTAMYYCARN | |
| | WGMSYGMDVWGQGTMVTVSS, wherein X is | |
| | glutamine (Q) or pyroglutamate (pE) | |
| VH - BA083, BA085, | XVQLVQSGAEVKKPGASVKVSCKASGYTFTSYA | 37 |
| BA086, BA087, BA088, | MHWVRQAPGQRLEWMGWINAGNGNTKYSQKFQ | |
| BA094 | GRVTITRDTSASTAYMELSSLRSEDTAVYYCARN | |
| | WGMSYGMDVWGQGTTVTVSS, wherein X is | |
| | glutamine (Q) or pyroglutamate (pE) | |
| VH - BA074 | XVQLVQSGAEVKKPGASVKVSCKASGYTFTQYA | 38 |
| | MHWVRQAPGQRLEWMGWINAVSGDTKYSQKFQ | |
| | GRVTITRDTSTSTAYMELRSLRSDDTAMYYCARN | |
| | WGMSYGMDVWGQGTMVTVSS, wherein X is | |
| | glutamine (Q) or pyroglutamate (pE) | |
| VH - BA073 | XVQLVQSGAEVKKPGASVKVSCKASGYTFTQYA | 39 |
| | MHWVRQAPGQRLEWMGWINAVSGDTKYSQKFQ | |
| | GRVTITRDTSASTAYMELSSLRSEDTAVYYCARN | |
| | WGMSYGMDVWGQGTTVTVSS, wherein X is | |
| | glutamine (Q) or pyroglutamate (pE) | |
| VH - BA081 | XVQLVQSGAEVKKPGASVKVSCKASGYTFTSYA | 40 |
| | MHWVRQAPGQRLEWMGWINAGTGDTKYSQKFQ | |
| | GRVTITRDTSTSTAYMELRSLRSDDTAMYYCARN | |
| | WGMSYGMDVWGQGTMVTVSS, wherein X is | |
| | glutamine (Q) or pyroglutamate (pE) | |
| VH - BA080 | XVQLVQSGAEVKKPGASVKVSCKASGYTFTSYA | 41 |
| | MHWVRQAPGQRLEWMGWINAGTGDTKYSQKFQ | |
| | GRVTITRDTSASTAYMELSSLRSEDTAVYYCARN | |
| | WGMSYGMDVWGQGTTVTVSS, wherein X is | |
| | glutamine (Q) or pyroglutamate (pE) | |
| VH - BA075 | XVQLVQSGAEVKKPGASVKVSCKASGYTFNQYA | 42 |
| | MHWVRQAPGQRLEWMGWINEGYGNTKYSQKFQ | |
| | GRVTITRDTSTSTAYMELRSLRSDDTAMYYCARN | |
| | WGMSYGMDVWGQGTMVTVSS, wherein X is | |
| | glutamine (Q) or pyroglutamate (pE) | |
| VH - BA077 | XVQLVQSGAEVKKPGASVKVSCKASGYTFTQYA | 43 |
| | MHWVRQAPGQRLEWMGWINAGYGYTKYSQKFQ | |
| | GRVTITRDTSTSTAYMELRSLRSDDTAMYYCARN | |
| | WGMSYGMDVWGQGTMVTVSS, wherein X is | |
| | glutamine (Q) or pyroglutamate (pE) | |
| VH - BA076 | XVQLVQSGAEVKKPGASVKVSCKASGYTFTQYA | 44 |
| | MHWVRQAPGQRLEWMGWINAGYGYTKYSQKFQ | |
| | GRVTITRDTSASTAYMELSSLRSEDTAVYYCARN | |
| | WGMSYGMDVWGQGTTVTVSS, wherein X is | |
| | glutamine (Q) or pyroglutamate (pE) | |
| VH - BA079 | XVQLVQSGAEVKKPGASVKVSCKASGYTFSQYA | 45 |
| | MHWVRQAPGQRLEWMGWINAGTGNTKYSQKFQ | |
| | GRVTITRDTSTSTAYMELRSLRSDDTAMYYCARN | |
| | WGMSYGMDVWGQGTMVTVSS, wherein X is | |
| | glutamine (Q) or pyroglutamate (pE) | |
| VH - BA078 | XVQLVQSGAEVKKPGASVKVSCKASGYTFSQYA | 46 |
| | MHWVRQAPGQRLEWMGWINAGTGNTKYSQKFQ | |
| | GRVTITRDTSASTAYMELSSLRSEDTAVYYCARN | |
| | WGMSYGMDVWGQGTTVTVSS, wherein X is | |
| | glutamine (Q) or pyroglutamate (pE) | |
| VH - BA082 | XVQLVQSGAEVKKPGASVKVSCKASGYTFDQYA | 47 |
| | MHWVRQAPGQRLEWMGWINAGYGNTKYSQKFQ | |
| | GRVTITRDTSTSTAYMELRSLRSDDTAMYYCARN | |
| | WGMSYGMDVWGQGTMVTVSS, wherein X is | |
| | glutamine (Q) or pyroglutamate (pE) | |
| VH - BA084 | XVQLVQSGAEVKKPGASVKVSCKASGYTFTSYA | 48 |
| | MHWVRQAPGQRLEWMGWINAGNGNTKYSQKFQ | |
| | GRVTITRDTSTSTAYMELRSLRSDDTAVYYCARN | |
| | WGMSYGMDVWGQGTTVTVSS, wherein X is | |
| | glutamine (Q) or pyroglutamate (pE) | |
| VH - BA089 | XVQLVQSGAEVKKPGASVKVSCKASGYTFTSYA | 49 |
| | MHWVRQAPGQRLEWMGWINAWNGNTKYSQKF | |
| | QGRVTITRDTSASTAYMELSSLRSEDTAVYYCAR | |
| | NWGMSYGMDVWGQGTTVTVSS, wherein X is | |
| | glutamine (Q) or pyroglutamate (pE) | |
| VH - BA090 | XVQLVQSGAEVKKPGASVKVSCKASGYTFTSYA | 50 |
| | MHWVRQAPGQRLEWMGWINVGTGTTKYSQKFQ | |
| | GRVTITRDTSASTAYMELSSLRSEDTAVYYCARN | |
| | WGMSYGMDVWGQGTTVTVSS, wherein X is | |
| | glutamine (Q) or pyroglutamate (pE) | |
| VH - BA091 | XVQLVQSGAEVKKPGASVKVSCKASGYTFSSYA | 51 |
| | MHWVRQAPGQRLEWMGWINAVNGNTKYSQKFQ | |
| | GRVTITRDTSASTAYMELSSLRSEDTAVYYCARN | |
| | WGMSYGMDVWGQGTTVTVSS, wherein X is | |
| | glutamine (Q) or pyroglutamate (pE) | |
| VH - BA092 | XVQLVQSGAEVKKPGASVKVSCKASGYTFASYS | 52 |
| | MHWVRQAPGQRLEWMGWINAGNWNTKYSQKF | |
| | QGRVTITRDTSASTAYMELSSLRSEDTAVYYCAR | |
| | NWGMSYGMDVWGQGTTVTVSS, wherein X is | |
| | glutamine (Q) or pyroglutamate (pE) | |
| VH - BA093 | XVQLVQSGAEVKKPGASVKVSCKASGYTFTSYA | 53 |
| | MHWVRQAPGQRLEWMGWINAWTGNTKYSQKF | |
| | QGRVTITRDTSASTAYMELSSLRSEDTAVYYCAR | |
| | NWGMSYGMDVWGQGTTVTVSS, wherein X is | |
| | glutamine (Q) or pyroglutamate (pE) | |
| VH - BA095 | XVQLVQSGAEVKKPGASVKVSCKASGYTFTSYA | 54 |
| | MHWVRQAPGQRLEWMGWINAGNGNTKYSQKFQ | |
| | GRVTITRDTSTSTAYMELRSLRSDDTAMYYCARN | |
| | WGMSYGLDVWGQGTMVTVSS, wherein X is | |
| | glutamine (Q) or pyroglutamate (pE) | |
| VH - BA096 | XVQLVQSGAEVKKPGASVKVSCKASGYTFTSYA | 55 |
| | MHWVRQAPGQRLEWMGWINAGNGNTKYSQKFQ | |
| | GRVTITRDTSTSTAYMELRSLRSDDTAMYYCARN | |
| | WGLSYGMDVWGQGTMVTVSS, wherein X is | |
| | glutamine (Q) or pyroglutamate (pE) | |
| VH - BA097 | XVQLVQSGAEVKKPGASVKVSCKASGYTFTSYA | 56 |
| | MHWVRQAPGQRLEWMGWINAGNGNTKYSQKFQ | |
| | GRVTITRDTSTSTAYMELRSLRSDDTAMYYCARN | |
| | WGLSYGLDVWGQGTMVTVSS, wherein X is | |
| | glutamine (Q) or pyroglutamate (pE) | |
| VH - BA098 | XVQLVQSGAEVKKPGASVKVSCKASGYTFTSYA | 57 |
| | MHWVRQAPGQRLEWMGWINAGNGNTKYSQKFQ | |
| | GRVTITRDTSASTAYMELSSLRSEDTAVYYCARN | |
| | WGMSYGLDVWGQGTTVTVSS, wherein X is | |
| | glutamine (Q) or pyroglutamate (pE) | |
| VH - BA099 | XVQLVQSGAEVKKPGASVKVSCKASGYTFTSYA | 58 |
| | MHWVRQAPGQRLEWMGWINAGNGNTKYSQKFQ | |
| | GRVTITRDTSASTAYMELSSLRSEDTAVYYCARN | |
| | WGLSYGMDVWGQGTTVTVSS, wherein X is | |
| | glutamine (Q) or pyroglutamate (pE) | |
| VH - BA100 | XVQLVQSGAEVKKPGASVKVSCKASGYTFTSYA | 59 |
| | MHWVRQAPGQRLEWMGWINAGNGNTKYSQKFQ | |
| | GRVTITRDTSASTAYMELSSLRSEDTAVYYCARN | |
| | WGLSYGLDVWGQGTTVTVSS, wherein X is | |
| | glutamine (Q) or pyroglutamate (pE) | |
| VH - BA101, BA102, | XVQLVQSGAEVKKPGSSVKVSCKASGGTFSSYAIS | 60 |
| BA103, BA104, BA105, | WVRQAPGQGLEWMGGIIPIFGTANYAQKFQGRVT | |
| BA106 | ITADKSTSTAYMELSSLRSEDTAVYYCARGYDSRP | |
| | LDVWGQGTLVTVSS, wherein X is glutamine (Q) or | |
| | pyroglutamate (pE) | |
| VH - BA107 | XVQLVQSGAEVKKPGSSVKVSCKASGGTFSSYAIS | 61 |
| | WVRQAPGQGLEWMGGIIPIFGTANYAQKFQGRVT | |
| | ITADKSTSTAYMELSSLRSEDTAVYYCARGYDSRP | |
| | LDYWGQGTLVTVSS, wherein X is glutamine (Q) or | |
| | pyroglutamate (pE) | |
| VL - BA072, BA083, | DIQMTQSPSSLSASVGDRVTITCRASQSISSYLNWY | 62 |
| BA084, BA089, BA090, | QQKPGKAPKLLIYAASSLQSGVPSRFSGSGSGTDF | |
| BA091, BA092, BA093 | TLTISSLQPEDFATYYCQQSYSTPALTFGGGTKVDI | |
| | K | |
| VL - BA074, BA073, | DIQMTQSPSSLSASVGDRVTITCRASQSISSYLNWY | 63 |
| BA081, BA080, BA075, | QQKPGKAPKLLIYSASSLQSGVPSRFSGSGSGTDFT | |
| BA079, BA078, BA088 | LTISSLQPEDFATYYCQQSYSTPALTFGGGTKVDIK | |
| VL - BA077, BA076 | DIQMTQSPSSLSASVGDRVTITCRASQSISSYLNWY | 64 |
| | QQKPGKAPKLLIYSESSLQSGVPSRFSGSGSGTDFT | |
| | LTISSLQPEDFATYYCQQSYSTPALTFGGGTKVDIK | |
| VL - BA082 | DIQMTQSPSSLSASVGDRVTITCRASQSISSYLNWY | 65 |
| | QQKPGKAPKLLIYSSSSLQSGVPSRFSGSGSGTDFT | |
| | LTISSLQPEDFATYYCQQSYSTPALTFGGGTKVDIK | |
| VL - BA085 | DIQMTQSPSSLSASVGDRVTITCRASQSISPYLNWY | 66 |
| | QQKPGKAPKLLIYSASSLQSGVPSRFSGSGSGTDFT | |
| | LTISSLQPEDFATYYCQQSYSTPALTFGGGTKVDIK | |
| VL - BA086 | DIQMTQSPSSLSASVGDRVTITCRASQSISWYLNW | 67 |
| | YQQKPGKAPKLLIYSASSLQSGVPSRFSGSGSGTD | |
| | FTLTISSLQPEDFATYYCQQSYSTPALTFGGGTKV | |
| | DIK | |
| VL - BA087 | DIQMTQSPSSLSASVGDRVTITCRASQSILSYLNW | 68 |
| | YQQKPGKAPKLLIYSASSLQSGVPSRFSGSGSGTD | |
| | FTLTISSLQPEDFATYYCQQSYSTPALTFGGGTKV | |
| | DIK | |
| VL - BA094 | DIQMTQSPSSLSASVGDRVTITCRASQSITSYLNW | 69 |
| | YQQKPGKAPKLLIYAASSLQSGVPSRFSGSGSGTD | |
| | FTLTISSLQPEDFATYYCQQAYSTPALSFGGGTKV | |
| | DIK | |
| VL - BA101, BA107 | SYELTQPLSVSVALGQTASITCGGNNIGSKIVHWY | 70 |
| | QQKSGQAPVLVVSDDRDRPSGIPERFSGSNSGNTA | |
| | TLTINTVEAGDEADYYCQVWDINVHHVIFGGGTK | |
| | VTVL | |
| VL - BA102 | SYELTQPLSVSVALGQTASITCGGNNIGSKIVHWY | 71 |
| | QQKPGQAPVLVVSDDRDRPSGIPERFSGSNSGNTA | |
| | TLTISRVEAGDEADYYCQVWDINVHHVIFGGGTK | |
| | LTVL | |
| VL - BA103 | SYELTQPLSVSVALGQTASITCGGNNIGSKIVHWY | 72 |
| | QQKSGQAPVLVIYDDRDRPSGIPERFSGSNSGNTA | |
| | TLTINTVEAGDEADYYCQVWDINVHHVIFGGGTK | |
| | LTVL | |
| VL - BA104 | SYELTQPLSVSVALGQTASITCGGNNIGSKIVHWY | 73 |
| | QQKPGQAPVLVVSDDRDRPSGIPERFSGSNSGNTA | |
| | TLTINTVEAGDEADYYCQVWDINVHHVIFGGGTK | |
| | LTVL | |
| VL - BA105 | SYELTQPLSVSVALGQTASITCGGNNIGSKIVHWY | 74 |
| | QQKSGQAPVLVVSDDRDRPSGIPERFSGSNSGNTA | |
| | TLTISRVEAGDEADYYCQVWDINVHHVIFGGGTK | |
| | LTVL | |
| VL - BA106 | SYELTQPLSVSVALGQTASITCGGNNIGSKIVHWY | 75 |
| | QQKSGQAPVLVVSDDRDRPSGIPERFSGSNSGNTA | |
| | TLTISRAQAGDEADYYCQVWDINVHHVIFGGGTK | |
| | LTVL | |
| full-length heavy chain | XVQLVQSGAEVKKPGASVKVSCKASGYTFTSYA | 76 |
| (with C-terminal lysine) - | MHWVRQAPGQRLEWMGWINAGNGNTKYSQKFQ | |
| BA072 | GRVTITRDTSTSTAYMELRSLRSDDTAMYYCARN | |
| | WGMSYGMDVWGQGTMVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPGK, wherein X is glutamine (Q) or | |
| | pyroglutamate (pE) | |
| full-length heavy chain | XVQLVQSGAEVKKPGASVKVSCKASGYTFTSYA | 144 |
| (without C-terminal | MHWVRQAPGQRLEWMGWINAGNGNTKYSQKFQ | |
| lysine) - BA072 | GRVTITRDTSTSTAYMELRSLRSDDTAMYYCARN | |
| | WGMSYGMDVWGQGTMVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPG, wherein X is glutamine (Q) or | |
| | pyroglutamate (pE) | |
| full-length heavy chain | XVQLVQSGAEVKKPGASVKVSCKASGYTFTSYA | 77 |
| (with C-terminal lysine) - | MHWVRQAPGQRLEWMGWINAGNGNTKYSQKFQ | |
| BA083, BA085, BA086, | GRVTITRDTSASTAYMELSSLRSEDTAVYYCARN | |
| BA087, BA088, BA094 | WGMSYGMDVWGQGTTVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPGK, wherein X is glutamine (Q) or | |
| | pyroglutamate (pE) | |
| full-length heavy chain | XVQLVQSGAEVKKPGASVKVSCKASGYTFTSYA | 145 |
| (without C-terminal | MHWVRQAPGQRLEWMGWINAGNGNTKYSQKFQ | |
| lysine) - BA083, BA085, | GRVTITRDTSASTAYMELSSLRSEDTAVYYCARN | |
| BA086, BA087, BA088, | WGMSYGMDVWGQGTTVTVSSASTKGPSVFPLAP | |
| BA094 | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPG, wherein X is glutamine (Q) or | |
| | pyroglutamate (pE) | |
| full-length heavy chain | XVQLVQSGAEVKKPGASVKVSCKASGYTFTQYA | 78 |
| (with C-terminal lysine) - | MHWVRQAPGQRLEWMGWINAVSGDTKYSQKFQ | |
| BA074 | GRVTITRDTSTSTAYMELRSLRSDDTAMYYCARN | |
| | WGMSYGMDVWGQGTMVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPGK, wherein X is glutamine (Q) or | |
| | pyroglutamate (pE) | |
| full-length heavy chain | XVQLVQSGAEVKKPGASVKVSCKASGYTFTQYA | 146 |
| (without C-terminal | MHWVRQAPGQRLEWMGWINAVSGDTKYSQKFQ | |
| lysine) - BA074 | GRVTITRDTSTSTAYMELRSLRSDDTAMYYCARN | |
| | WGMSYGMDVWGQGTMVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPG, wherein X is glutamine (Q) or | |
| | pyroglutamate (pE) | |
| full-length heavy chain | XVQLVQSGAEVKKPGASVKVSCKASGYTFTQYA | 79 |
| (with C-terminal lysine) - | MHWVRQAPGQRLEWMGWINAVSGDTKYSQKFQ | |
| BA073 | GRVTITRDTSASTAYMELSSLRSEDTAVYYCARN | |
| | WGMSYGMDVWGQGTTVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPGK, wherein X is glutamine (Q) or | |
| | pyroglutamate (pE) | |
| full-length heavy chain | XVQLVQSGAEVKKPGASVKVSCKASGYTFTQYA | 147 |
| (without C-terminal | MHWVRQAPGQRLEWMGWINAVSGDTKYSQKFQ | |
| lysine) - BA073 | GRVTITRDTSASTAYMELSSLRSEDTAVYYCARN | |
| | WGMSYGMDVWGQGTTVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPG, wherein X is glutamine (Q) or | |
| | pyroglutamate (pE) | |
| full-length heavy chain | XVQLVQSGAEVKKPGASVKVSCKASGYTFTSYA | 80 |
| (with C-terminal lysine) - | MHWVRQAPGQRLEWMGWINAGTGDTKYSQKFQ | |
| BA081 | GRVTITRDTSTSTAYMELRSLRSDDTAMYYCARN | |
| | WGMSYGMDVWGQGTMVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPGK, wherein X is glutamine (Q) or | |
| | pyroglutamate (pE) | |
| full-length heavy chain | XVQLVQSGAEVKKPGASVKVSCKASGYTFTSYA | 148 |
| (without C-terminal | MHWVRQAPGQRLEWMGWINAGTGDTKYSQKFQ | |
| lysine) - BA081 | GRVTITRDTSTSTAYMELRSLRSDDTAMYYCARN | |
| | WGMSYGMDVWGQGTMVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPG, wherein X is glutamine (Q) or | |
| | pyroglutamate (pE) | |
| full-length heavy chain | XVQLVQSGAEVKKPGASVKVSCKASGYTFTSYA | 81 |
| (with C-terminal lysine) - | MHWVRQAPGQRLEWMGWINAGTGDTKYSQKFQ | |
| BA080 | GRVTITRDTSASTAYMELSSLRSEDTAVYYCARN | |
| | WGMSYGMDVWGQGTTVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPGK, wherein X is glutamine (Q) or | |
| | pyroglutamate (pE) | |
| full-length heavy chain | XVQLVQSGAEVKKPGASVKVSCKASGYTFTSYA | 149 |
| (without C-terminal | MHWVRQAPGQRLEWMGWINAGTGDTKYSQKFQ | |
| lysine) - BA080 | GRVTITRDTSASTAYMELSSLRSEDTAVYYCARN | |
| | WGMSYGMDVWGQGTTVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPG, wherein X is glutamine (Q) or | |
| | pyroglutamate (pE) | |
| full-length heavy chain | XVQLVQSGAEVKKPGASVKVSCKASGYTFNQYA | 82 |
| (with C-terminal lysine) - | MHWVRQAPGQRLEWMGWINEGYGNTKYSQKFQ | |
| BA075 | GRVTITRDTSTSTAYMELRSLRSDDTAMYYCARN | |
| | WGMSYGMDVWGQGTMVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPGK, wherein X is glutamine (Q) or | |
| | pyroglutamate (pE) | |
| full-length heavy chain | XVQLVQSGAEVKKPGASVKVSCKASGYTFNQYA | 150 |
| (without C-terminal | MHWVRQAPGQRLEWMGWINEGYGNTKYSQKFQ | |
| lysine) - BA075 | GRVTITRDTSTSTAYMELRSLRSDDTAMYYCARN | |
| | WGMSYGMDVWGQGTMVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPG, wherein X is glutamine (Q) or | |
| | pyroglutamate (pE) | |
| full-length heavy chain | XVQLVQSGAEVKKPGASVKVSCKASGYTFTQYA | 83 |
| (with C-terminal lysine) - | MHWVRQAPGQRLEWMGWINAGYGYTKYSQKFQ | |
| BA077 | GRVTITRDTSTSTAYMELRSLRSDDTAMYYCARN | |
| | WGMSYGMDVWGQGTMVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPGK, wherein X is glutamine (Q) or | |
| | pyroglutamate (pE) | |
| full-length heavy chain | XVQLVQSGAEVKKPGASVKVSCKASGYTFTQYA | 151 |
| (without C-terminal | MHWVRQAPGQRLEWMGWINAGYGYTKYSQKFQ | |
| lysine) - BA077 | GRVTITRDTSTSTAYMELRSLRSDDTAMYYCARN | |
| | WGMSYGMDVWGQGTMVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPG, wherein X is glutamine (Q) or | |
| | pyroglutamate (pE) | |
| full-length heavy chain | XVQLVQSGAEVKKPGASVKVSCKASGYTFTQYA | 84 |
| (with C-terminal lysine) - | MHWVRQAPGQRLEWMGWINAGYGYTKYSQKFQ | |
| BA076 | GRVTITRDTSASTAYMELSSLRSEDTAVYYCARN | |
| | WGMSYGMDVWGQGTTVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPGK, wherein X is glutamine (Q) or | |
| | pyroglutamate (pE) | |
| full-length heavy chain | XVQLVQSGAEVKKPGASVKVSCKASGYTFTQYA | 152 |
| (without C-terminal | MHWVRQAPGQRLEWMGWINAGYGYTKYSQKFQ | |
| lysine) - BA076 | GRVTITRDTSASTAYMELSSLRSEDTAVYYCARN | |
| | WGMSYGMDVWGQGTTVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPG, wherein X is glutamine (Q) or | |
| | pyroglutamate (pE) | |
| full-length heavy chain | XVQLVQSGAEVKKPGASVKVSCKASGYTFSQYA | 85 |
| (with C-terminal lysine) - | MHWVRQAPGQRLEWMGWINAGTGNTKYSQKFQ | |
| BA079 | GRVTITRDTSTSTAYMELRSLRSDDTAMYYCARN | |
| | WGMSYGMDVWGQGTMVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPGK, wherein X is glutamine (Q) or | |
| | pyroglutamate (pE) | |
| full-length heavy chain | XVQLVQSGAEVKKPGASVKVSCKASGYTFSQYA | 153 |
| (without C-terminal | MHWVRQAPGQRLEWMGWINAGTGNTKYSQKFQ | |
| lysine) - BA079 | GRVTITRDTSTSTAYMELRSLRSDDTAMYYCARN | |
| | WGMSYGMDVWGQGTMVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPG, wherein X is glutamine (Q) or | |
| | pyroglutamate (pE) | |
| full-length heavy chain | XVQLVQSGAEVKKPGASVKVSCKASGYTFSQYA | 86 |
| (with C-terminal lysine) - | MHWVRQAPGQRLEWMGWINAGTGNTKYSQKFQ | |
| BA078 | GRVTITRDTSASTAYMELSSLRSEDTAVYYCARN | |
| | WGMSYGMDVWGQGTTVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPGK, wherein X is glutamine (Q) or | |
| | pyroglutamate (pE) | |
| full-length heavy chain | XVQLVQSGAEVKKPGASVKVSCKASGYTFSQYA | 154 |
| (without C-terminal | MHWVRQAPGQRLEWMGWINAGTGNTKYSQKFQ | |
| lysine) - BA078 | GRVTITRDTSASTAYMELSSLRSEDTAVYYCARN | |
| | WGMSYGMDVWGQGTTVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPG, wherein X is glutamine (Q) or | |
| | pyroglutamate (pE) | |
| full-length heavy chain | XVQLVQSGAEVKKPGASVKVSCKASGYTFDQYA | 87 |
| (with C-terminal lysine) - | MHWVRQAPGQRLEWMGWINAGYGNTKYSQKFQ | |
| BA082 | GRVTITRDTSTSTAYMELRSLRSDDTAMYYCARN | |
| | WGMSYGMDVWGQGTMVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPGK, wherein X is glutamine (Q) or | |
| | pyroglutamate (pE) | |
| full-length heavy chain | XVQLVQSGAEVKKPGASVKVSCKASGYTFDQYA | 155 |
| (without C-terminal | MHWVRQAPGQRLEWMGWINAGYGNTKYSQKFQ | |
| lysine) - BA082 | GRVTITRDTSTSTAYMELRSLRSDDTAMYYCARN | |
| | WGMSYGMDVWGQGTMVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPG, wherein X is glutamine (Q) or | |
| | pyroglutamate (pE) | |
| full-length heavy chain | XVQLVQSGAEVKKPGASVKVSCKASGYTFTSYA | 88 |
| (with C-terminal lysine) - | MHWVRQAPGQRLEWMGWINAGNGNTKYSQKFQ | |
| BA084 | GRVTITRDTSTSTAYMELRSLRSDDTAVYYCARN | |
| | WGMSYGMDVWGQGTTVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPGK, wherein X is glutamine (Q) or | |
| | pyroglutamate (pE) | |
| full-length heavy chain | XVQLVQSGAEVKKPGASVKVSCKASGYTFTSYA | 156 |
| (without C-terminal | MHWVRQAPGQRLEWMGWINAGNGNTKYSQKFQ | |
| lysine) - BA084 | GRVTITRDTSTSTAYMELRSLRSDDTAVYYCARN | |
| | WGMSYGMDVWGQGTTVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPG, wherein X is glutamine (Q) or | |
| | pyroglutamate (pE) | |
| full-length heavy chain | XVQLVQSGAEVKKPGASVKVSCKASGYTFTSYA | 89 |
| (with C-terminal lysine) - | MHWVRQAPGQRLEWMGWINAWNGNTKYSQKF | |
| BA089 | QRVTITRDTSASTAYMELSSLRSEDTAVYYCAR | |
| | NWGMSYGMDVWGQGTTVTVSSASTKGPSVFPLA | |
| | PSSKSTSGGTAALGCLVKDYFPEPVTVSWNSGAL | |
| | TSGVHTFPAVLQSSGLYSLSSVVTVPSSSLGTQTYI | |
| | CNVNHKPSNTKVDKRVEPKSCDKTHTCPPCPAPE | |
| | LLGGPSVFLFPPKPKDTLMISRTPEVTCVVVDVSH | |
| | EDPEVKFNWYVDGVEVHNAKTKPREEQYNSTYR | |
| | VVSVLTVLHQDWLNGKEYKCKVSNKALPAPIEKT | |
| | ISKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPGK, wherein X is glutamine (Q) or | |
| | pyroglutamate (pE) | |
| full-length heavy chain | XVQLVQSGAEVKKPGASVKVSCKASGYTFTSYA | 157 |
| (without C-terminal | MHWVRQAPGQRLEWMGWINAWNGNTKYSQKF | |
| lysine) - BA089 | QGRVTITRDTSASTAYMELSSLRSEDTAVYYCAR | |
| | NWGMSYGMDVWGQGTTVTVSSASTKGPSVFPLA | |
| | PSSKSTSGGTAALGCLVKDYFPEPVTVSWNSGAL | |
| | TSGVHTFPAVLQSSGLYSLSSVVTVPSSSLGTQTYI | |
| | CNVNHKPSNTKVDKRVEPKSCDKTHTCPPCPAPE | |
| | LLGGPSVFLFPPKPKDTLMISRTPEVTCVVVDVSH | |
| | EDPEVKFNWYVDGVEVHNAKTKPREEQYNSTYR | |
| | VVSVLTVLHQDWLNGKEYKCKVSNKALPAPIEKT | |
| | ISKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPG, wherein X is glutamine (Q) or | |
| | pyroglutamate (pE) | |
| full-length heavy chain | XVQLVQSGAEVKKPGASVKVSCKASGYTFTSYA | 90 |
| (with C-terminal lysine) - | MHWVRQAPGQRLEWMGWINVGTGTTKYSQKFQ | |
| BA090 | GRVTITRDTSASTAYMELSSLRSEDTAVYYCARN | |
| | WGMSYGMDVWGQGTTVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPGK, wherein X is glutamine (Q) or | |
| | pyroglutamate (pE) | |
| full-length heavy chain | XVQLVQSGAEVKKPGASVKVSCKASGYTFTSYA | 158 |
| (without C-terminal | MHWVRQAPGQRLEWMGWINVGTGTTKYSQKFQ | |
| lysine) - BA090 | GRVTITRDTSASTAYMELSSLRSEDTAVYYCARN | |
| | WGMSYGMDVWGQGTTVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPG, wherein X is glutamine (Q) or | |
| | pyroglutamate (pE) | |
| full-length heavy chain | XVQLVQSGAEVKKPGASVKVSCKASGYTFSSYA | 91 |
| (with C-terminal lysine) - | MHWVRQAPGQRLEWMGWINAVNGNTKYSQKFQ | |
| BA091 | GRVTITRDTSASTAYMELSSLRSEDTAVYYCARN | |
| | WGMSYGMDVWGQGTTVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPGK, wherein X is glutamine (Q) or | |
| | pyroglutamate (pE) | |
| full-length heavy chain | XVQLVQSGAEVKKPGASVKVSCKASGYTFSSYA | 159 |
| (without C-terminal | MHWVRQAPGQRLEWMGWINAVNGNTKYSQKFQ | |
| lysine) - BA091 | GRVTITRDTSASTAYMELSSLRSEDTAVYYCARN | |
| | WGMSYGMDVWGQGTTVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPG, wherein X is glutamine (Q) or | |
| | pyroglutamate (pE) | |
| full-length heavy chain | XVQLVQSGAEVKKPGASVKVSCKASGYTFASYS | 92 |
| (with C-terminal lysine) - | MHWVRQAPGQRLEWMGWINAGNWNTKYSQKF | |
| BA092 | QGRVTITRDTSASTAYMELSSLRSEDTAVYYCAR | |
| | NWGMSYGMDVWGQGTTVTVSSASTKGPSVFPLA | |
| | PSSKSTSGGTAALGCLVKDYFPEPVTVSWNSGAL | |
| | TSGVHTFPAVLQSSGLYSLSSVVTVPSSSLGTQTYI | |
| | CNVNHKPSNTKVDKRVEPKSCDKTHTCPPCPAPE | |
| | LLGGPSVFLFPPKPKDTLMISRTPEVTCVVVDVSH | |
| | EDPEVKFNWYVDGVEVHNAKTKPREEQYNSTYR | |
| | VVSVLTVLHQDWLNGKEYKCKVSNKALPAPIEKT | |
| | ISKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPGK, wherein X is glutamine (Q) or | |
| | pyroglutamate (pE) | |
| full-length heavy chain | XVQLVQSGAEVKKPGASVKVSCKASGYTFASYS | 160 |
| (without C-terminal | MHWVRQAPGQRLEWMGWINAGNWNTKYSQKF | |
| lysine) - BA092 | QGRVTITRDTSASTAYMELSSLRSEDTAVYYCAR | |
| | NWGMSYGMDVWGQGTTVTVSSASTKGPSVFPLA | |
| | PSSKSTSGGTAALGCLVKDYFPEPVTVSWNSGAL | |
| | TSGVHTFPAVLQSSGLYSLSSVVTVPSSSLGTQTYI | |
| | CNVNHKPSNTKVDKRVEPKSCDKTHTCPPCPAPE | |
| | LLGGPSVFLFPPKPKDTLMISRTPEVTCVVVDVSH | |
| | EDPEVKFNWYVDGVEVHNAKTKPREEQYNSTYR | |
| | VVSVLTVLHQDWLNGKEYKCKVSNKALPAPIEKT | |
| | ISKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPG, wherein X is glutamine (Q) or | |
| | pyroglutamate (pE) | |
| full-length heavy chain | XVQLVQSGAEVKKPGASVKVSCKASGYTFTSYA | 93 |
| (with C-terminal lysine) - | MHWVRQAPGQRLEWMGWINAWTGNTKYSQKF | |
| BA093 | QGRVTITRDTSASTAYMELSSLRSEDTAVYYCAR | |
| | NWGMSYGMDVWGQGTTVTVSSASTKGPSVFPLA | |
| | PSSKSTSGGTAALGCLVKDYFPEPVTVSWNSGAL | |
| | TSGVHTFPAVLQSSGLYSLSSVVTVPSSSLGTQTYI | |
| | CNVNHKPSNTKVDKRVEPKSCDKTHTCPPCPAPE | |
| | LLGGPSVFLFPPKPKDTLMISRTPEVTCVVVDVSH | |
| | EDPEVKFNWYVDGVEVHNAKTKPREEQYNSTYR | |
| | VVSVLTVLHQDWLNGKEYKCKVSNKALPAPIEKT | |
| | ISKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPGK, wherein X is glutamine (Q) or | |
| | pyroglutamate (pE) | |
| full-length heavy chain | XVQLVQSGAEVKKPGASVKVSCKASGYTFTSYA | 161 |
| (without C-terminal | MHWVRQAPGQRLEWMGWINAWTGNTKYSQKF | |
| lysine) - BA093 | QGRVTITRDTSASTAYMELSSLRSEDTAVYYCAR | |
| | NWGMSYGMDVWGQGTTVTVSSASTKGPSVFPLA | |
| | PSSKSTSGGTAALGCLVKDYFPEPVTVSWNSGAL | |
| | TSGVHTFPAVLQSSGLYSLSSVVTVPSSSLGTQTYI | |
| | CNVNHKPSNTKVDKRVEPKSCDKTHTCPPCPAPE | |
| | LLGGPSVFLFPPKPKDTLMISRTPEVTCVVVDVSH | |
| | EDPEVKFNWYVDGVEVHNAKTKPREEQYNSTYR | |
| | VVSVLTVLHQDWLNGKEYKCKVSNKALPAPIEKT | |
| | ISKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPG, wherein X is glutamine (Q) or | |
| | pyroglutamate (pE) | |
| full-length heavy chain | XVQLVQSGAEVKKPGASVKVSCKASGYTFTSYA | 94 |
| (with C-terminal lysine) - | MHWVRQAPGQRLEWMGWINAGNGNTKYSQKFQ | |
| BA095 | GRVTITRDTSTSTAYMELRSLRSDDTAMYYCARN | |
| | WGMSYGLDVWGQGTMVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPGK, wherein X is glutamine (Q) or | |
| | pyroglutamate (pE) | |
| full-length heavy chain | XVQLVQSGAEVKKPGASVKVSCKASGYTFTSYA | 162 |
| (without C-terminal | MHWVRQAPGQRLEWMGWINAGNGNTKYSQKFQ | |
| lysine) - BA095 | GRVTITRDTSTSTAYMELRSLRSDDTAMYYCARN | |
| | WGMSYGLDVWGQGTMVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPG, wherein X is glutamine (Q) or | |
| | pyroglutamate (pE) | |
| full-length heavy chain | XVQLVQSGAEVKKPGASVKVSCKASGYTFTSYA | 95 |
| (with C-terminal lysine) - | MHWVRQAPGQRLEWMGWINAGNGNTKYSQKFQ | |
| BA096 | GRVTITRDTSTSTAYMELRSLRSDDTAMYYCARN | |
| | WGLSYGMDVWGQGTMVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPGK, wherein X is glutamine (Q) or | |
| | pyroglutamate (pE) | |
| full-length heavy chain | XVQLVQSGAEVKKPGASVKVSCKASGYTFTSYA | 163 |
| (without C-terminal | MHWVRQAPGQRLEWMGWINAGNGNTKYSQKFQ | |
| lysine) - BA096 | GRVTITRDTSTSTAYMELRSLRSDDTAMYYCARN | |
| | WGLSYGMDVWGQGTMVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPG, wherein X is glutamine (Q) or | |
| | pyroglutamate (pE) | |
| full-length heavy chain | XVQLVQSGAEVKKPGASVKVSCKASGYTFTSYA | 96 |
| (with C-terminal lysine) - | MHWVRQAPGQRLEWMGWINAGNGNTKYSQKFQ | |
| BA097 | GRVTITRDTSTSTAYMELRSLRSDDTAMYYCARN | |
| | WGLSYGLDVWGQGTMVTVSSASTKGPSVFPLAPS | |
| | SKSTSGGTAALGCLVKDYFPEPVTVSWNSGALTS | |
| | GVHTFPAVLQSSGLYSLSSVVTVPSSSLGTQTYICN | |
| | VNHKPSNTKVDKRVEPKSCDKTHTCPPCPAPELL | |
| | GGPSVFLFPPKPKDTLMISRTPEVTCVVVDVSHED | |
| | PEVKFNWYVDGVEVHNAKTKPREEQYNSTYRVV | |
| | SVLTVLHQDWLNGKEYKCKVSNKALPAPIEKTIS | |
| | KAKGQPREPQVYTLPPSREEMTKNQVSLTCLVKG | |
| | FYPSDIAVEWESNGQPENNYKTTPPVLDSDGSFFL | |
| | YSKLTVDKSRWQQGNVFSCSVMHEALHNHYTQK | |
| | SLSLSPGK, wherein X is glutamine (Q) or | |
| | pyroglutamate (pE) | |
| full-length heavy chain | XVQLVQSGAEVKKPGASVKVSCKASGYTFTSYA | 164 |
| (without C-terminal | MHWVRQAPGQRLEWMGWINAGNGNTKYSQKFQ | |
| lysine) - BA097 | GRVTITRDTSTSTAYMELRSLRSDDTAMYYCARN | |
| | WGLSYGLDVWGQGTMVTVSSASTKGPSVFPLAPS | |
| | SKSTSGGTAALGCLVKDYFPEPVTVSWNSGALTS | |
| | GVHTFPAVLQSSGLYSLSSVVTVPSSSLGTQTYICN | |
| | VNHKPSNTKVDKRVEPKSCDKTHTCPPCPAPELL | |
| | GGPSVFLFPPKPKDTLMISRTPEVTCVVVDVSHED | |
| | PEVKFNWYVDGVEVHNAKTKPREEQYNSTYRVV | |
| | SVLTVLHQDWLNGKEYKCKVSNKALPAPIEKTIS | |
| | KAKGQPREPQVYTLPPSREEMTKNQVSLTCLVKG | |
| | FYPSDIAVEWESNGQPENNYKTTPPVLDSDGSFFL | |
| | YSKLTVDKSRWQQGNVFSCSVMHEALHNHYTQK | |
| | SLSLSPG, wherein X is glutamine (Q) or pyroglutamate | |
| | (pE) | |
| full-length heavy chain | XVQLVQSGAEVKKPGASVKVSCKASGYTFTSYA | 97 |
| (with C-terminal lysine) - | MHWVRQAPGQRLEWMGWINAGNGNTKYSQKFQ | |
| BA098 | GRVTITRDTSASTAYMELSSLRSEDTAVYYCARN | |
| | WGMSYGLDVWGQGTTVTVSSASTKGPSVFPLAPS | |
| | SKSTSGGTAALGCLVKDYFPEPVTVSWNSGALTS | |
| | GVHTFPAVLQSSGLYSLSSVVTVPSSSLGTQTYICN | |
| | VNHKPSNTKVDKRVEPKSCDKTHTCPPCPAPELL | |
| | GGPSVFLFPPKPKDTLMISRTPEVTCVVVDVSHED | |
| | PEVKFNWYVDGVEVHNAKTKPREEQYNSTYRVV | |
| | SVLTVLHQDWLNGKEYKCKVSNKALPAPIEKTIS | |
| | KAKGQPREPQVYTLPPSREEMTKNQVSLTCLVKG | |
| | FYPSDIAVEWESNGQPENNYKTTPPVLDSDGSFFL | |
| | YSKLTVDKSRWQQGNVFSCSVMHEALHNHYTQK | |
| | SLSLSPGK, wherein X is glutamine (Q) or | |
| | pyroglutamate (pE) | |
| full-length heavy chain | XVQLVQSGAEVKKPGASVKVSCKASGYTFTSYA | 165 |
| (without C-terminal | MHWVRQAPGQRLEWMGWINAGNGNTKYSQKFQ | |
| lysine) - BA098 | GRVTITRDTSASTAYMELSSLRSEDTAVYYCARN | |
| | WGMSYGLDVWGQGTTVTVSSASTKGPSVFPLAPS | |
| | SKSTSGGTAALGCLVKDYFPEPVTVSWNSGALTS | |
| | GVHTFPAVLQSSGLYSLSSVVTVPSSSLGTQTYICN | |
| | VNHKPSNTKVDKRVEPKSCDKTHTCPPCPAPELL | |
| | GGPSVFLFPPKPKDTLMISRTPEVTCVVVDVSHED | |
| | PEVKFNWYVDGVEVHNAKTKPREEQYNSTYRVV | |
| | SVLTVLHQDWLNGKEYKCKVSNKALPAPIEKTIS | |
| | KAKGQPREPQVYTLPPSREEMTKNQVSLTCLVKG | |
| | FYPSDIAVEWESNGQPENNYKTTPPVLDSDGSFFL | |
| | YSKLTVDKSRWQQGNVFSCSVMHEALHNHYTQK | |
| | SLSLSPG, wherein X is glutamine (Q) or pyroglutamate | |
| | (pE) | |
| full-length heavy chain | XVQLVQSGAEVKKPGASVKVSCKASGYTFTSYA | 98 |
| (with C-terminal lysine) - | MHWVRQAPGQRLEWMGWINAGNGNTKYSQKFQ | |
| BA099 | GRVTITRDTSASTAYMELSSLRSEDTAVYYCARN | |
| | WGLSYGMDVWGQGTTVTVSSASTKGPSVFPLAPS | |
| | SKSTSGGTAALGCLVKDYFPEPVTVSWNSGALTS | |
| | GVHTFPAVLQSSGLYSLSSVVTVPSSSLGTQTYICN | |
| | VNHKPSNTKVDKRVEPKSCDKTHTCPPCPAPELL | |
| | GGPSVFLFPPKPKDTLMISRTPEVTCVVVDVSHED | |
| | PEVKFNWYVDGVEVHNAKTKPREEQYNSTYRVV | |
| | SVLTVLHQDWLNGKEYKCKVSNKALPAPIEKTIS | |
| | KAKGQPREPQVYTLPPSREEMTKNQVSLTCLVKG | |
| | FYPSDIAVEWESNGQPENNYKTTPPVLDSDGSFFL | |
| | YSKLTVDKSRWQQGNVFSCSVMHEALHNHYTQK | |
| | SLSLSPGK, wherein X is glutamine (Q) or | |
| | pyroglutamate (pE) | |
| full-length heavy chain | XVQLVQSGAEVKKPGASVKVSCKASGYTFTSYA | 166 |
| (without C-terminal | MHWVRQAPGQRLEWMGWINAGNGNTKYSQKFQ | |
| lysine) - BA099 | GRVTITRDTSASTAYMELSSLRSEDTAVYYCARN | |
| | WGLSYGMDVWGQGTTVTVSSASTKGPSVFPLAPS | |
| | SKSTSGGTAALGCLVKDYFPEPVTVSWNSGALTS | |
| | GVHTFPAVLQSSGLYSLSSVVTVPSSSLGTQTYICN | |
| | VNHKPSNTKVDKRVEPKSCDKTHTCPPCPAPELL | |
| | GGPSVFLFPPKPKDTLMISRTPEVTCVVVDVSHED | |
| | PEVKFNWYVDGVEVHNAKTKPREEQYNSTYRVV | |
| | SVLTVLHQDWLNGKEYKCKVSNKALPAPIEKTIS | |
| | KAKGQPREPQVYTLPPSREEMTKNQVSLTCLVKG | |
| | FYPSDIAVEWESNGQPENNYKTTPPVLDSDGSFFL | |
| | YSKLTVDKSRWQQGNVFSCSVMHEALHNHYTQK | |
| | SLSLSPG, wherein X is glutamine (Q) or pyroglutamate | |
| | (pE) | |
| full-length heavy chain | XVQLVQSGAEVKKPGASVKVSCKASGYTFTSYA | 99 |
| (with C-terminal lysine) - | MHWVRQAPGQRLEWMGWINAGNGNTKYSQKFQ | |
| BA100 | GRVTITRDTSASTAYMELSSLRSEDTAVYYCARN | |
| | WGLSYGLDVWGQGTTVTVSSASTKGPSVFPLAPS | |
| | SKSTSGGTAALGCLVKDYFPEPVTVSWNSGALTS | |
| | GVHTFPAVLQSSGLYSLSSVVTVPSSSLGTQTYICN | |
| | VNHKPSNTKVDKRVEPKSCDKTHTCPPCPAPELL | |
| | GGPSVFLFPPKPKDTLMISRTPEVTCVVVDVSHED | |
| | PEVKFNWYVDGVEVHNAKTKPREEQYNSTYRVV | |
| | SVLTVLHQDWLNGKEYKCKVSNKALPAPIEKTIS | |
| | KAKGQPREPQVYTLPPSREEMTKNQVSLTCLVKG | |
| | FYPSDIAVEWESNGQPENNYKTTPPVLDSDGSFFL | |
| | YSKLTVDKSRWQQGNVFSCSVMHEALHNHYTQK | |
| | SLSLSPGK, wherein X is glutamine (Q) or | |
| | pyroglutamate (pE) | |
| full-length heavy chain | XVQLVQSGAEVKKPGASVKVSCKASGYTFTSYA | 167 |
| (without C-terminal | MHWVRQAPGQRLEWMGWINAGNGNTKYSQKFQ | |
| lysine) - BA100 | GRVTITRDTSASTAYMELSSLRSEDTAVYYCARN | |
| | WGLSYGLDVWGQGTTVTVSSASTKGPSVFPLAPS | |
| | SKSTSGGTAALGCLVKDYFPEPVTVSWNSGALTS | |
| | GVHTFPAVLQSSGLYSLSSVVTVPSSSLGTQTYICN | |
| | VNHKPSNTKVDKRVEPKSCDKTHTCPPCPAPELL | |
| | GGPSVFLFPPKPKDTLMISRTPEVTCVVVDVSHED | |
| | PEVKFNWYVDGVEVHNAKTKPREEQYNSTYRVV | |
| | SVLTVLHQDWLNGKEYKCKVSNKALPAPIEKTIS | |
| | KAKGQPREPQVYTLPPSREEMTKNQVSLTCLVKG | |
| | FYPSDIAVEWESNGQPENNYKTTPPVLDSDGSFFL | |
| | YSKLTVDKSRWQQGNVFSCSVMHEALHNHYTQK | |
| | SLSLSPG, wherein X is glutamine (Q) or pyroglutamate | |
| | (pE) | |
| full-length heavy chain | XVQLVQSGAEVKKPGSSVKVSCKASGGTFSSYAIS | 100 |
| (with C-terminal lysine) - | WVRQAPGQGLEWMGGIIPIFGTANYAQKFQGRVT | |
| BA101, BA102, BA103, | ITADKSTSTAYMELSSLRSEDTAVYYCARGYDSRP | |
| BA104, BA105, BA106 | LDVWGQGTLVTVSSASTKGPSVFPLAPSSKSTSGG | |
| | TAALGCLVKDYFPEPVTVSWNSGALTSGVHTFPA | |
| | VLQSSGLYSLSSVVTVPSSSLGTQTYICNVNHKPS | |
| | NTKVDKRVEPKSCDKTHTCPPCPAPELLGGPSVFL | |
| | FPPKPKDTLMISRTPEVTCVVVDVSHEDPEVKFN | |
| | WYVDGVEVHNAKTKPREEQYNSTYRVVSVLTVL | |
| | HQDWLNGKEYKCKVSNKALPAPIEKTISKAKGQP | |
| | REPQVYTLPPSREEMTKNQVSLTCLVKGFYPSDIA | |
| | VEWESNGQPENNYKTTPPVLDSDGSFFLYSKLTV | |
| | DKSRWQQGNVFSCSVMHEALHNHYTQKSLSLSP | |
| | GK, wherein X is glutamine (Q) or pyroglutamate (pE) | |
| full-length heavy chain | XVQ LVQ SGAEVKKPGSSVKVSCKASGGTFSSYAIS | 168 |
| (without C-terminal | WVRQAPGQGLEWMGGIIPIFGTANYAQKFQGRVT | |
| lysine) - BA101, BA102, | ITADKSTSTAYMELSSLRSEDTAVYYCARGYDSRP | |
| BA103, BA104, BA105, | LDVWGQGTLVTVSSASTKGPSVFPLAPSSKSTSGG | |
| BA106 | TAALGCLVKDYFPEPVTVSWNSGALTSGVHTFPA | |
| | VLQSSGLYSLSSVVTVPSSSLGTQTYICNVNHKPS | |
| | NTKVDKRVEPKSCDKTHTCPPCPAPELLGGPSVFL | |
| | FPPKPKDTLMISRTPEVTCVVVDVSHEDPEVKFN | |
| | WYVDGVEVHNAKTKPREEQYNSTYRVVSVLTVL | |
| | HQDWLNGKEYKCKVSNKALPAPIEKTISKAKGQP | |
| | REPQVYTLPPSREEMTKNQVSLTCLVKGFYPSDIA | |
| | VEWESNGQPENNYKTTPPVLDSDGSFFLYSKLTV | |
| | DKSRWQQGNVFSCSVMHEALHNHYTQKSLSLSP | |
| | G, wherein X is glutamine (Q) or pyroglutamate (pE) | |
| full-length heavy chain | XVQLVQSGAEVKKPGSSVKVSCKASGGTFSSYAIS | 101 |
| (with C-terminal lysine) - | WVRQAPGQGLEWMGGIIPIFGTANYAQKFQGRVT | |
| BA107 | ITADKSTSTAYMELSSLRSEDTAVYYCARGYDSRP | |
| | LDYWGQGTLVTVSSASTKGPSVFPLAPSSKSTSGG | |
| | TAALGCLVKDYFPEPVTVSWNSGALTSGVHTFPA | |
| | VLQSSGLYSLSSVVTVPSSSLGTQTYICNVNHKPS | |
| | NTKVDKRVEPKSCDKTHTCPPCPAPELLGGPSVFL | |
| | FPPKPKDTLMISRTPEVTCVVVDVSHEDPEVKFN | |
| | WYVDGVEVHNAKTKPREEQYNSTYRVVSVLTVL | |
| | HQDWLNGKEYKCKVSNKALPAPIEKTISKAKGQP | |
| | REPQVYTLPPSREEMTKNQVSLTCLVKGFYPSDIA | |
| | VEWESNGQPENNYKTTPPVLDSDGSFFLYSKLTV | |
| | DKSRWQQGNVFSCSVMHEALHNHYTQKSLSLSP | |
| | GK, wherein X is glutamine (Q) or pyroglutamate (pE) | |
| full-length heavy chain | XVQLVQSGAEVKKPGSSVKVSCKASGGTFSSYAIS | 169 |
| (without C-terminal | WVRQAPGQGLEWMGGIIPIFGTANYAQKFQGRVT | |
| lysine) - BA107 | ITADKSTSTAYMELSSLRSEDTAVYYCARGYDSRP | |
| | LDYWGQGTLVTVSSASTKGPSVFPLAPSSKSTSGG | |
| | TAALGCLVKDYFPEPVTVSWNSGALTSGVHTFPA | |
| | VLQSSGLYSLSSVVTVPSSSLGTQTYICNVNHKPS | |
| | NTKVDKRVEPKSCDKTHTCPPCPAPELLGGPSVFL | |
| | FPPKPKDTLMISRTPEVTCVVVDVSHEDPEVKFN | |
| | WYVDGVEVHNAKTKPREEQYNSTYRVVSVLTVL | |
| | HQDWLNGKEYKCKVSNKALPAPIEKTISKAKGQP | |
| | REPQVYTLPPSREEMTKNQVSLTCLVKGFYPSDIA | |
| | VEWESNGQPENNYKTTPPVLDSDGSFFLYSKLTV | |
| | DKSRWQQGNVFSCSVMHEALHNHYTQKSLSLSP | |
| | G, wherein X is glutamine (Q) or pyroglutamate (pE) | |
| full-length light chain - | DIQMTQSPSSLSASVGDRVTITCRASQSISSYLNWY | 102 |
| BA072, BA083, BA084, | QQKPGKAPKLLIYAASSLQSGVPSRFSGSGSGTDF | |
| BA089, BA090, BA091, | TLTISSLQPEDFATYYCQQSYSTPALTFGGGTKVDI | |
| BA092, BA093 | KRTVAAPSVFIFPPSDEQLKSGTASVVCLLNNFYP | |
| | REAKVQWKVDNALQSGNSQESVTEQDSKDSTYS | |
| | LSSTLTLSKADYEKHKVYACEVTHQGLSSPVTKSF | |
| | NRGEC | |
| full-length light chain - | DIQMTQSPSSLSASVGDRVTITCRASQSISSYLNWY | 103 |
| BA074, BA073, BA081, | QQKPGKAPKLLIYSASSLQSGVPSRFSGSGSGTDFT | |
| BA080, BA075, BA079, | LTISSLQPEDFATYYCQQSYSTPALTFGGGTKVDIK | |
| BA078, BA088 | RTVAAPSVFIFPPSDEQLKSGTASVVCLLNNFYPRE | |
| | AKVQWKVDNALQSGNSQESVTEQDSKDSTYSLSS | |
| | TLTLSKADYEKHKVYACEVTHQGLSSPVTKSFNR | |
| | GEC | |
| full-length light chain - | DIQMTQSPSSLSASVGDRVTITCRASQSISSYLNWY | 104 |
| BA077, BA076 | QQKPGKAPKLLIYSESSLQSGVPSRFSGSGSGTDFT | |
| | LTISSLQPEDFATYYCQQSYSTPALTFGGGTKVDIK | |
| | RTVAAPSVFIFPPSDEQLKSGTASVVCLLNNFYPRE | |
| | AKVQWKVDNALQSGNSQESVTEQDSKDSTYSLSS | |
| | TLTLSKADYEKHKVYACEVTHQGLSSPVTKSFNR | |
| | GEC | |
| full-length light chain - | DIQMTQSPSSLSASVGDRVTITCRASQSISSYLNWY | 105 |
| BA082 | QQKPGKAPKLLIYSSSSLQSGVPSRFSGSGSGTDFT | |
| | LTISSLQPEDFATYYCQQSYSTPALTFGGGTKVDIK | |
| | RTVAAPSVFIFPPSDEQLKSGTASVVCLLNNFYPRE | |
| | AKVQWKVDNALQSGNSQESVTEQDSKDSTYSLSS | |
| | TLTLSKADYEKHKVYACEVTHQGLSSPVTKSFNR | |
| | GEC | |
| full-length light chain - | DIQMTQSPSSLSASVGDRVTITCRASQSISPYLNWY | 106 |
| BA085 | QQKPGKAPKLLIYSASSLQSGVPSRFSGSGSGTDFT | |
| | LTISSLQPEDFATYYCQQSYSTPALTFGGGTKVDIK | |
| | RTVAAPSVFIFPPSDEQLKSGTASVVCLLNNFYPRE | |
| | AKVQWKVDNALQSGNSQESVTEQDSKDSTYSLSS | |
| | TLTLSKADYEKHKVYACEVTHQGLSSPVTKSFNR | |
| | GEC | |
| full-length light chain - | DIQMTQSPSSLSASVGDRVTITCRASQSISWYLNW | 107 |
| BA086 | YQQKPGKAPKLLIYSASSLQSGVPSRFSGSGSGTD | |
| | FTLTISSLQPEDFATYYCQQSYSTPALTFGGGTKV | |
| | DIKRTVAAPSVFIFPPSDEQLKSGTASVVCLLNNFY | |
| | PREAKVQWKVDNALQSGNSQESVTEQDSKDSTY | |
| | SLSSTLTLSKADYEKHKVYACEVTHQGLSSPVTKS | |
| | FNRGEC | |
| full-length light chain - | DIQMTQSPSSLSASVGDRVTITCRASQSILSYLNW | 108 |
| BA087 | YQQKPGKAPKLLIYSASSLQSGVPSRFSGSGSGTD | |
| | FTLTISSLQPEDFATYYCQQSYSTPALTFGGGTKV | |
| | DIKRTVAAPSVFIFPPSDEQLKSGTASVVCLLNNFY | |
| | PREAKVQWKVDNALQSGNSQESVTEQDSKDSTY | |
| | SLSSTLTLSKADYEKHKVYACEVTHQGLSSPVTKS | |
| | FNRGEC | |
| full-length light chain - | DIQMTQSPSSLSASVGDRVTITCRASQSITSYLNW | 109 |
| BA094 | YQQKPGKAPKLLIYAASSLQSGVPSRFSGSGSGTD | |
| | FTLTISSLQPEDFATYYCQQAYSTPALSFGGGTKV | |
| | DIKRTVAAPSVFIFPPSDEQLKSGTASVVCLLNNFY | |
| | PREAKVQWKVDNALQSGNSQESVTEQDSKDSTY | |
| | SLSSTLTLSKADYEKHKVYACEVTHQGLSSPVTKS | |
| | FNRGEC | |
| full-length light chain - | SYELTQPLSVSVALGQTASITCGGNNIGSKIVHWY | 110 |
| BA101, BA107 | QQKSGQAPVLVVSDDRDRPSGIPERFSGSNSGNTA | |
| | TLTINTVEAGDEADYYCQVWDINVHHVIFGGGTK | |
| | VTVLGQPKAAPSVTLFPPSSEELQANKATLVCLIS | |
| | DFYPGAVTVAWKADSSPVKAGVETTTPSKQSNN | |
| | KYAASSYLSLTPEQWKSHRSYSCQVTHEGSTVEK | |
| | TVAPTECS | |
| full-length light chain - | SYELTQPLSVSVALGQTASITCGGNNIGSKIVHWY | 111 |
| BA102 | QQKPGQAPVLVVSDDRDRPSGIPERFSGSNSGNTA | |
| | TLTISRVEAGDEADYYCQVWDINVHHVIFGGGTK | |
| | LTVLGQPKAAPSVTLFPPSSEELQANKATLVCLISD | |
| | FYPGAVTVAWKADSSPVKAGVETTTPSKQSNNK | |
| | YAASSYLSLTPEQWKSHRSYSCQVTHEGSTVEKT | |
| | VAPTECS | |
| full-length light chain - | SYELTQPLSVSVALGQTASITCGGNNIGSKIVHWY | 112 |
| BA103 | QQKSGQAPVLVIYDDRDRPSGIPERFSGSNSGNTA | |
| | TLTINTVEAGDEADYYCQVWDINVHHVIFGGGTK | |
| | LTVLGQPKAAPSVTLFPPSSEELQANKATLVCLISD | |
| | FYPGAVTVAWKADSSPVKAGVETTTPSKQSNNK | |
| | YAASSYLSLTPEQWKSHRSYSCQVTHEGSTVEKT | |
| | VAPTECS | |
| full-length light chain - | SYELTQPLSVSVALGQTASITCGGNNIGSKIVHWY | 113 |
| BA104 | QQKPGQAPVLVVSDDRDRPSGIPERFSGSNSGNTA | |
| | TLTINTVEAGDEADYYCQVWDINVHHVIFGGGTK | |
| | LTVLGQPKAAPSVTLFPPSSEELQANKATLVCLISD | |
| | FYPGAVTVAWKADSSPVKAGVETTTPSKQSNNK | |
| | YAASSYLSLTPEQWKSHRSYSCQVTHEGSTVEKT | |
| | VAPTECS | |
| full-length light chain - | SYELTQPLSVSVALGQTASITCGGNNIGSKIVHWY | 114 |
| BA105 | QQKSGQAPVLVVSDDRDRPSGIPERFSGSNSGNTA | |
| | TLTISRVEAGDEADYYCQVWDINVHHVIFGGGTK | |
| | LTVLGQPKAAPSVTLFPPSSEELQANKATLVCLISD | |
| | FYPGAVTVAWKADSSPVKAGVETTTPSKQSNNK | |
| | YAASSYLSLTPEQWKSHRSYSCQVTHEGSTVEKT | |
| | VAPTECS | |
| full-length light chain - | SYELTQPLSVSVALGQTASITCGGNNIGSKIVHWY | 115 |
| BA106 | QQKSGQAPVLVVSDDRDRPSGIPERFSGSNSGNTA | |
| | TLTISRAQAGDEADYYCQVWDINVHHVIFGGGTK | |
| | LTVLGQPKAAPSVTLFPPSSEELQANKATLVCLISD | |
| | FYPGAVTVAWKADSSPVKAGVETTTPSKQSNNK | |
| | YAASSYLSLTPEQWKSHRSYSCQVTHEGSTVEKT | |
| | VAPTECS | |
| IgG1 N297A variant full- | XVQLVQSGAEVKKPGASVKVSCKASGYTFTSYA | 116 |
| length heavy chain (with | MHWVRQAPGQRLEWMGWINAGNGNTKYSQKFQ | |
| C-terminal lysine) - | GRVTITRDTSTSTAYMELRSLRSDDTAMYYCARN | |
| BA072 | WGMSYGMDVWGQGTMVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYASTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPGK, wherein X is glutamine (Q) or | |
| | pyroglutamate (pE) | |
| IgG1 N297A variant full- | XVQLVQSGAEVKKPGASVKVSCKASGYTFTSYA | 170 |
| length heavy chain | MHWVRQAPGQRLEWMGWINAGNGNTKYSQKFQ | |
| (without C-terminal | GRVTITRDTSTSTAYMELRSLRSDDTAMYYCARN | |
| lysine) - BA072 | WGMSYGMDVWGQGTMVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYASTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPG, wherein X is glutamine (Q) or | |
| | pyroglutamate (pE) | |
| IgG1 N297A variant full- | XVQLVQSGAEVKKPGSSVKVSCKASGGTFSSYAIS | 117 |
| length heavy chain (with | WVRQAPGQGLEWMGGIIPIFGTANYAQKFQGRVT | |
| C-terminal lysine) - | ITADKSTSTAYMELSSLRSEDTAVYYCARGYD SRP | |
| BA101 | LDVWGQGTLVTVSSASTKGPSVFPLAPSSKSTSGG | |
| | TAALGCLVKDYFPEPVTVSWNSGALTSGVHTFPA | |
| | VLQSSGLYSLSSVVTVPSSSLGTQTYICNVNHKPS | |
| | NTKVDKRVEPKSCDKTHTCPPCPAPELLGGPSVFL | |
| | FPPKPKDTLMISRTPEVTCVVVDVSHEDPEVKFN | |
| | WYVDGVEVHNAKTKPREEQYASTYRVVSVLTVL | |
| | HQDWLNGKEYKCKVSNKALPAPIEKTISKAKGQP | |
| | REPQVYTLPPSREEMTKNQVSLTCLVKGFYPSDIA | |
| | VEWESNGQPENNYKTTPPVLDSDGSFFLYSKLTV | |
| | DKSRWQQGNVFSCSVMHEALHNHYTQKSLSLSP | |
| | GK, wherein X is glutamine (Q) or pyroglutamate (pE) | |
| IgG1 N297A variant full- | XVQLVQSGAEVKKPGSSVKVSCKASGGTFSSYAIS | 171 |
| length heavy chain | WVRQAPGQGLEWMGGIIPIFGTANYAQKFQGRVT | |
| (without C-terminal | ITADKSTSTAYMELSSLRSEDTAVYYCARGYD SRP | |
| lysine) - BA101 | LDVWGQGTLVTVSSASTKGPSVFPLAPSSKSTSGG | |
| | TAALGCLVKDYFPEPVTVSWNSGALTSGVHTFPA | |
| | VLQSSGLYSLSSVVTVPSSSLGTQTYICNVNHKPS | |
| | NTKVDKRVEPKSCDKTHTCPPCPAPELLGGPSVFL | |
| | FPPKPKDTLMISRTPEVTCVVVDVSHEDPEVKFN | |
| | WYVDGVEVHNAKTKPREEQYASTYRVVSVLTVL | |
| | HQDWLNGKEYKCKVSNKALPAPIEKTISKAKGQP | |
| | REPQVYTLPPSREEMTKNQVSLTCLVKGFYPSDIA | |
| | VEWESNGQPENNYKTTPPVLDSDGSFFLYSKLTV | |
| | DKSRWQQGNVFSCSVMHEALHNHYTQKSLSLSP | |
| | G, wherein X is glutamine (Q) or pyroglutamate (pE) | |
| IgG1 | XVQLVQSGAEVKKPGASVKVSCKASGYTFTSYA | 118 |
| S239D/A330L/I332E | MHWVRQAPGQRLEWMGWINAGNGNTKYSQKFQ | |
| variant full-length heavy | GRVTITRDTSTSTAYMELRSLRSDDTAMYYCARN | |
| chain (with C-terminal | WGMSYGMDVWGQGTMVTVSSASTKGPSVFPLAP | |
| lysine) - BA072 | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPDVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPLPEEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPGK, wherein X is glutamine (Q) or | |
| | pyroglutamate (pE) | |
| IgG1 | XVQLVQSGAEVKKPGASVKVSCKASGYTFTSYA | 172 |
| S239D/A330L/I332E | MHWVRQAPGQRLEWMGWINAGNGNTKYSQKFQ | |
| variant full-length heavy | GRVTITRDTSTSTAYMELRSLRSDDTAMYYCARN | |
| chain (without C- | WGMSYGMDVWGQGTMVTVSSASTKGPSVFPLAP | |
| terminal lysine) - BA072 | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPDVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPLPEEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPG, wherein X is glutamine (Q) or | |
| | pyroglutamate (pE) | |
| IgG1 | XVQLVQSGAEVKKPGSSVKVSCKASGGTFSSYAIS | 119 |
| S239D/A330L/I332E | WVRQAPGQGLEWMGGIIPIFGTANYAQKFQGRVT | |
| variant full-length heavy | ITADKSTSTAYMELSSLRSEDTAVYYCARGYDSRP | |
| chain (with C-terminal | LDVWGQGTLVTVSSASTKGPSVFPLAPSSKSTSGG | |
| lysine) - BA101 | TAALGCLVKDYFPEPVTVSWNSGALTSGVHTFPA | |
| | VLQSSGLYSLSSVVTVPSSSLGTQTYICNVNHKPS | |
| | NTKVDKRVEPKSCDKTHTCPPCPAPELLGGPDVFL | |
| | FPPKPKDTLMISRTPEVTCVVVDVSHEDPEVKFN | |
| | WYVDGVEVHNAKTKPREEQYNSTYRVVSVLTVL | |
| | HQDWLNGKEYKCKVSNKALPLPEEKTISKAKGQP | |
| | REPQVYTLPPSREEMTKNQVSLTCLVKGFYPSDIA | |
| | VEWESNGQPENNYKTTPPVLDSDGSFFLYSKLTV | |
| | DKSRWQQGNVFSCSVMHEALHNHYTQKSLSLSP | |
| | GK, wherein X is glutamine (Q) or pyroglutamate (pE) | |
| IgG1 | XVQLVQSGAEVKKPGSSVKVSCKASGGTFSSYAIS | 173 |
| S239D/A330L/I332E | WVRQAPGQGLEWMGGIIPIFGTANYAQKFQGRVT | |
| variant full-length heavy | ITADKSTSTAYMELSSLRSEDTAVYYCARGYDSRP | |
| chain (without C- | LDVWGQGTLVTVSSASTKGPSVFPLAPSSKSTSGG | |
| terminal lysine) - BA101 | TAALGCLVKDYFPEPVTVSWNSGALTSGVHTFPA | |
| | VLQSSGLYSLSSVVTVPSSSLGTQTYICNVNHKPS | |
| | NTKVDKRVEPKSCDKTHTCPPCPAPELLGGPDVFL | |
| | FPPKPKDTLMISRTPEVTCVVVDVSHEDPEVKFN | |
| | WYVDGVEVHNAKTKPREEQYNSTYRVVSVLTVL | |
| | HQDWLNGKEYKCKVSNKALPLPEEKTISKAKGQP | |
| | REPQVYTLPPSREEMTKNQVSLTCLVKGFYPSDIA | |
| | VEWESNGQPENNYKTTPPVLDSDGSFFLYSKLTV | |
| | DKSRWQQGNVFSCSVMHEALHNHYTQKSLSLSP | |
| | G, wherein X is glutamine (Q) or pyroglutamate (pE) | |
| IgG1 S267E L328F | XVQLVQSGAEVKKPGASVKVSCKASGYTFTSYA | 120 |
| variant full-length heavy | MHWVRQAPGQRLEWMGWINAGNGNTKYSQKFQ | |
| chain (with C-terminal | GRVTITRDTSTSTAYMELRSLRSDDTAMYYCARN | |
| lysine) - BA072 | WGMSYGMDVWGQGTMVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVEHE | |
| | DPEVKFNWYVDGVEVHNAKTKPREEQYNSTYRV | |
| | VSVLTVLHQDWLNGKEYKCKVSNKAFPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPGK, wherein X is glutamine (Q) or | |
| | pyroglutamate (pE) | |
| IgG1 S267E L328F | XVQLVQSGAEVKKPGASVKVSCKASGYTFTSYA | 174 |
| variant full-length heavy | MHWVRQAPGQRLEWMGWINAGNGNTKYSQKFQ | |
| chain (without C- | GRVTITRDTSTSTAYMELRSLRSDDTAMYYCARN | |
| terminal lysine) - BA072 | WGMSYGMDVWGQGTMVTVSSASTKGPSVFPLAP | |
| | SSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVEHE | |
| | DPEVKFNWYVDGVEVHNAKTKPREEQYNSTYRV | |
| | VSVLTVLHQDWLNGKEYKCKVSNKAFPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPG, wherein X is glutamine (Q) or | |
| | pyroglutamate (pE) | |
| heavy chain constant | ASTKGPSVFPLAPSSKSTSGGTAALGCLVKDYFPE | 121 |
| region (with C-terminal | PVTVSWNSGALTSGVHTFPAVLQSSGLYSLSSVVT | |
| lysine) - BA072, BA101 | VPSSSLGTQTYICNVNHKPSNTKVDKRVEPKSCDK | |
| | THTCPPCPAPELLGGPSVFLFPPKPKDTLMISRTPE | |
| | VTCVVVDVSHEDPEVKFNWYVDGVEVHNAKTKP | |
| | REEQYNSTYRVVSVLTVLHQDWLNGKEYKCKVS | |
| | NKALPAPIEKTISKAKGQPREPQVYTLPPSREEMT | |
| | KNQVSLTCLVKGFYPSDIAVEWESNGQPENNYKT | |
| | TPPVLDSDGSFFLYSKLTVDKSRWQQGNVFSCSV | |
| | MHEALHNHYTQKSLSLSPGK | |
| heavy chain constant | ASTKGPSVFPLAPSSKSTSGGTAALGCLVKDYFPE | 175 |
| region (without C- | PVTVSWNSGALTSGVHTFPAVLQSSGLYSLSSVVT | |
| terminal lysine) - BA072, | VPSSSLGTQTYICNVNHKPSNTKVDKRVEPKSCDK | |
| BA101 | THTCPPCPAPELLGGPSVFLFPPKPKDTLMISRTPE | |
| | VTCVVVDVSHEDPEVKFNWYVDGVEVHNAKTKP | |
| | REEQYNSTYRVVSVLTVLHQDWLNGKEYKCKVS | |
| | NKALPAPIEKTISKAKGQPREPQVYTLPPSREEMT | |
| | KNQVSLTCLVKGFYPSDIAVEWESNGQPENNYKT | |
| | TPPVLDSDGSFFLYSKLTVDKSRWQQGNVFSCSV | |
| | MHEALHNHYTQKSLSLSPG | |
| light chain constant | RTVAAPSVFIFPPSDEQLKSGTASVVCLLNNFYPRE | 122 |
| region - BA072 | AKVQWKVDNALQSGNSQESVTEQDSKDSTYSLSS | |
| | TLTLSKADYEKHKVYACEVTHQGLSSPVTKSFNR | |
| | GEC | |
| light chain constant | GQPKAAPSVTLFPPSSEELQANKATLVCLISDFYPG | 123 |
| region - BA101 | AVTVAWKADSSPVKAGVETTTPSKQSNNKYAAS | |
| | SYLSLTPEQWKSHRSYSCQVTHEGSTVEKTVAPTE | |
| | CS | |
| IgG1 N297A variant | ASTKGPSVFPLAPSSKSTSGGTAALGCLVKDYFPE | 124 |
| constant region (with C- | PVTVSWNSGALTSGVHTFPAVLQSSGLYSLSSVVT | |
| terminal lysine) - BA072 | VPSSSLGTQTYICNVNHKPSNTKVDKRVEPKSCDK | |
| | THTCPPCPAPELLGGPSVFLFPPKPKDTLMISRTPE | |
| | VTCVVVDVSHEDPEVKFNWYVDGVEVHNAKTKP | |
| | REEQYASTYRVVSVLTVLHQDWLNGKEYKCKVS | |
| | NKALPAPIEKTISKAKGQPREPQVYTLPPSREEMT | |
| | KNQVSLTCLVKGFYPSDIAVEWESNGQPENNYKT | |
| | TPPVLDSDGSFFLYSKLTVDKSRWQQGNVFSCSV | |
| | MHEALHNHYTQKSLSLSPGK | |
| IgG1 N297A variant | ASTKGPSVFPLAPSSKSTSGGTAALGCLVKDYFPE | 176 |
| constant region (without | PVTVSWNSGALTSGVHTFPAVLQSSGLYSLSSVVT | |
| C-terminal lysine) - | VPSSSLGTQTYICNVNHKPSNTKVDKRVEPKSCDK | |
| BA072 | THTCPPCPAPELLGGPSVFLFPPKPKDTLMISRTPE | |
| | VTCVVVDVSHEDPEVKFNWYVDGVEVHNAKTKP | |
| | REEQYASTYRVVSVLTVLHQDWLNGKEYKCKVS | |
| | NKALPAPIEKTISKAKGQPREPQVYTLPPSREEMT | |
| | KNQVSLTCLVKGFYPSDIAVEWESNGQPENNYKT | |
| | TPPVLDSDGSFFLYSKLTVDKSRWQQGNVFSCSV | |
| | MHEALHNHYTQKSLSLSPG | |
| IgG1 | ASTKGPSVFPLAPSSKSTSGGTAALGCLVKDYFPE | 125 |
| 5239D/A330L/I332E | PVTVSWNSGALTSGVHTFPAVLQSSGLYSLSSVVT | |
| variant constant region | VPSSSLGTQTYICNVNHKPSNTKVDKRVEPKSCDK | |
| (with C-terminal lysine) - | THTCPPCPAPELLGGPDVFLFPPKPKDTLMISRTPE | |
| BA072 | VTCVVVDVSHEDPEVKFNWYVDGVEVHNAKTKP | |
| | REEQYNSTYRVVSVLTVLHQDWLNGKEYKCKVS | |
| | NKALPLPEEKTISKAKGQPREPQVYTLPPSREEMT | |
| | KNQVSLTCLVKGFYPSDIAVEWESNGQPENNYKT | |
| | TPPVLDSDGSFFLYSKLTVDKSRWQQGNVFSCSV | |
| | MHEALHNHYTQKSLSLSPGK | |
| IgG1 | ASTKGPSVFPLAPSSKSTSGGTAALGCLVKDYFPE | 177 |
| 5239D/A330L/I332E | PVTVSWNSGALTSGVHTFPAVLQSSGLYSLSSVVT | |
| variant constant region | VPSSSLGTQTYICNVNHKPSNTKVDKRVEPKSCDK | |
| (without C-terminal | THTCPPCPAPELLGGPDVFLFPPKPKDTLMISRTPE | |
| lysine) - BA072 | VTCVVVDVSHEDPEVKFNWYVDGVEVHNAKTKP | |
| | REEQYNSTYRVVSVLTVLHQDWLNGKEYKCKVS | |
| | NKALPLPEEKTISKAKGQPREPQVYTLPPSREEMT | |
| | KNQVSLTCLVKGFYPSDIAVEWESNGQPENNYKT | |
| | TPPVLDSDGSFFLYSKLTVDKSRWQQGNVFSCSV | |
| | MHEALHNHYTQKSLSLSPG | |
| IgG1 S267E L328F | ASTKGPSVFPLAPSSKSTSGGTAALGCLVKDYFPE | 126 |
| variant constant region | PVTVSWNSGALTSGVHTFPAVLQSSGLYSLSSVVT | |
| (with C-terminal lysine) - | VPSSSLGTQTYICNVNHKPSNTKVDKRVEPKSCDK | |
| BA072 | THTCPPCPAPELLGGPSVFLFPPKPKDTLMISRTPE | |
| | VTCVVVDVEHEDPEVKFNWYVDGVEVHNAKTKP | |
| | REEQYNSTYRVVSVLTVLHQDWLNGKEYKCKVS | |
| | NKAFPAPIEKTISKAKGQPREPQVYTLPPSREEMT | |
| | KNQVSLTCLVKGFYPSDIAVEWESNGQPENNYKT | |
| | TPPVLDSDGSFFLYSKLTVDKSRWQQGNVFSCSV | |
| | MHEALHNHYTQKSLSLSPGK | |
| IgG1 S267E L328F | ASTKGPSVFPLAPSSKSTSGGTAALGCLVKDYFPE | 178 |
| variant constant region | PVTVSWNSGALTSGVHTFPAVLQSSGLYSLSSVVT | |
| (without C-terminal | VPSSSLGTQTYICNVNHKPSNTKVDKRVEPKSCDK | |
| lysine) - BA072 | THTCPPCPAPELLGGPSVFLFPPKPKDTLMISRTPE | |
| | VTCVVVDVEHEDPEVKFNWYVDGVEVHNAKTKP | |
| | REEQYNSTYRVVSVLTVLHQDWLNGKEYKCKVS | |
| | NKAFPAPIEKTISKAKGQPREPQVYTLPPSREEMT | |
| | KNQVSLTCLVKGFYPSDIAVEWESNGQPENNYKT | |
| | TPPVLDSDGSFFLYSKLTVDKSRWQQGNVFSCSV | |
| | MHEALHNHYTQKSLSLSPG | |

1.-71. (canceled)

72. An isolated polynucleotide encoding a heavy chain variable region (VH) and/or a light chain variable region (VL), of an antibody that specifically binds to human CD96, the antibody comprising a VH comprising complementarity determining regions (CDRs) CDRH1, CDRH2, and CDRH3, and a VL comprising CDRs CDRL1, CDRL2, and CDRL3, wherein:
(a) CDRH1 comprises the amino acid sequence of X₁YX₂X₃X₄ (SEQ ID NO: 135), wherein
X₁ is Q or S;
X₂ is A or S;
X₃ is M or I; and
X₄ is H or S;
(b) CDRH2 comprises the amino acid sequence of X₁IX₂X₃X₄X₅X₆X₇X₈X₉YX₁₀QKFQG (SEQ ID NO: 137), wherein
X₁ is W or G;
X₂ is N or I;
X₃ is A, E, V, or P;
X₄ is V, G, W, or I;
X₅ is S, Y, T, N, or F;
X₆ is G or W;
X₇ is D, Y, N, or T;
X₈ is T or A;
X₉ is K or N; and
X₁₀ is S or A;
(c) CDRH3 comprises the amino acid sequence of NWGX₁SYGX₂DV (SEQ ID NO: 180), SEQ ID NO: 19, or SEQ ID NO: 20, wherein
X₁ is M or L; and
X₂ is M or L;
(d) CDRL1 comprises the amino acid sequence of RASQSIX₁X₂YLN (SEQ ID NO: 139) or SEQ ID NO: 26, wherein
X₁ is S, T, or L; and
X₂ is S, P, or W;
(e) CDRL2 comprises the amino acid sequence of X₁X₂SSLQS (SEQ ID NO: 141) or SEQ ID NO: 32, wherein
X₁ is S or A; and
X₂ is A, S, or E; and/or
(f) CDRL3 comprises the amino acid sequence of QQX₁YSTPALX₂ (SEQ ID NO: 143) or SEQ ID NO: 35, wherein
X₁ is S or A; and
X₂ is T or S.

73. The isolated polynucleotide of claim 72, wherein:
(a) CDRH1 comprises the amino acid sequence of X₁YX₂MH (SEQ ID NO: 136), wherein
X₁ is Q or S; and
X₂ is A or S;
(b) CDRH2 comprises the amino acid sequence of WINX₁X₂X₃X₄X₅TKYSQKFQG (SEQ ID NO: 138), wherein
X₁ is A, V, or E;
X₂ is V, W, or G;
X₃ is S, Y, T, or N;
X₄ is G or W; and
X₅ is D, N, Y, or T;
(c) CDRH3 comprises the amino acid sequence of NWGX₁SYGX₂DV (SEQ ID NO: 180), wherein
X₁ is M or L; and
X₂ is M or L;
(d) CDRL1 comprises the amino acid sequence of RASQSIX₁X₂YLN (SEQ ID NO: 139), wherein
X₁ is S, T, or L; and
X₂ is S, P, or W;
(e) CDRL2 comprises the amino acid sequence of X₁X₂SSLQS (SEQ ID NO: 141), wherein
X₁ is S or A; and
X₂ is A, S, or E; and/or
(f) CDRL3 comprises the amino acid sequence of SEQ ID NO: 33 or SEQ ID NO: 34.

74. The isolated polynucleotide of claim 72, wherein:
(a) (i) CDRH1 comprises the amino acid sequence of SEQ ID NO: 4;
(ii) CDRH2 comprises the amino acid sequence of SEQ ID NO: 17;
(iii) CDRH3 comprises the amino acid sequence of SEQ ID NO: 19 or 20;
(iv) CDRL1 comprises the amino acid sequence of SEQ ID NO: 26;
(v) CDRL2 comprises the amino acid sequence of SEQ ID NO: 32; and/or
(vi) CDRL3 comprises the amino acid sequence of SEQ ID NO: 35;
(b) CDRH1, CDRH2, and CDRH3 comprise the amino acid sequences of SEQ ID NOs: 1, 5, and 18; 2, 6, and 18; 2, 8, and 18; 2, 9, and 18; 2, 10, and 18; 1, 7, and 18; 2, 11, and 18; 1, 12, and 18; 1, 13, and 18; 1, 14, and 18; 3, 15, and 18; 1, 16, and 18; 1, 5, and 140; 1, 5, and 142; 1, 5, and 179; 4, 17, and 19; or 4, 17, and 20, respectively;
(c) CDRL1, CDRL2, and CDRL3 comprise the amino acid sequences of SEQ ID NOs: 21, 28, and 33; 21, 29, and 33; 21, 30, and 33; 21, 31, and 33; 22, 29, and 33; 24, 29, and 33; 23, 29, and 33; 25, 28, and 34; or 26, 32, and 35, respectively; or
(d) CDRH1, CDRH2, CDRH3, CDRL1, CDRL2, and CDRL3 comprise the amino acid sequences of SEQ ID NOs: 1, 5, 18, 21, 28, and 33; 1, 5, 18, 21, 29, and 33; 1, 5, 18, 22, 29, and 33; 1, 5, 18, 23, 29, and 33; 1, 5, 18, 24, 29, and 33; 1, 5, 18, 25, 28, and 34; 1, 5, 140, 21, 28, and 33; 1, 5, 142, 21, 28, and 33; 1, 5, 179, 21, 28, and 33; 1, 7, 18, 21, 29, and 33; 1, 12, 18, 21, 28, and 33; 1, 13, 18, 21, 28, and 33; 1, 14, 18, 21, 28, and 33; 1, 16, 18, 21, 28, and 33; 2, 6, 18, 21, 29, and 33; 2, 8, 18, 21, 29, and 33; 2, 9, 18, 21, 30, and 33; 2, 10, 18, 21, 29, and 33; 2, 11, 18, 21, 31, and 33; 3, 15, 18, 21, 28, and 33; 4, 17, 19, 26, 32, and 35; or 4, 17, 20, 26, 32, and 35, respectively.

75. The isolated polynucleotide of claim 72, wherein:
(a) the antibody comprises a VH, wherein
(i) the VH comprises an amino acid sequence that is at least 75% identical to the amino acid sequence of SEQ ID NO: 36, 37, 38, 39, 40, 41, 42, 43, 44, 45, 46, 47, 48, 49, 50, 51, 52, 53, 54, 55, 56, 57, 58, 59, 60, or 61; or
(ii) the VH consists of the amino acid sequence of SEQ ID NO: 36, 37, 38, 39, 40, 41, 42, 43, 44, 45, 46, 47, 48, 49, 50, 51, 52, 53, 54, 55, 56, 57, 58, 59, 60, or 61; wherein the X in any one of SEQ ID NOs: 36, 37, 38, 39, 40, 41, 42, 43, 44, 45, 46, 47, 48, 49, 50, 51, 52, 53, 54, 55, 56, 57, 58, 59, 60, or 61 is glutamine or pyroglutamate; or
(b) the antibody comprises a VL, wherein
(i) the VL comprises an amino acid sequence that is at least 75% identical to the amino acid sequence of SEQ ID NO: 62, 63, 64, 65, 66, 67, 68, 69, 70, 71, 72, 73, 74, or 75; or
(ii) the VL consists of the amino acid sequence of SEQ ID NO: 62, 63, 64, 65, 66, 67, 68, 69, 70, 71, 72, 73, 74, or 75.

76. An isolated polynucleotide encoding an antibody that specifically binds to human CD96, the antibody comprising: a VH comprising the amino acid sequence of SEQ ID NO: 36, 37, 38, 39, 40, 41, 42, 43, 44, 45, 46, 47, 48, 49, 50, 51, 52, 53, 54, 55, 56, 57, 58, 59, 60, or 61; and/or a VL comprising the amino acid sequence of SEQ ID NO: 62, 63, 64, 65, 66, 67, 68, 69, 70, 71, 72, 73, 74, or 75,
optionally wherein the VH and VL comprise the amino acid sequences of SEQ ID NOs: 36 and 62; 37 and 62; 37 and 63; 37 and 66; 37 and 67; 37 and 68; 37 and 69; 38 and 63; 39 and 63; 40 and 63; 41 and 63; 42 and 63; 43 and 64; 44 and 64; 45 and 63; 46 and 63; 47 and 65; 48 and 62; 49 and 62; 50 and 62; 51 and 62; 52 and 62; 53 and 62; 54 and 62; 55 and 62; 56 and 62; 57 and 62; 58 and 62; 59 and 62; 60 and 70; 60 and 71; 60 and 72; 60 and 73; 60 and 74; 60 and 75; or 61 and 70, respectively,
wherein the X in any one of SEQ ID NOs: 36, 37, 38, 39, 40, 41, 42, 43, 44, 45, 46, 47, 48, 49, 50, 51, 52, 53, 54, 55, 56, 57, 58, 59, 60, or 61 is glutamine or pyroglutamate.

77. The isolated polynucleotide of claim 72, wherein the antibody specifically binds to the amino acid sequence of SEQ ID NO: 130 or 131, optionally wherein the antibody binds to the amino acid sequence of SEQ ID NO: 134.

78. An isolated polynucleotide encoding an antibody that specifically binds to the amino acid sequence of SEQ ID NO: 130 or 131, optionally wherein the antibody binds to the amino acid sequence of SEQ ID NO: 134.

79. The isolated polynucleotide of claim 72, wherein the antibody is internalized upon binding to cells expressing human CD96.

80. An isolated polynucleotide encoding an antibody that specifically binds to human CD96, wherein the antibody is internalized upon binding to cells expressing human CD96.

81. The isolated polynucleotide of claim 72, wherein the antibody comprises a heavy chain constant region selected from the group consisting of human IgG₁, IgG₂, IgG₃, IgG₄, IgA₁, and IgA₂,
optionally wherein the antibody comprises an IgG1 heavy chain constant region, optionally wherein:
(a) the amino acid sequence of the IgG₁ heavy chain constant region comprises an N297A mutation, numbered according to the EU numbering system, optionally wherein the antibody comprises a heavy chain constant region comprising the amino acid sequence of SEQ ID NO: 124 or 176;
(b) the amino acid sequence of the IgG₁ heavy chain constant region comprises S239D, A330L, and 1332E mutations, numbered according to the EU numbering system, optionally wherein the antibody comprises a heavy chain constant region comprising the amino acid sequence of SEQ ID NO: 125 or 177;
(c) the amino acid sequence of the IgG1 heavy chain constant region comprises S267E and L328F mutations, numbered according to the EU numbering system, optionally wherein the antibody comprises a heavy chain constant region comprising the amino acid sequence of SEQ ID NO: 126 or 178.

82. The isolated polynucleotide of claim 72, wherein the antibody comprises a heavy chain constant region that is a variant of a wild type heavy chain constant region, wherein the variant heavy chain constant region binds to an FcγR with higher affinity than the wild type heavy chain constant region binds to the FcγR, optionally wherein the FcγR is FcγRIIB.

83. The isolated polynucleotide of claim 72, wherein the antibody comprises a heavy chain comprising the amino acid sequence of SEQ ID NO: 76, 77, 78, 79, 80, 81, 82, 83, 84, 85, 86, 87, 88, 89, 90, 91, 92, 93, 94, 95, 96, 97, 98, 99, 100, 101, 144, 145, 146, 147, 148, 149, 150, 151, 152, 153, 154, 155, 156, 157, 158, 159, 160, 161, 162, 163, 164, 165, 166, 167, 168, or 169,
wherein the X in any one of SEQ ID NOs: 76, 77, 78, 79, 80, 81, 82, 83, 84, 85, 86, 87, 88, 89, 90, 91, 92, 93, 94, 95, 96, 97, 98, 99, 100, 101, 144, 145, 146, 147, 148, 149, 150, 151, 152, 153, 154, 155, 156, 157, 158, 159, 160, 161, 162, 163, 164, 165, 166, 167, 168, and 169 is glutamine or pyroglutamate.

84. The isolated polynucleotide of claim 72, wherein:
(a) the antibody comprises a light chain constant region comprising the amino acid sequence of SEQ ID NO: 122 or 123; or
(b) the antibody comprises a light chain comprising the amino acid sequence of SEQ ID NO: 102, 103, 104, 105, 106, 107, 108, 109, 110, 111, 112, 113, 114, or 115.

85. The isolated polynucleotide of claim 83, wherein the heavy chain and light chain comprise the amino acid sequences of SEQ ID NOs: 76 and 102; 79 and 103; 78 and 103; 82 and 103; 84 and 104; 83 and 104; 86 and 103; 85 and 103; 81 and 103; 80 and 103; 87 and 105; 77 and 102; 88 and 102; 77 and 106; 77 and 107; 77 and 108; 77 and 103; 89 and 102; 90 and 102; 91 and 102; 92 and 102; 93 and 102; 77 and 109; 94 and 102; 95 and 102; 96 and 102; 97 and 102; 98 and 102; 99 and 102; 100 and 110; 100 and 111; 100 and 112; 100 and 113; 100 and 114; 100 and 115; 101 and 110; 144 and 102; 147 and 103; 146 and 103; 150 and 103; 152 and 104; 151 and 104; 154 and 103; 153 and 103; 149 and 103; 148 and 103; 155 and 105; 145 and 102; 156 and 102; 145 and 106; 145 and 107; 145 and 108; 145 and 103; 157 and 102; 158 and 102; 159 and 102; 160 and 102; 161 and 102; 145 and 109; 162 and 102; 163 and 102; 164 and 102; 165 and 102; 166 and 102; 167 and 102; 168 and 110; 168 and 111; 168 and 112; 168 and 113; 168 and 114; 168 and 115; or 169 and 110, respectively, wherein the X in any one of SEQ ID NOs: 76-101 or 144-169 is glutamine or pyroglutamate.

86. An isolated polynucleotide encoding an antibody that specifically binds to human CD96, wherein:
(a) the antibody binds to the same epitope of human CD96 as the antibody of claim 72; and/or
(b) the antibody competes for binding to human CD96 with the antibody of claim 72.

87. The isolated polynucleotide of claim 72, wherein:
(a) the antibody is a human antibody; or
(b) the antibody is a multispecific antibody.

88. A vector comprising the polynucleotide of claim 72.

89. A recombinant host cell comprising the polynucleotide of claim 72.

90. A recombinant host cell comprising the vector of claim 88.

91. A pharmaceutical composition comprising:
(a) the polynucleotide of claim 72;
(b) a vector comprising the polynucleotide of claim 72; or
(c) a host cell comprising the vector,
and a pharmaceutically acceptable carrier or excipient.

92. A method of producing an antibody that specifically binds to human CD96, the method comprising culturing the host cell of claim 90 under suitable conditions so that the polynucleotide is expressed and the antibody is produced.

93. A method of increasing an immune response in a subject, the method comprising administering to the subject an effective amount of:
(a) an antibody that specifically binds to human CD96, the antibody comprising a heavy chain variable region (VH) comprising complementarity determining regions (CDRs) CDRH1, CDRH2, and CDRH3, and a light chain variable region (VL) comprising CDRs CDRL1, CDRL2, and CDRL3, wherein:
(i) CDRH1 comprises the amino acid sequence of X₁YX₂X₃X₄ (SEQ ID NO: 135), wherein
X₁ is Q or S;
X₂ is A or S;
X₃ is M or I; and
X₄ is H or S;
(ii) CDRH2 comprises the amino acid sequence of
X₁IX₂X₃X₄X₅X₆X₇X₈X₉YX₁₀QKFQG (SEQ ID NO: 137), wherein
X₁ is W or G;
X₂ is N or I;
X₃ is A, E, V, or P;
X₄ is V, G, W, or I;
X₅ is S, Y, T, N, or F;
X₆ is G or W;
X₇ is D, Y, N, or T;
X₈ is T or A;
X₉ is K or N; and
X₁₀ is S or A;
(iii) CDRH3 comprises the amino acid sequence of NWGX₁SYGX₂DV (SEQ ID NO: 180), SEQ ID NO: 19, or SEQ ID NO: 20, wherein
X₁ is M or L; and
X₂ is M or L;
(iv) CDRL1 comprises the amino acid sequence of RASQSIX₁X₂YLN (SEQ ID NO: 139) or SEQ ID NO: 26, wherein
X₁ is S, T, or L; and
X₂ is S, P, or W;
(v) CDRL2 comprises the amino acid sequence of X₁X₂SSLQS (SEQ ID NO: 141) or SEQ ID NO: 32, wherein
X₁ is S or A; and
X₂ is A, S, or E; and/or
vi) CDRL3 comprises the amino acid sequence of QQX₁YSTPALX₂ (SEQ ID NO: 143) or SEQ ID NO: 35, wherein
X₁ is S or A; and
X₂ is T or S;
(b) the polynucleotide of claim 72;
(c) a vector comprising the polynucleotide of claim 72;
(d) a host cell comprising
(i) the polynucleotide of claim 72;
(ii) a vector comprising the polynucleotide of claim 72; or
(e) a pharmaceutical composition comprising the antibody of (a), the polynucleotide of (b), the vector of (c), or the host cell of (d), and a pharmaceutically acceptable carrier or excipient, optionally wherein the antibody, polynucleotide, vector, host cell, or pharmaceutical composition is administered, systemically, intravenously, subcutaneously, or intratumorally, or is delivered to a tumor draining lymph node.
